(19) United States
(12) Patent Application Publication
(10) Pub. No.: US 2026/0287586 A1
(43) Pub. Date: Sep. 24, 2026

(54) METHODS OF TREATING TKI-RESISTANT AND SDH-DEFICIENT GIST

(71) Applicants: GUANGZHOU HEALTHQUEST PHARMA CO., LTD., Guangzhou, Guangdong (CN); ASCENTAGE PHARMA (SUZHOU) CO., LTD., Suzhou, Jiangsu (CN); ASCENTAGE PHARMA GROUP CORP LIMITED, Hong Kong (CN)

(72) Inventor: Yifan ZHAI

(21) Appl. No.: 19/477,116

(22) PCT Filed: Apr. 26, 2024
(86) PCT No.: PCT/CN2024/090159

§ 371 Date: Oct. 21, 2025

(30) Foreign Application Priority Data
Apr. 26, 2023 (WO) PCT/CN2023/090999
Mar. 29, 2024 (WO) PCT/CN2024/084837

(51) Int. Cl. G01N 33/573; A61K 31/496; A61K 45/06; A61P 35/00; G01N 33/575

(52) U.S. Cl. CPC G01N 33/573; A61K 31/496; A61K 45/06; A61P 35/00; G01N 33/5758; G01N 2333/90206; G01N 2800/52

(57) ABSTRACT

Provided herein is a method of treating a disease, disorder or condition associated with succinate dehydrogenase- (SDH-) deficiency with a multi-targeted tyrosine kinase inhibitor (TKI). Also provided herein is a method of treating a disease with the combination of multi-targeted TKI with Bcl-2 inhibitor.

### FIELD OF THE INVENTION

The present invention relates to a method of treating a disease, disorder or condition associated with succinate dehydrogenase- (SDH-) deficiency with a multi-targeted tyrosine kinase inhibitor (TKI). The present invention also relates to a method of treating a disease with the combination of a multi-targeted TKI with a BCL-2 inhibitor.

### BACKGROUND OF THE INVENTION

Gastrointestinal stromal tumors (GISTs) are the most common mesenchymal malignancies of the GI tract (Unc, M. et al. Cancers (Basel) 15, 1498 (2023)). The pathogenesis of about 85% to 90% of them is driven by oncogenic mutations in genes encoding KIT or platelet-derived growth factor receptor alpha (PDGFRA) tyrosine kinases. Others without KIT or PDGFRA mutations are wild-type (WT) GISTs, of which succinate dehydrogenase (SDH)-deficient GISTs are the most common, accounting for 5% to 7.5% of all GISTs (Unc, M. et al. Cancers (Basel) 15, 1498 (2023)). SDH-deficient GISTs typically occur because of germline and/or somatic loss-of-function mutations in any of four SDH subunits (A, B, C, or D) (Pasini, B., et al. Eur. J. Hum. Genet. 16, 79-88 (2008)), or tumors having methylation of the SDHC promoter, also leading to silencing of SDH expression. Clinically, SDH-deficient GISTs, compared with KIT/PDGFRA mutant GISTs, tend to be multifocal and display an indolent clinical course, even when advanced or metastatic (Nannini, M., et al. Ther. Adv. Med. Oncol. 13, 17588359211023278 (2021)).

Surgery is the only potentially curative treatment option for patients with SDH-deficient GISTs (Unc, M. et al. Cancers (Basel) 15, 1498 (2023)). Other therapeutic alternatives include systemic or targeted therapy, such as tyrosine kinase inhibitors (TKIs). The introduction and use of TKIs improved the treatment landscape for GISTs, especially imatinib as first-line treatment in patients with KIT or PDGFRA mutations. Other TKIs such as sunitinib and regorafenib are approved as second- and third-line treatment options in patients who develop imatinib resistance. However, the role of TKIs in patients with SDH-deficient GIST remains controversial, with limited published results. Imatinib has no proven efficacy in this type (Unc, M. et al. Cancers (Basel) 15, 1498 (2023)). The other approved TKIs have limited efficacy, and studies of these agents in metastatic SDH-deficient GISTs have shown a median progression-free survival (PFS) of less than 10 months (Nannini, M., et al. Ther. Adv. Med. Oncol. 13, 17588359211023278 (2021)). These factors highlight an unmet clinical need for patients with SDH-deficient GISTs, especially those are not sensitive to prior PKI therapy.

### SUMMARY OF THE INVENTION

Throughout the present disclosure, the articles “a”, “an”, and “the” are used herein to refer to one or to more than one (i.e., to at least one) of the grammatical object of the article. By way of example, “a method” means one method or more than one method.

Throughout the present disclosure, all the references cited herein are incorporated in their entirety.

It has now been found by the inventors of the present application that the administration of a multi-targeted TKI (e.g., olverembatinib) is particularly effective in treating a disease, disorder or condition associated with SDH-deficiency (e.g., GIST), especially for those subjects not sensitive to at least one prior TKI treatment. It has also been found by the inventors of the present application that the administration of a multi-targeted TKI (e.g., olverembatinib) in combination with a BCL-2 inhibitor (e.g., lisaftoclax) is particularly effective in treating a cancer (e.g., GIST).

In one aspect, the present disclosure provides a method of treating a disease, disorder or condition associated with succinate dehydrogenase- (SDH-) deficiency in a subject, comprising administering a therapeutically effective amount of a multi-targeted tyrosine kinase inhibitor (TKI) to the subject, thereby treating the disease, disorder or condition associated with SDH-deficiency in the subject.

In another aspect, the present disclosure provides a method of identifying a subject having a disease, disorder or condition associated with succinate dehydrogenase- (SDH-) deficiency and selecting a treatment for the identified subject, comprising:
a) determining if a biological sample from the subject is SDH-deficient;
b) identifying the subject as having the disease, disorder or condition associated with SDH-deficiency, based on the SDH-deficiency determined in step a), and
c) recommending the subject to receive treatment of a multi-targeted TKI.

In some embodiments, the multi-targeted TKI comprises olverembatinib.

In some embodiments, the disease, disorder or condition associated with SDH-deficiency is cancer (e.g. SDH-deficient cancer).

In some embodiments, the cancer is selected from the group consisting of: gastrointestinal cancer (e.g., gastrointestinal stromal tumor (GIST)), cholangiocarcinoma, lung cancer, melanoma, breast cancer, colon cancer, ovarian cancer, prostate cancer, liver cancer (e.g., hepatocellular carcinoma), bladder cancer, pancreatic cancer, renal cancer, esophageal cancer, head and neck cancer, thyroid cancer, cutaneous squamous cell carcinoma, glioblastoma. neuroblastoma, urinary bladder cancer, hysterocarcinoma, melanoma, osteosarcoma, lymphoma (e.g., mantel cell lymphoma, diffuse large B cell lymphoma), leukemia (e.g., T-cell prolymphocytic leukemia, chronic lymphocytic leukemia, acute myeloid leukemia, acute lymphocytic leukemia (ALL)), multiple myeloma, uterine cancer, colorectal cancer, lung adenocarcinoma, uterine carcinosarcoma, lung squamous cell carcinoma, cervical cancer, esophagus cancer, sarcoma, chromophobe, renal cell carcinoma, clear cell RCC, papillary RCC, uveal melanoma, testicular germ cell tumor, low grade glioma (LGG), mesothelioma, pheochromocytoma, paraganglioma, thymoma, and adenoid cystic carcinoma (ACC).

In some embodiments, the cancer is SDH-deficient GIST.

In some embodiments, the SDH-deficient cancer is selected from the group consisting of SDH-deficient ALL, renal cell carcinoma, colon cancer, pheochromocytoma, thyroid neoplasia (e.g., papillary thyroid neoplasia, multifocal papillary thyroid neoplasia, differentiated thyroid cancer, papillary (follicular variant) thyroid neoplasia, follicular thyroid neoplasia), pancreatic neuroendocrine tumor/intrapancreatic paraganglioma, breast cancer (e.g., breast carcinoma (triple negative)), neuroblastoma, follicular lymphoma, b-cell lymphoma, Hodgkin's lymphoma, composite paraganglioma/neuroblastoma, rectal adenocarcinoma, Burkitt's lymphoma, thyroid paraganglioma, adrenocortical carcinoma, adenomatoid tumor of the adrenal gland, neuroblastoma, melanoma, synovial sarcoma, lung squamous cell carcinoma, embryonal rhabdomyosarcoma, lung adenocarcinoma, prostate adenocarcinoma, endometrial carcinoma, poorly differentiated carcinoma of the stomach, urothelial bladder cancer, colon adenocarcinoma, adenocarcinoma of the sigmoid colon, testicular seminoma, and pituitary adenoma.

In some embodiments, the subject has been determined as having SDH-deficiency.

In some embodiments, the SDH-deficiency is determined in a biological sample from the subject.

In some embodiments, the biological sample is tissue (e.g., tumor tissue), fluid (e.g., whole blood, plasma, PBMC) or any biological samples that can be used for detection of germline/somatic mutation of SDH genes.

In some embodiments, the SDH-deficiency is determined by immunohistochemistry, western blot, amplification-based assay, hybridization based assay, or sequencing.

In some embodiments, the SDH-deficiency is indicated by presence of one or more loss of function mutations in SDHA, SDHB, SDHC, and SDHD, or any combination thereof, or indicated by SDHC promoter methylation, that causes reduction in SDH enzyme activity, destabilization of SDH protein complex, or reduction in SDH expression.

In some embodiments, the one or more loss of function mutations comprise a mutation selected from the group consisting of missense substitution, deletion, insertion, translocation, and premature stop codon, or any combination thereof.

In some embodiments, the one or more loss of function mutations in SDHB comprises one or more mutations selected from the group consisting of p.A15T, p.W218Ter, and SDHB KO exon 2+3 (Heterozygote).

In some embodiments, the one or more loss of function mutations in SDHB comprises one or more mutations selected from the group consisting of c.17_42dup, c.71_72insA, c.136C>T, c.137G>A, c.274T>A, c.301delT, c.380T>G, c.423+1G>A, c.423+20T>A, c.566G>T, c.600G>T, c.643-1G>C, c.725G>A, and c.778_779insG, or any combination thereof.

In some embodiments, the one or more loss of function mutations in SDHA comprises one or more mutations selected from the group consisting of p.G184R and p.P279S.

In some embodiments, the one or more loss of function mutations in SDHA comprises: c.91C>T, c.113A>T, c.160C>T, c.511C>T, c.533C>T, c.457-3_457-1delCAG, c.562C>T, c.628C>T, c.666del, c.688delG, c.767C>T, c.800C>T, c.818C>T, c.923C>T, c.985C>T, c.1043-1055del, c.1046_1047delTG, c.1054C>T, c.1151C>G, c.1215_1216insCC, c.1255G>A, c.1334C>T, c.1352G>A, c.1360G>A, c.1361C>A, c. 1367C>T, c.1468delG, c.1513delA, c.1532T>C, c.1534C>T, c.1753C>T, c.1765C>T, c.1766G>A, c.1690G>A, c.1794G>C, c.1795-1G>T, c.1798C>T, and c. 1799G>A, or any combination thereof.

In some embodiments, the one or more loss of function mutations in SDHC comprises one or more mutations selected from the group consisting of: c.1A>G, c.6delT, c.65G>A, c.160C>A, c.221A>G, c.224G>A, c.238C>T, c.301delT, c.380A>G, c.397C>T, and c.405+1G>A, or any combination thereof.

In some embodiments, the one or more loss of function mutations in SDHD comprises one or more mutations selected from the group consisting of: c.34G>A, c.352delG, c.416T>C, and c.455G>A, or any combination thereof.

In some embodiments, the subject (e.g., the biological sample of the subject) has further been determined as having wild-type KIT, wild-type PDGFR, or combination thereof.

In some embodiments, the subject (e.g., the biological sample of the subject) has been determined as comprising one or more mutations in NF1/BRAF/RAS pathway genes, PIK3CA, or fusions affecting NTRK3, or any combination thereof.

In some embodiments, the subject (e.g. the biological sample of the subject) has been determined as overexpressing VEGFR or FGF ligand.

In some embodiments, the subject has been determined as not sensitive to at least one existing TKI.

In some embodiments, the subject has not obtained complete response (CR), partial response (PR), or stable disease (SD) within 6 months of the treatment of the at least one existing TKI; or obtaining initial response or stable disease following ≥6 months of the treatment of the at least one existing TKI and subsequent progression.

In some embodiments, the subject has been determined as not sensitive to at least one existing TKIs selected from the group consisting of imatinib, ripretinib, avapritinib, crenolanib, masitinib, ponatinib, dasatinib, nilotinib, sunitinib, regorafenib, sorafenib, anlotinib, sulfatinib, pazopanib, rogaratinib, infigratinib, erdafitinib, pemigatinib, fisogatinib, and famitinib, or any combinations thereof.

In some embodiments, the subject has been determined as not sensitive to at least one existing TKIs selected from the group consisting of ripretinib, dasatinib, sunitinib, regorafenib, anlotinib, and famitinib, or any combinations thereof.

In some embodiments, the multi-targeted TKI is capable of inhibiting at least two tyrosine kinases selected from the group consisting of: KIT, PDGFR, SRC, VEGFR (e.g., any one of VEGFR1, VEGFR2, and VEGFR3, or any combination thereof), and FGFR (e.g., any one of FGFR1, FGFR2, FGFR3, and FGFR4, or any combination thereof), or any combination thereof.

In some embodiments, the multi-targeted TKI is capable of inhibiting at least two tyrosine kinases selected from the group consisting of: KIT, PDGFR, SRC, VEGFR2, and FGFR1, or any combination thereof.

In some embodiments, the multi-targeted TKI is capable of inhibiting proliferation of SDH-deficient tumor cells.

In some embodiments, the multi-targeted TKI has an IC₅₀ of no more than 10 μM (e.g., no more than 5 μM, 1 μM, 500 nM, 400 nM, 300 nM, 200 nM, 150 nM, 100 nM, 50 nM, 20 nM, or 10 nM) in inhibiting proliferation of SDH-deficient tumor cells, as determined by CCK8 assay or CTG assay.

In some embodiments, the SDH-deficient tumor cell is Jurkat clone E6-1, OS-RC-2, RKO, and PC12 #5F7, or any combination thereof.

In some embodiments, the multi-targeted TKI comprises a heterocyclic alkyne benzene compound having the following formula (I):

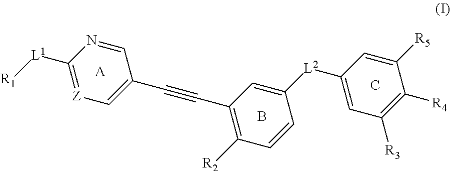

wherein Z is independently selected as CH or N; L¹ is independently selected from NH, —N═, CH; L² is independently selected as —CONH— or —NHCO—;
R₁ is independently selected from:
   1) H;
   2) C₁-C₆ alkyl;
   3) C₃-C₆ cycloalkyl;
   4) C₁-C₅ alkyl substituted by 1 or 2 hydroxy;
   5) phenyl;
   6) Groups which can fuse A ring through L¹ in Z atom site to form fused penta-heterocycles containing 1-3 N atoms and L¹ like

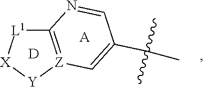

wherein, X, Y, Z are independently selected as N, CH; ring D is an aromatic heterocycle containing 1-3 N atoms;
R₂ is independently selected from
   1) H;
   2) Halogen;
   3) C₁-C₅ alkyl;
   4) C₃-C₆ cycloalkyl;
   5) C₁-C₅alkyl containing F;
R₃ is independently selected from
   1) H;
   2) Halogen;
   3) C₁-C₄ alkyl;
   4) C₃-C₆ cycloalkyl;
   5) C₁-C₄ alkyl containing F;
when R₅ is H, R₄ is independently selected from
   1) H;
   2) (CH₂)ₙNR₆R₇;
   3) (CH₂)ₙ-Het¹;
or, when R₄ is H, R₅ is independently selected from
   1) H;
   2) Het²;
   n is independently selected from 0 or 1; Het¹ is defined as nonaromatic heterocycle containing 1-3 N atoms; Het² is defined as aromatic five-member heterocycle containing hetero atoms like N, O, and/or S; alkyl, cycloalkyl or NR₆R₇ may be incorporated into any C or N position in the nonaromatic heterocycle and aromatic heterocycle which can be substituted;
R₆ or R₇ is independently selected from:
   1) H;
   2) C₁-C₃ alkyl;
   3) C₁-C₃ alkyl containing F;
   4) C₃-C₆ cycloalkyl;
   or R₆ and R₇ can further form penta-, hexa-, hepta- or octatomic ring structure through C, O, N, S atoms;
or a pharmaceutically acceptable salt or a stereoisomer or a prodrug thereof.

In some embodiments, the multi-targeted TKI is a compound having the following formula (II):

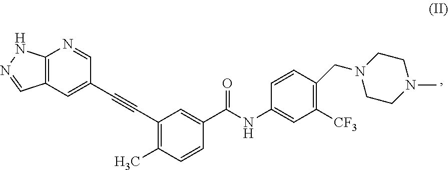 [SMILES: Cc1ccc(C(=O)Nc2ccc(CN3CCN(C)CC3)c(C(F)(F)F)c2)cc1C#Cc1cnc2[nH]ncc2c1]

or a pharmaceutically acceptable salt or a stereoisomer or a prodrug thereof.

In some embodiments, the multi-targeted TKI is a compound selected from the group consisting of: olverembatinib, ripretinib, avapritinib, crenolanib, masitinib, ponatinib, dasatinib, nilotinib, sunitinib, regorafenib, sorafenib, anlotinib, sulfatinib, infigratinib, erdafitinib, pemigatinib, rogaratinib, and linsitinib, or a pharmaceutically acceptable salt or a stereoisomer or a prodrug of any of the foregoing.

In some embodiments, the multi-targeted TKI is administered at a dose from about 10 mg to 200 mg, 10 mg to 150 mg, 10 mg to 100 mg, 10 mg to 50 mg, or 20 mg to 50 mg.

In some embodiments, the multi-targeted TKI is administered at a dose of about 10 mg, 20 mg, 30 mg, 40 mg, or 50 mg.

In some embodiments, the multi-targeted TKI is administered once a day, once every other day, once a week, once every other week, once a month, or once every other month.

In some embodiments, the treatment comprises one or more 28-days treatment cycles.

In some embodiments, the treatment comprises at least 1, 2, 3, 6, 14, 16 or 36 treatment cycles.

In some embodiments, the multi-targeted TKI is administered by oral route or parental route (e.g., intravenous, intramuscular, subcutaneous, intramedullary, intrathecal, intraventricular, intraperitoneal, intranasal, or intraocular injection).

In some embodiments, the multi-targeted TKI is orally administered once every other day in 28-days treatment cycles.

In some embodiments, the method further comprises further administering a therapeutically effective amount of one or more additional therapies.

In some embodiments, the multi-targeted TKI is administered in combination with one or more additional therapies that treat the disease, disorder or condition.

In some embodiments, the one or more additional therapies comprise a radiotherapy, a chemotherapy, a targeted cancer therapy, a therapy with a modulator of an immune checkpoint molecule, or a therapy with a BCL-2 inhibitor.

In some embodiments, following the treatment of the compound having formula (II), the subject experiences partial response or stable disease.

In some embodiments, following cycles of 28-days treatment cycle of the compound having formula (II), the objective response rate (complete response and partial response) is about or above 20%, and/or the clinical benefit rate (complete response, partial response and stable disease) is about or above 90%.

In another aspect, the present disclosure provides a method of treating cancer in a subject, comprising administering to the subject,
a) a therapeutically effective amount of a multi-targeted tyrosine kinase inhibitor (TKI); and
b) a therapeutically effective amount of a Bcl-2 inhibitor.

In some embodiments, the cancer is selected from the group consisting of: gastrointestinal cancer (e.g., gastrointestinal stromal tumor (GIST)), cholangiocarcinoma, lung cancer, melanoma, breast cancer, colon cancer, ovarian cancer, prostate cancer, liver cancer (e.g. hepatocellular carcinoma), bladder cancer, pancreatic cancer, renal cancer, esophageal cancer, head and neck cancer, thyroid cancer, cutaneous squamous cell carcinoma, glioblastoma. neuroblastoma, urinary bladder cancer, hysterocarcinoma, melanoma, osteosarcoma, lymphoma (e.g., mantel cell lymphoma, diffuse large B cell lymphoma), leukemia (e.g., T-cell prolymphocytic leukemia, chronic lymphocytic leukemia, acute myeloid leukemia, acute lymphocytic leukemia (ALL)), multiple myeloma, uterine cancer, colorectal cancer, lung adenocarcinoma, uterine carcinosarcoma, lung squamous cell carcinoma, cervical cancer, esophagus cancer, sarcoma, chromophobe, renal cell carcinoma, clear cell RCC, papillary RCC, uveal melanoma, testicular germ cell tumor, low grade glioma (LGG), mesothelioma, pheochromocytoma, paraganglioma, thymoma, and adenoid cystic carcinoma (ACC).

In some embodiments, the cancer is GIST.

In some embodiments, the subject has been determined as comprising one or more mutations in KIT, PDGFR (e.g., PDGFRA and PDGFRB), SDH (e.g., SDHA, SDHB, SDHC and SDHD), NF1, BRAF, EGFR, HRAS, NRAS, KRAS, ZNF217, MEN1, PIK3CA, ARID1A, ARID1B, FGFR1, FGFR2, FGFR3, FGFR4, SDHAF2, FGFR1-HOOK3, FGFR1-TACC1, NTRK1 (e.g., NTRK1 fusions), NTRK2 (e.g., NTRK2 fusions), NTRK3 (e.g., NTRK3 fusions), KIT-PDGFRA, and PRKAR1B-BRAF, or any combination thereof.

In some embodiments, the subject has been determined as not sensitive to at least one existing TKI.

In some embodiments, the subject has not obtained complete response (CR), partial response (PR), and stable disease (SD) within 6 months of the treatment of the at least one existing TKI; or obtaining initial response or stable disease following ≥6 months of the treatment of the at least one existing TKI and subsequent progression.

In some embodiments, the subject has been determined as not sensitive to at least one existing TKIs selected from the group consisting of imatinib, ripretinib, avapritinib, crenolanib, masitinib, ponatinib, dasatinib, nilotinib, sunitinib, regorafenib, sorafenib, anlotinib, sulfatinib, pazopanib, rogaratinib, infigratinib, erdafitinib, pemigatinib, fisogatinib, and famitinib, or any combination thereof.

In some embodiments, the subject has been determined as not sensitive to at least one existing TKIs selected from the group consisting of ripretinib, dasatinib, sunitinib, regorafenib, anlotinib, and famitinib, or any combinations thereof.

In some embodiments, the multi-targeted TKI is capable of inhibiting at least two tyrosine kinases selected from the group consisting of: KIT, PDGFR, SRC, VEGFR (e.g., any one of VEGFR1, VEGFR2, and VEGFR3, or any combination thereof), and FGFR (e.g., any one of FGFR1, FGFR2, FGFR3, and FGFR4, or any combination thereof), or any combinations thereof.

In some embodiments, the multi-targeted TKI is capable of inhibiting at least two tyrosine kinases selected from the group consisting of: KIT, PDGFR, SRC, VEGFR2, and FGFR1, or any combination thereof.

In some embodiments, the multi-targeted TKI comprises a heterocyclic alkyne benzene compound having the following formula (I):

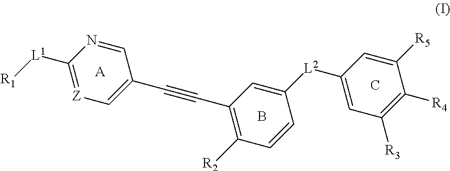

wherein Z is independently selected as CH or N; L¹ is independently selected from NH, —N═, CH; L² is independently selected as —CONH— or —NHCO—;
R₁ is independently selected from:
   1) H;
   2) C₁-C₆ alkyl;
   3) C₃-C₆ cycloalkyl;
   4) C₁-C₅ alkyl substituted by 1 or 2 hydroxy;
   5) phenyl;
   6) Groups which can fuse A ring through L¹ in Z atom site to form fused penta-heterocycles containing 1-3 N atoms and L¹ like

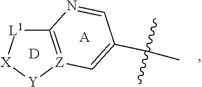

wherein, X, Y, Z are independently selected as N, CH; ring D is an aromatic heterocycle containing 1-3 N atoms;
R₂ is independently selected from
   1) H;
   2) Halogen;
   3) C₁-C₅ alkyl;
   4) C₃-C₆ cycloalkyl;
   5) C₁-C₅ alkyl containing F;
R₃ is independently selected from
   1) H;
   2) Halogen;
   3) C₁-C₄ alkyl;
   4) C₃-C₆ cycloalkyl;
   5) C₁-C₄ alkyl containing F;
when R₅ is H, R₄ is independently selected from
   1) H;
   2) (CH₂)ₙNR₆R₇;
   3) (CH₂)ₙ-Het¹;
or, when R₄ is H, R₅ is independently selected from
   1) H;
   2) Het²;
   n is independently selected from 0 or 1; Het¹ is defined as nonaromatic heterocycle containing 1-3 N atoms; Het² is defined as aromatic five-member heterocycle containing hetero atoms like N, O, and/or S; alkyl, cycloalkyl or NR₆R₇ may be incorporated into any C or N position in the nonaromatic heterocycle and aromatic heterocycle which can be substituted;
R₆ or R₇ is independently selected from:
   1) H;
   2) C₁-C₃ alkyl;
   3) C₁-C₃ alkyl containing F;
   4) C₃-C₆ cycloalkyl;
   or R₆ and R₇ can further form penta-, hexa-, hepta- or octatomic ring structure through C, O, N, S atoms;
or a pharmaceutically acceptable salt or a stereoisomer or a prodrug thereof.

In some embodiments, the multi-targeted TKI is a compound having the following formula (II):

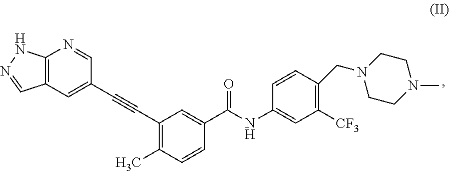 [SMILES: Cc1ccc(C(=O)Nc2ccc(CN3CCN(C)CC3)c(C(F)(F)F)c2)cc1C#Cc1cnc2[nH]ncc2c1]

or a pharmaceutically acceptable salt or a stereoisomer or a prodrug thereof.

In some embodiments, the multi-targeted TKI is a compound selected from the group consisting of: olverembatinib, ripretinib, avapritinib, crenolanib, masitinib, ponatinib, dasatinib, nilotinib, sunitinib, regorafenib, sorafenib, anlotinib, sulfatinib, infigratinib, erdafitinib, pemigatinib, rogaratinib, and linsitinib, or a pharmaceutically acceptable salt or a stereoisomer or a prodrug of any of the foregoing.

In some embodiments, the multi-targeted TKI is administered at a dose of about 1 mg/kg, 5 mg/kg, 10 mg/kg, 15 mg/kg, 20 mg/kg, 25 mg/kg, 50 mg/kg, 75 mg/kg, 100 mg/kg, 150 mg/kg, or 200 mg/kg.

In some embodiments, the multi-targeted TKI is administered once a day, once every other day, once a week, once every other week, once a month, or once every other month.

In some embodiments, the multi-targeted TKI is administered for at least 1 week, 2 weeks, 3 weeks, 4 weeks, 2 months, 3 months, or 4 months.

In some embodiments, the multi-targeted TKI is administered by oral route or parental route (e.g., intravenous, intramuscular, subcutaneous, intramedullary, intrathecal, intraventricular, intraperitoneal, intranasal, or intraocular injection).

In some embodiments, the multi-targeted TKI is administered once every other day for 3 weeks at a dose of about 15 mg/kg.

In some embodiments, the Bcl-2 inhibitor is a compound having the following formula (III)

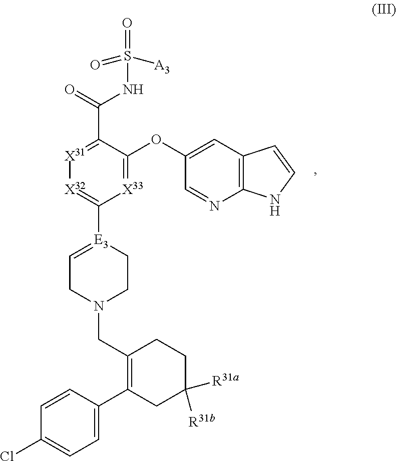

or a pharmaceutically acceptable salt or a stereoisomer or a prodrug thereof, wherein:

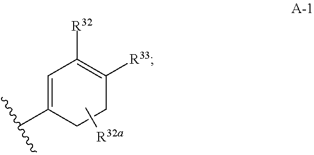

A₃ is selected from
E₃ is a nitrogen atom and [symbol] is a single bond;
X³¹, X³², and X³³ are each independently selected from the group consisting of —CR³⁸═ and —N═;
R^{31a} and R^{31b} taken together with the carbon atom to which they are attached form a 3-, 4-, or 5-membered cycloalkyl;
R³² is selected from the group consisting of —NO₂, —SO₂CH₃, and —SO₂CF₃;
R^{32a} is selected from the group consisting of hydrogen and halogen;
R³³ is selected from —N(R^{34a})(R^{34b});
R^{34a} is selected from the group consisting of optionally substituted C₁₋₆ alkyl, optionally substituted C₃₋₆ cycloalkyl, heterocyclo, heteroalkyl, (cycloalkyl)alkyl, and (heterocyclo)alkyl;
R^{34b} is selected from the group consisting of hydrogen and C₁₋₄ alkyl;
R³⁸ is selected from the group consisting of hydrogen and halogen.

In some embodiments, the Bcl-2 inhibitor is selected from the group consisting of:

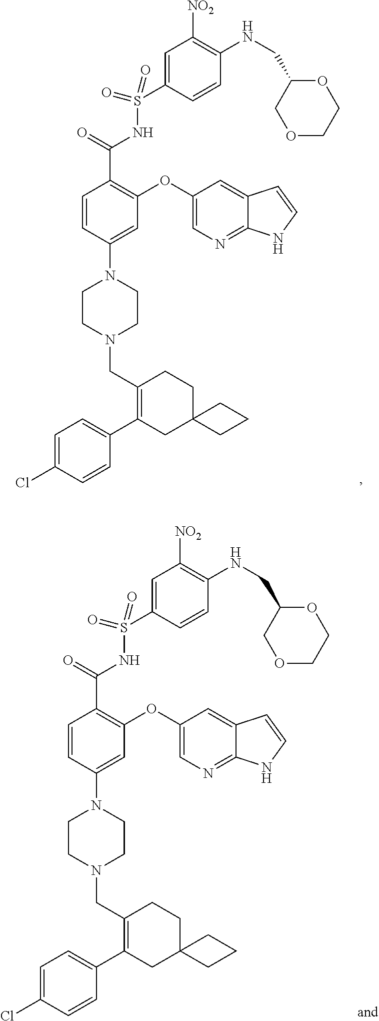 [SMILES: O=C(NS(=O)(=O)c1ccc(NC[C@H]2COCCO2)c([N+](=O)[O-])c1)c1ccc(N2CCN(CC3=C(c4ccc(Cl)cc4)CC4(CCC4)CC3)CC2)cc1Oc1cnc2[nH]ccc2c1 ; O=C(NS(=O)(=O)c1ccc(NC[C@@H]2COCCO2)c([N+](=O)[O-])c1)c1ccc(N2CCN(CC3=C(c4ccc(Cl)cc4)CC4(CCC4)CC3)CC2)cc1Oc1cnc2[nH]ccc2c1]

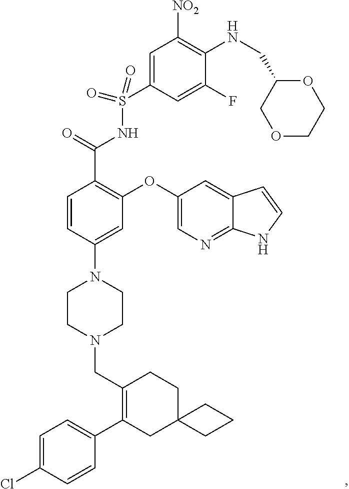 [SMILES: O=C(NS(=O)(=O)c1cc(F)c(NC[C@H]2COCCO2)c([N+](=O)[O-])c1)c1ccc(N2CCN(CC3=C(c4ccc(Cl)cc4)CC4(CCC4)CC3)CC2)cc1Oc1cnc2[nH]ccc2c1]

or a pharmaceutically acceptable salt or a stereoisomer or a prodrug thereof.

In some embodiments, the Bcl-2 inhibitor is a compound having the following formula (IV)

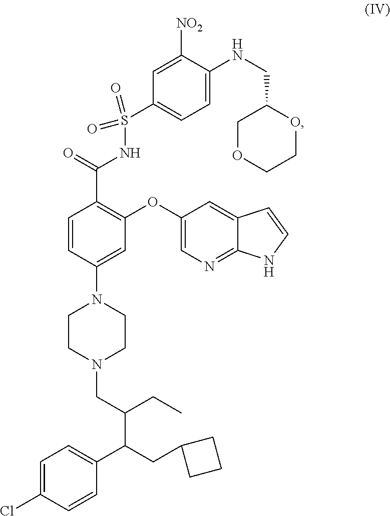 [SMILES: CCC(CN1CCN(c2ccc(C(=O)NS(=O)(=O)c3ccc(NC[C@H]4COCCO4)c([N+](=O)[O-])c3)c(Oc3cnc4[nH]ccc4c3)c2)CC1)C(CC1CCC1)c1ccc(Cl)cc1]

or a pharmaceutically acceptable salt or a stereoisomer or a prodrug thereof.

In some embodiments, the Bcl-2 inhibitor is administered at a dose of about 1 mg/kg, 5 mg/kg, 10 mg/kg, 15 mg/kg, 20 mg/kg, 25 mg/kg, 50 mg/kg, 75 mg/kg, 100 mg/kg, 150 mg/kg, or 200 mg/kg.

In some embodiments, the Bcl-2 inhibitor is administered twice a day, once a day, once every other day, once a week, once every other week, once a month, or once every other month.

In some embodiments, the Bcl-2 inhibitor is administered for at least 1 week, 2 weeks, 3 weeks, 4 weeks, 2 months, 3 months or 4 months.

In some embodiments, the Bcl-2 inhibitor is administered by oral route or parental route (e.g., intravenous, intramuscular, subcutaneous, intramedullary, intrathecal, intraventricular, intraperitoneal, intranasal, or intraocular injection).

In some embodiments, the Bcl-2 inhibitor is administered once a day for 3 weeks at a dose of about 50 mg/kg.

In some embodiments, the multi-targeted TKI and the Bcl-2 inhibitor are administered concurrently.

In some embodiments, the multi-targeted TKI and the Bcl-2 inhibitor are administered sequentially.

In some embodiments, the multi-targeted TKI is administered once every other day for 3 weeks at a dose of about 15 mg/kg and the Bel-2 inhibitor is administered once a day for 3 weeks at a dose of about 50 mg/kg.

In some embodiments, the multi-targeted TKI in combination with the Bcl-2 inhibitor treat the cancer in the subject in an additive manner or a synergistic manner.

In some embodiments, following the treatment of the compound having formula (II) in combination with the compound having formula (IV), the subject has tumor growth inhibition (TGI) rates of at least 75%.

In some embodiments, the multi-targeted TKI in combination with the Bel-2 inhibitor are capable of inhibiting proliferation of imatinib-resistant GIST cells.

In some embodiments, the multi-targeted TKI in combination with the Bcl-2 inhibitor are capable of inhibiting proliferation of imatinib-resistant GIST cells in an additive manner or a synergistic manner.

In some embodiments, the imatinib-resistant GIST cell is GIST430, GIST48, and GIST48B, or any combination thereof.

In another aspect, the present disclosure provides a method of treating a disease, disorder or condition associated with upregulated hypoxia inducible factors (HIF-1α, HIF-2α), vascular endothelial growth factor-A (VEGFA), fibroblast growth factor receptor-1 (FGFR1), phosphorylated FGFR1, insulin-like growth factor-1 receptor (IGF-1R), phosphorylated IGF-1R, STAT3, phosphorylated STAT3 and/or phosphorylated Src in a subject, comprising administering a therapeutically effective amount of a multi-targeted tyrosine kinase inhibitor (TKI) to the subject, thereby treating the disease, disorder or condition associated with lipid metabolism in the subject.

In some embodiments, the disease, disorder or condition can be cancer.

In some embodiments, the cancer is selected from the group consisting of: gastrointestinal cancer (e.g., gastrointestinal stromal tumor (GIST)), cholangiocarcinoma, lung cancer, melanoma, breast cancer, colon cancer, ovarian cancer, prostate cancer, liver cancer (e.g. hepatocellular carcinoma), bladder cancer, pancreatic cancer, renal cancer, esophageal cancer, head and neck cancer, thyroid cancer, cutaneous squamous cell carcinoma, glioblastoma. neuroblastoma, urinary bladder cancer, hysterocarcinoma, melanoma, osteosarcoma, lymphoma (e.g., mantel cell lymphoma, diffuse large B cell lymphoma), leukemia (e.g., T-cell prolymphocytic leukemia, chronic lymphocytic leukemia, acute myeloid leukemia, acute lymphocytic leukemia (ALL)), multiple myeloma, uterine cancer, colorectal cancer, lung adenocarcinoma, uterine carcinosarcoma, lung squamous cell carcinoma, cervical cancer, esophagus cancer, sarcoma, chromophobe, renal cell carcinoma, clear cell RCC, papillary RCC, uveal melanoma, testicular germ cell tumor, low grade glioma (LGG), mesothelioma, pheochromocytoma, paraganglioma, thymoma, and adenoid cystic carcinoma (ACC).

### BRIEF DESCRIPTION OF THE DRAWINGS

FIGS. 1A-1B show antitumor efficacy of olverembatinib in patients with SDH-deficient GISTs. FIG. 1A shows duration of treatment and best response (RECIST v1.1). Each row represents one patient's duration of treatment with her/his best response. The two left columns show the treatment history and gene mutation of each patient. (Im, imatinib; Sun, sunitinib; Das, dasatinib; Reg, regorafenib; Rep, ripretinib; Anlo, anlotinib; Fam, famitinib; TMZ, temozolomide. PD, progressive disease; PR, partial response; SD, stable disease). FIG. 1B shows Kaplan-Meier estimates of progression-free survival (PFS) by RECIST v1.1. Median PFS was 25.7 months (range 12.9-not reached).

FIG. 2A-2C show potency of olverembatinib in SDH-deficient or knock-down (KD) tumor cells and molecular pathways regulated by olverembatinib. FIG. 2A shows antiproliferation activity of TKIs in SDH-deficient primary GIST cells. Cells were extracted from SDH-deficient patient tumor tissues, seeded into 96-well plates, and incubated for 24 hours in GIST culture medium. Cells were exposed to olverembatinib and other TKIs for 72 hours, then the CellTiter 96® Aqueous Non-Radioactive Cell Proliferation Assay was used to detect cell viability. FIG. 2B shows: Left panel, Cell death assay in rat pheochromocytoma cells PC12 or PC12 #5F7 (SDHB KD). Cells were seeded into 24-well plates, incubated overnight in complete culture medium, exposed to olverembatinib for 24 hours, and then collected for propidium iodide (PI) Staining. Right panel, Colony formation assay in renal carcinoma cells 7860 (SDHB-wild-type) and OS-RC-2 (SDHB-deficient). Cells were seeded into 6-well plates and incubated overnight in complete culture medium. 7860 cells were exposed to olverembatinib for 7 days (and OS-RC-2 cells for 10 days), then stained with crystal violet. FIG. 2C shows olverembatinib regulates multiple oncogenic signaling proteins in SDHB KD PC12 #5F7 cells. After cells were treated with the indicated concentrations of olverembatinib for 24 h, cell lysates were collected for western blot analysis. p, phosphorylation; C, control.

### DETAILED DESCRIPTION OF THE INVENTION

### I. Definitions

In accordance with the present disclosure and as used herein, the following terms are defined with the following meanings, unless explicitly stated otherwise. Unless otherwise defined, all terms of art, notations and other scientific or medical terms or terminology used herein are intended to have the meanings commonly understood by those of skill in the chemical and medical arts.

As used herein, “TKI” is short for tyrosine kinase inhibitor, which is a pharmaceutical agent that inhibits one or more tyrosine kinase(s). Tyrosine kinases are important mediators of signaling cascades, determining key roles in diverse biological processes like growth, differentiation, metabolism and apoptosis in response to external and internal stimuli. The role of tyrosine kinases in the pathophysiology of diseases like cancer have been discovered and implicated (Manash K. Paul et al., Int J Med Sci., 2004; 1(2): 101-115). Accordingly, TKIs are typically used as anti-cancer agents. For example, TKIs have substantially improved outcomes in chronic myelogenous leukemia. Tyrosine kinases are primarily classified as receptor tyrosine kinase (RTK) e.g., EGFR, PDGFR, FGFR and the IR and non-receptor tyrosine kinase (NRTK) e.g., SRC, ABL, FAK and Janus kinase. As used herein, “multi-targeted TKI” refers to a tyrosine kinase inhibitor that has the capacity to inhibit at least two tyrosine kinases.

The term “biomarker” as used here refers to a biological molecule that is a measurable indicator of some biological state or condition. The term “biomarker” used herein is intended to encompass a polynucleotide of interest, or a polypeptide (for example encoded by the polynucleotide of interest). Examples of biomarker as provided herein can be a gene (e.g., genomic DNA, cDNA) or a product of the gene such as an mRNA transcribed from the gene, and a protein encoded by the gene. Specific examples of the biomarkers as provided herein include SDH (including SDHA, SDHB, SDHC and SDHD).

The term “level” with respect to a biomarker refers to the amount or quantity of the biomarker of interest present in a sample. Such amount or quantity may be expressed in the absolute terms, i.e., the total quantity of the biomarker in the sample, or in the relative terms, i.e., the concentration or percentage of the biomarker in the sample. Level of a biomarker can be measured at DNA level (for example, as represented by the amount or quantity or copy number of the gene in a chromosomal region), at RNA level (for example as mRNA amount or quantity), or at protein level (for example as protein or protein complex amount or quantity).

The term “activity” with respect to a biomarker refers to the biological activity (e.g., catalytic or regulatory ability) of the proteins as described here.

As used herein, “likelihood” and “likely” with respect to response of a subject to a treatment is a measurement of how probable the therapeutic response is to occur in the subject. It may be used interchangeably with “probability”. Likelihood refers to a probability that is more than speculation, but less than certainty. Thus, a therapeutic response is likely if a reasonable person using common sense, training or experience concludes that, given the circumstances, a therapeutic response is probable. In one embodiment, the term “likelihood” and “likely” denotes a chance in percent of how probable a therapeutic response is to occur. In some embodiments, a subject with cancer identified as “likely to respond” refers to a subject with cancer who has more than 30% chance, more than 40% chance, more than 50% chance, more than 60% chance, more than 70% chance, more than 80% chance, more than 90% chance of responding to a treatment.

The term “responsive” or “responsiveness” as used in the context of a subject's therapeutic response to a cancer therapy, are used interchangeably and refer to a beneficial response of a subject to a treatment as opposed to unfavorable responses, i.e., adverse events. In a subject, beneficial response can be expressed in terms of a number of clinical parameters, including loss of detectable tumor (complete response), decrease in tumor size and/or cancer cell number (partial response), tumor growth arrest (stable disease), enhancement of anti-tumor immune response, possibly resulting in regression or rejection of the tumor; relief, to some extent, of one or more symptoms associated with the tumor; increase in the length of survival following treatment; and/or decreased mortality at a given point of time following treatment. Continued increase in tumor size and/or cancer cell number and/or tumor metastasis is indicative of lack of beneficial response to treatment, and therefore decreased responsiveness.

As used herein, “cancer” or “tumor” can be used interchangeably, which is a generic name for a wide range of cellular malignancies characterized by unregulated growth, lack of differentiation, and the potential or ability to invade local tissues and metastasize. These neoplastic malignancies affect, with various degrees of prevalence, every tissue and organ in the body, and can be benign or malignant. Cancer involves presence of cells possessing characteristics typical of cancer-causing cells, such as uncontrolled proliferation, immortality, metastatic potential, rapid growth and proliferation rate, and certain characteristic morphological features. Often, cancer cells will be in the form of a solid tumor, but such cells may exist alone or may circulate in the blood stream as independent cells, such as leukemic cells.

As used herein, a “pharmaceutically acceptable” component is one that is suitable for use with humans and/or animals without undue adverse side effects (such as toxicity, irritation, and allergic response) commensurate with a reasonable benefit/risk ratio.

The terms “determining”, “measuring” and “detecting” can be used interchangeably and refer to both quantitative and semi-quantitative determinations or qualitative determinations.

The term “hybridizing” refers to the binding, duplexing or pairing of at least partially complementary strands of nucleic acid molecules. A nucleic acid strand can specifically hybridize to a target nucleic acid strand when there is sufficient degree of complementarity to avoid non-specific binding to non-target nucleic acid sequences.

The term “nucleic acid” and “polynucleotide” are used interchangeably and refer to a polymeric form of nucleotides of any length, either deoxyribonucleotides or ribonucleotides, or analogs thereof. Non-limiting examples of polynucleotides include a gene, a gene fragment, exons, introns, messenger RNA (mRNA), transfer RNA, ribosomal RNA, ribozymes, cDNA, shRNA, single-stranded short or long RNAs, recombinant polynucleotides, branched polynucleotides, plasmids, vectors, isolated DNA of any sequence, control regions, isolated RNA of any sequence, nucleic acid probes, and primers. The nucleic acid molecule may be linear or circular.

The term “complementarity” refers to the ability of baseparing between a nucleic acid sequence and another nucleic acid sequence via either traditional Watson-Crick or other non-traditional types. Complementarity can be partial or total. Partial complementarity occurs when one or more nucleic acid bases is not matched according to the base pairing rules. A percent complementarity indicates the percentage of nucleic acid base in a nucleic acid molecule which can form basepairs (e.g., Watson-Crick base pairing) with a second nucleic acid sequence (e.g., 5, 6, 7, 8, 9, 10 base pairing out of 10 bases being 50%, 60%>, 70%>, 80%>, 90%, and 100% complementary).

The term “prognosis” as used herein refers to the prediction or forecast of the future course or outcome of a disease or condition.

In general, a “protein” is a polypeptide (i.e., a string of at least two amino acids linked to one another by peptide bonds). Proteins may include moieties other than amino acids (e.g., may be glycoproteins) and/or may be otherwise processed or modified. Those of ordinary skill in the art will appreciate that a “protein” can be a complete polypeptide chain as produced by a cell (with or without a signal sequence), or can be a functional portion thereof. Those of ordinary skill will further appreciate that a protein can sometimes include more than one polypeptide chain, for example linked by one or more disulfide bonds or associated by other means.

The term “treating” or “treatment” of a condition as used herein includes preventing or alleviating a condition, slowing the onset or rate of development of a condition, reducing the risk of developing a condition, preventing or delaying the development of symptoms associated with a condition, reducing or ending symptoms associated with a condition, generating a complete or partial regression of a condition, curing a condition, or some combination thereof. Especially for cancers, the term “treating” or “treatment” of cancer as used herein, unless otherwise indicated, means reversing, alleviating, inhibiting the progress of, or preventing, either partially or completely, the growth of tumors, tumor metastases, or other cancer-causing or neoplastic cells in a subject.

As used herein, “combination therapy” is understood as administration of two or more active agents using separate formulations or a single pharmaceutical formulation, or consecutive administration in any order such that, there is a time period while both (or all) active agents simultaneously exert their biological activities. It is contemplated herein that one active agent (e.g., a multi-targeted TKI) can improve the activity of a second agent, for example, can sensitize target cells, e.g., cancer cells, to the activities of the second agent. Combination therapy does not require that the agents are administered at the same time, at the same frequency, or by the same route of administration.

The terms “administer”, “administering” or “administration” include any method of delivery of a pharmaceutical composition or agent into a subject's system or to a particular region in or on a subject. The administration may be local or systemic. In certain embodiments, the administration is by oral route or parental route (e.g., intravenous, intramuscular, subcutaneous, intramedullary, intrathecal, intraventricular, intraperitoneal, intranasal, or intraocular injection; direct injection to a tumor; or intravenous infusion). In certain embodiments, routes of administration include pulmonary administration, suppositories, and transdermal or transcutaneous applications. Administering an agent can be performed by a number of people working in concert. Administering an agent includes, for example, prescribing an agent to be administered to a subject and/or providing instructions, directly or through another, to take a specific agent, either by self-delivery, e.g., as by oral delivery, subcutaneous delivery, intravenous delivery through a central line, etc.; or for delivery by a trained professional, e.g., intravenous delivery, intramuscular delivery, intratumoral delivery, continuous infusion, etc.

As used herein, the term “subject” refers to a human or any non-human animal or mammal (e.g., mouse, rat, rabbit, dog, cat, cattle, swine, sheep, horse or primate). In many embodiments, a subject is a human being. A subject can be a patient, which refers to a human presenting to a medical provider for diagnosis or treatment of a disease. The term “subject” is used herein interchangeably with “individual” or “patient.” A subject can be afflicted with or is susceptible to a disease or disorder but may or may not display symptoms of the disease or disorder.

The term “therapeutically effective amount” or “effective amount” means the amount of a compound that that produces some desired local or systemic therapeutic effect at a reasonable benefit/risk ratio applicable to any treatment. When administered for preventing a disease, the amount is sufficient to avoid or delay onset of the disease. A therapeutically effective amount or an effective amount need not be curative or prevent a disease or condition from ever occurring. In certain embodiments, a therapeutically-effective amount of a compound will depend on its therapeutic index, solubility, and the like.

In all occurrences in this application where there are a series of recited numerical values, it is to be understood that any of the recited numerical values may be the upper limit or lower limit of a numerical range. It is to be further understood that the invention encompasses all such numerical ranges, i.e., a range having a combination of an upper numerical limit and a lower numerical limit, wherein the numerical value for each of the upper limit and the lower limit can be any numerical value recited herein. Ranges provided herein are understood to include all values within the range. For example, 1-10 is understood to include all of the values 1, 2, 3, 4, 5, 6, 7, 8, 9, and 10, and fractional values as appropriate. Similarly, ranges delimited by “at least” are understood to include the lower value provided and all higher numbers.

As used herein, “about” a value or parameter herein includes (and describes) embodiments that are directed to that value or parameter per se. For example, description referring to “about X” includes description of “X”. Numeric ranges are inclusive of the numbers defining the range. Generally speaking, the term “about” refers to the indicated value of the variable and to all values of the variable that are within the experimental error of the indicated value (e.g., within the 95% confidence interval for the mean) or within 10 percent of the indicated value, whichever is greater. Where the term “about” is used within the context of a time period (years, months, weeks, days etc.), the term “about” means that period of time plus or minus one amount of the next subordinate time period (e.g. about 1 year means 11-13 months; about 6 months means 6 months plus or minus 1 week; about 1 week means 6-8 days; etc.), or within 10 percent of the indicated value, whichever is greater.

The term “including” is used herein to mean, and is used interchangeably with, the phrase “including but not limited to”. Similarly, “such as” is used herein to mean, and is used interchangeably, with the phrase “such as but not limited to”.

The term “or” is used inclusively herein to mean, and is used interchangeably with, the term “and/or,” unless context clearly indicates otherwise.

### II. Methods for Treating a Disease, Disorder or Condition Associated with SDH-Deficiency

Described herein are a method of treating a disease, disorder or condition associated with SDH-deficiency in a subject, and a method of predicting likelihood of responsiveness of a subject in need thereof to the treatment of a multi-targeted TKI.

Numerous TKIs aiming at various tyrosine kinases have been generated by the originators of these compounds and proven to be effective anti-tumor agents and anti-leukemic agents (e.g., Anafi M et al., Blood, 1993, 82 (12): 3524-9). Commercial TKIs including, for example, imatinib, gefitinib, erlotinib, dasatinib, etc. have been developed against cancers like GIST, the most common mesenchymal tumors of the gastrointestinal tract. Prior to the advent of TKIs, there were few treatment options available to patients with metastatic GIST. Nevertheless, treatment resistance is a formidable challenge in managing locally advanced or metastatic GIST. It has been reported that patients with SDH-deficient GIST are not sensitive to imatinib and other TKIs. As surprisingly discovered by the inventors, subjects with TKI-resistant and SDH-deficient GIST are likely to respond to treatment of the multi-targeted TKI as provided herein (e.g., olverembatinib).

In one aspect, the present disclosure provides a method of treating a disease, disorder or condition associated with SDH-deficiency in a subject, comprising administering a therapeutically effective amount of a TKI to the subject, thereby treating the disease, disorder or condition associated with SDH-deficiency in the subject.

In another aspect, the present disclosure provides a method of identifying a subject having a disease, disorder or condition associated with succinate dehydrogenase- (SDH-) deficiency and selecting a treatment for the identified subject, comprising: a) determining if a biological sample from the subject is SDH-deficient; b) identifying the subject as having the disease, disorder or condition associated with SDH-deficiency, based on the SDH-deficiency determined in step a), and c) recommending the subject to receive treatment of a multi-targeted TKI.

### 2.1 SDH-Deficiency

In some embodiments, the subject as provided herein is determined or has been determined as having SDH-deficiency.

As used herein, “SDH” is short for succinate dehydrogenase, which is also known as succinate-coenzyme Q reductase (SQR) or respiratory complex II. The term “SDH” is intended to encompass the SDH gene (e.g., genomic DNA, cDNA), as well as the SDH gene product (e.g., mRNA transcribed from the gene, a protein encoded by the gene). SDH is an enzyme complex found in many bacterial cells and in the inner mitochondrial membrane of eukaryotes. It couples the oxidation of succinate to fumarate in the citric acid cycle, as well as the reduction of ubiquinone to ubiquinol in the electron transport chain, participating in both the citric acid cycle and the electron transport chain (James MacFarlane, et al., Clinical Endocrinology, (2020) 93:528-538).

Mitochondrial and many bacterial SDHs are composed of four structurally different subunits: two hydrophilic and two hydrophobic. The first two subunits, a flavoprotein (SDHA) and an iron-sulfur protein (SDHB), form a hydrophilic head where enzymatic activity of the complex takes place. The second two subunits are hydrophobic membrane anchor subunits, SDHC and SDHD.

Exemplary sequence of human SDHA is available in UniProtKB database under the accession number of P31040 (SDHA_HUMAN), in the GenBank database under the Gene ID of 6389. Exemplary sequence of human SDHB is available in UniProtKB database under the accession number of P21912 (SDHB_HUMAN), in the GenBank database under the Gene ID of 6390. Exemplary sequence of human SDHC is available in UniProtKB database under the accession number of Q99643 (C560_HUMAN), in the GenBank database under the Gene ID of 6391. Exemplary sequence of human SDHB is available in UniProtKB database under the accession number of O14521 (DHSD_HUMAN), in the GenBank database under the Gene ID of 6392.

As used herein, “deficiency” or “deficient” with respect to a biomarker (such as SDH) refers to insufficiency in activity or level of the biomarker, and can include, for example, being less than normal activity or level, or being absent or null in activity or level of the biomarker. The term “SDH-deficiency” or “SDH-deficient” as used herein refers to insufficiency in level or activity of SDH in the biological sample. In certain embodiments, “SDH-deficiency” or “SDH-deficient” causes reduction in SDH enzyme activity, reduction in SDH expression, or destabilization of SDH protein complex.

In certain embodiments, the methods as provided herein comprise determining in the biological sample if there is SDH-deficiency.

In some embodiments, the SDH-deficiency is indicated by presence of one or more loss of function mutations in SDHA, SDHB, SDHC, SDHD or any combination thereof. Accordingly, the methods as provided herein can comprise the step of detecting the presence of one or more loss of function mutations in SDHA, SDHB, SDHC, SDHD or any combination thereof in the biological sample.

The term “loss of function mutation” as used herein with respect to a biomarker, refers to a mutation that results in at least partial (or complete) loss of function or activity of the gene or of the gene product of the biomarker, or results in a nonfunctional gene or gene product of the biomarker, or results in decreased (or completely no) gene expression (e.g., decreased mRNA or protein abundance in SDHA, SDHB, SDHC or SDHD). The activity and/or level of the affected gene or gene product of the biomarker would be significantly lower than wild-type counterpart or even be eliminated. A loss of function in the SDH enzyme complex is most commonly caused by an inherited mutation in one of the four SDHx genes (SDHA, SDHB, SDHC and SDHD).

In some embodiments, the loss of function mutation could be missense substitution, deletion, insertion, translocation, and premature stop codon, or any combination thereof.

The loss of function mutation can be caused by germline oncogenic missense mutations in SDHB that affect amino acid residues that are important for Fe—S cluster acquisition (L(I)YR motif amino acid residues) or ligation (cysteines) (Neetu Saxena et al., JNCI J Natl Cancer Inst, (2016) 108 (1): djv287).

As used herein, a “missense substitution” is a mutation that exchanges one nucleobase for another in a polynucleotide sequence, or that substitutes one amino acid residue for another in a polypeptide sequence. Such substitution in a polynucleotide sequence can change a codon to one that encodes a different amino acid residue, and therefore will cause change in amino acid sequence in the protein produced.

As used herein, a “deletion” is a mutation in which one or more nucleobase pairs are lost or deleted from a polynucleotide sequence, or in which one or more amino acid residue are deleted from a polypeptide sequence.

As used herein, an “insertion” is a mutation in which one or more extra nucleobase pairs are inserted into a place in a polynucleotide sequence, or in which one or more amino acid residue is inserted into a polypeptide sequence.

In certain embodiments, insertion or deletion in a polynucleotide sequence may cause frame shift, which changes the reading frame of the codons and results in a completely different translated gene product from the original. This often generates truncated proteins that result in loss of function.

As used herein, a “translocation” is a genetic change in which a piece of one chromosome breaks off and attaches to another chromosome. Sometimes pieces from two different chromosomes will trade places with each other.

As used herein, a “premature stop codon” is a mutation present in mRNA prior to their normal position in the gene. This means that the message to create the protein of interest is incomplete; thus only incomplete (commonly referred to as truncated) protein is formed.

In some embodiments, the one or more loss of function mutations comprise mutations as indicated in the expressed proteins. The exemplary mutations which are known in the art include but are not limited to the following.

SDHA: p.G184R and p.P279S.

SDHB: p.A15T, p.W218Ter, SDHB KO exon 2+3 (Heterozygote), p.I44N, p.R46G, p.R46Q, p.C93R, p.C93Y, p.C98R, p.C98Y, p.C101Y, p.C113Y, p.C186Y, p.C189R, p.C189F, p.C191Y, p.C192R, p.C192Y, p.C196Y, p.C196W, p.R242S, p.R242C, p.R242H, p.C243S, p.C249G, p.C249Y and p.C253Y (see for example, Neetu Saxena et al., JNCI J Natl Cancer Inst (2016) 108(1): djv287).

In some embodiments, the one or more loss of function mutations comprise mutations as indicated in the nucleic acid sequence (see for example, Ahmad Ibrahim and Shefali Chopra, Arch Pathol Lab Med (2020) 144 (5): 655-660). The exemplary mutations which are known in the art include but are not limited to the mutations as listed in Table 1.

TABLE 1
Mutations in SDH

| Gene | Mutation |
|---|---|
| SDHA | c.91C > T, c.113A > T, c.160C > T, c.511C > T, c.533C > T, |
| | c.457-3_457-1delCAG, c.562C > T, c.628C > T, c.666del, |
| | c.688delG, c.767C > T, c.800C > T, c.818C > T, c.923C > T, |
| | c.985C > T, c.1043-1055del, c.1046_1047delTG, c.1054C > T, |
| | c.1151C > G, c.1215_1216insCC, c.1255G > A, c.1334C > T, |
| | c.1352G > A, c.1360G > A, c.1361C > A, c.1367C > T, |
| | c.1468delG, c.1513delA, c.1532T > C, c.1534C > T, |
| | c.1753C > T, c.1765C > T, c.1766G > A, c.1690G > A, |
| | c.1794G > C, c.1795-1G > T, c.1798C > T, c.1799G > A |
| SDHB | c.17_42dup, c.71_72insA, c.136C > T, c.137G > A, |
| | c.274T > A, c.301delT, c.380T > G, c.423 + 1G > A, |
| | c.423 + 20T > A, c.566G > T, c.600G > T, c.643-1G > C, |
| | c.725G > A, c.778_779insG |
| SDHC | c.1A > G, c.6delT, c.65G > A, c.160C > A, c.221A > G, |
| | c.224G > A, c.238C > T, c.301delT, c.380A > G, c.397C > T, |
| | c.405 + 1G > A |
| SDHD | c.34G > A, c.352delG, c.416T > C, c.455G > A |

It is to be understood that the present disclosure is not limited to any specific SDH mutations. Any loss of function mutations in SDH can be useful in the present disclosure. The mutations as provided herein are denoted according to common rules in the art (see for example, Ahmad Ibrahim and Shefali Chopra, Arch Pathol Lab Med (2020) 144 (5): 655-660), and could be clearly understood by a person having ordinary skill in the art.

Provided below is a simplified explanation for easy reference and not intended to make any limitations.

The missense substitution can be denoted as p.AnB, where “n” is a number indicating the n^{th} amino acid residue in the polypeptide sequence, “A” is the amino acid residue at the n^{th} residue in the wild-type polypeptide sequence, and “B” is the mutated amino acid residue at the n^{th} residue. For instance, “p.A15T” denotes that the 15^{th} amino acid residue Ala (A) is changed to a Threonine (T).

The premature stop codon can be denoted as “Ter” after the terminal amino acid residue of the incomplete protein. For instance, “p. W218Ter” denotes that the translation abnormally terminated after 218th amino acid residue Tryptophan (W), and the 218^{th} amino acid residue Tryptophan (W) is the c-terminal amino residue of the obtained incomplete protein.

As shown in Table 1, substitution in a nucleic acid sequence can be denoted as c.nA>B, where “n” is a number indicating the n^{th} nucleotide in the nucleic acid sequence, “A” is the nucleotide at the n^{th} residue in the wild-type nucleic acid sequence, and “B” is the mutated nucleotide at the n^{th} nucleotide. For instance, “c.91C>T” denotes that the 91^{th} nucleotide C is changed to a T.

As shown in Table 1, deletion in a nucleic acid sequence can be denoted by “del” after the nucleotide number(s) indicating the deletion site. For instance, “c. 1043-1055del” denotes that nucleotides 1043-1055 are deleted. Optionally the “del” is followed by one or more nucleotide(s), indicating the nucleotide(s) deleted from the given deletion site(s).

As shown in Table 1, duplication of one or more nucleotide(s) in a nucleic acid sequence can be denoted as “dup” after the nucleotide number(s) indicating the nucleotide(s) that are duplicated. For example, “c.17_42dup” denotes a duplication of 26 nucleotides at positions 17 to 42 occurs.

As shown in Table 1, insertion in a nucleic acid sequence can be denoted as “ins” after the nucleotide number(s) flanking the insertion site, followed by the nucleotide(s) inserted. For instance, “c.1215_1216insCC” denotes that CC are inserted between nucleotides 1215 and 1216.

In some embodiments, the loss of function mutation can be a single-copy or two-copy mutation. Thus, the mutation can be loss of function even if it only causes haploinsufficiency. Accordingly, the loss of function mutation can have a homozygous deletion mutation of SDH, a deletion mutation of one allele, a deletion mutation of one allele and a point mutation of another allele of SDH, or heterozygous mutations of SDH.

In some embodiments, the SDH-deficiency is indicated by SDHC promoter methylation. Accordingly, the methods as provided herein can comprise the step of detecting the presence of SDHC promoter methylation in the biological sample.

To determine if there is SDH-deficiency, the methods as provided herein can comprise the step of determining if promoter of SDHC is negatively alternated in such a way that would lead to reduced expression of SDHC. In certain embodiments, the methods of the present disclosure include determining if promoter of SDHC is hypermethylated in the biological sample, wherein hypermethylation in the promoter is indicative of the SDH-deficiency.

It has been reported that about 75% to 80% of patients with GIST have mutations in the KIT gene, while 5% to 10% have mutations in the platelet-derived growth factor receptor a (PDGFRA) gene (Vallilas C et al., Int J Mol Sci, 2021; 22 (2): 493). Mutations of 707 driver genes have been identified, including oncogenes (OG) and tumor suppressor genes (TSG), and a fusion gene (F) (Keiichi Ohshima et al., Cancer Sci. 2019 December; 110 (12): 3821-3833). The subject having SDH-sufficiency as provided herein may also bear other mutations in genes. In some embodiments, the subject (e.g., the biological sample of the subject) having SDH-sufficiency further comprises one or more of wild-type KIT, wild-type PDGFR, or combination thereof. In some embodiments, the subject having SDH-sufficiency further comprises wild-type KIT. In some embodiments, the subject (e.g., the biological sample of the subject) having SDH-sufficiency further comprises one or more mutations in NF1/BRAF/RAS pathway genes, PIK3CA, or fusions affecting NTRK3, or any combination thereof. In some embodiments, the subject (e.g., the biological sample of the subject) has been determined as overexpressing VEGFR or FGF ligand.

In some embodiments, the SDH-deficiency is determined in a biological sample from the subject.

Any biological sample suitable for conducting the methods as provided herein can be obtained from the subject. As used herein, “biological sample” refers to a biological specimen taken by sampling from a subject, optionally with additional processing. In certain embodiments, the biological sample could be any biological samples that can be used for detection of germline mutation of SDH genes.

In some embodiments, the biological sample can be any fresh or archived sample containing cancer cells or non-cancer cells obtained from a tumor tissue, e.g., by a tumor biopsy or fine needle aspirate. In some embodiments, the sample can be any biological fluid containing cancer cells or non-cancer cells. The collection of a sample from a subject is performed in accordance with the standard protocol generally followed by hospital or clinics, such as during a biopsy. Examples of a biological sample include but are not limited to, tissues, such as biopsy tissue (e.g., biopsied bone tissue, bone marrow, breast tissue, gastroinstetinal tract tissue, lung tissue, liver tissue, prostate tissue, brain tissue, nerve tissue, meningeal tissue, renal tissue, endometrial tissue, cervical diffuse, lymph node tissue, muscle tissue, or skin tissue), a frozen tissue, a paraffin embedded tissue; and fluid, such as blood, plasma, serum, peripheral blood mononuclear cells (PBMC), urine, vaginal fluid, uterine or vaginal flushing fluids, plural fluid, ascetic fluid, cerebrospinal fluid, saliva, sweat, tears, sputum, bronchioalveolar lavage fluid, etc. In a further embodiment, a biological sample comprises cells, tissue (e.g., tumor tissue), whole blood, plasma, serum, PBMC, urine, mouthwash, stool, saliva, and any combination thereof. In a further embodiment, a biological sample is tissue (e.g., tumor tissue), whole blood, plasma, or PBMC. In a preferred embodiment, a biological sample is tumor tissue.

In certain embodiments, the sample can be further processed by a desirable method for determining the genetic status, activity or level of the at least one biomarker (such as SDH).

In certain embodiments, the method further comprises isolating or extracting cancer cell (such as circulating tumor cell) from the biological tissue sample or the fluid sample (such as PBMC) obtained from the subject. The cancer cells can be separated by immunomagnetic separation technology such as that available from Immunicon (Huntingdon Valley, Pa.).

In certain embodiments, a tissue sample can be processed to perform in situ hybridization. For example, the tissue sample can be paraffin-embedded before fixing on a glass microscope slide, and then deparaffinized with a solvent, typically xylene.

In certain embodiments, the method further comprises isolating the nucleic acid from the sample, if RNA or DNA level of the biomarker is to be measured. Various methods of extraction are suitable for isolating the DNA or RNA from cells or tissues, such as phenol and chloroform extraction, and various other methods as described in, for example, Ausubel et al., Current Protocols of Molecular Biology (1997) John Wiley & Sons, and Sambrook and Russell, Molecular Cloning: A Laboratory Manual 3^{rd} ed. (2001).

Commercially available kits can also be used to isolate RNA, including for example, the NucliSens extraction kit (Biomerieux, Marcy l′Etoile, France), QIAamp™ mini blood kit, Agencourt Genfind™, Rneasy® mini columns (Qiagen), PureLink® RNA mini kit (Thermo Fisher Scientific), and Eppendorf Phase Lock Gels™. A skilled person can readily extract or isolate RNA or DNA following the manufacturer's protocol.

In some embodiments, the SDH-deficiency is determined by immunohistochemistry, western blot, amplification-based assay, hybridization-based assay, and sequencing.

Mutation status of a biomarker (e.g., SDH as provided herein) at DNA or RNA level can be measured by any methods known in the art, for example, without limitation, an amplification assay, a hybridization assay, or a sequencing assay. Mutation status at protein level can be measured by any methods known in the art, for example, without limitation, immunoassays. In certain embodiments, a loss of SDHB staining by immunohistochemistry can be used for diagnosing pathogenic germline variants in the SDHx genes and can provide a rapid assessment of pathogenicity for a novel SDHx variant of uncertain significance (Papathomas T G, et al., Mod Pathol, 2015; 28(6):807-821).

Expression level of a biomarker (e.g., SDH as provided herein) at DNA or RNA level can be measured by any methods known in the art, for example, without limitation, an amplification assay, a hybridization assay, or a sequencing assay. Expression level of a biomarker (e.g., SDH as provided herein) at protein level can be measured by any methods known in the art, for example, without limitation, immunoassays.

Activity level of a biomarker can be measured by a suitable functional assay known in the art, for example, without limitation, by a phosphorylation assay.

These methods are well-known in the art, and are described in detail below as exemplary illustration.

### (a) Amplification Assay

A nucleic acid amplification assay involves copying a target nucleic acid (e.g., DNA or RNA), thereby increasing the number of copies of the amplified nucleic acid sequence. Amplification may be exponential or linear. Exemplary nucleic acid amplification methods include, but are not limited to, amplification using the polymerase chain reaction (“PCR”, see U.S. Pat. Nos. 4,683,195 and 4,683,202; PCR Protocols: A Guide To Methods And Applications (Innis et al., eds, 1990)), reverse transcriptase polymerase chain reaction (RT-PCR), quantitative real-time PCR (qRT-PCR); quantitative PCR, such as TaqMan®, nested PCR, ligase chain reaction (Abravaya, K., et al., Nucleic Acids Research, 23:675-682, (1995), branched DNA signal amplification (Urdea, M. S., et al., AIDS, 7 (suppl 2):S11-S14, (1993), amplifiable RNA reporters, Q-beta replication (Lizardi et al., Biotechnology (1988) 6:1197), transcription-based amplification (Kwoh et al., Proc. Natl. Acad. Sci. USA (1989) 86:1173-1177), boomerang DNA amplification, strand displacement activation, cycling probe technology, self-sustained sequence replication (Guatelli et al., Proc. Natl. Acad. Sci. USA (1990) 87:1874-1878), rolling circle replication (U.S. Pat. No. 5,854,033), isothermal nucleic acid sequence based amplification (NASBA), and serial analysis of gene expression (SAGE).

In some embodiments, to measure the mRNA level of the biomarker, the target RNA of the biomarker is reverse transcribed to cDNA before the amplification. Various reverse transcriptases may be used, including, but not limited to, MMLV RT, RNase H mutants of MMLV RT such as Superscript and Superscript II (Life Technologies, GIBCO BRL, Gaithersburg, Md.), AMV RT, and thermostable reverse transcriptase from Thermus thermophilus. For example, one method which may be used to convert RNA to cDNA is the protocol adapted from the Superscript II Preamplification system (Life Technologies, GIBCO BRL, Gaithersburg, Md.; catalog no. 18089-011), as described by Rashtchian, A., PCR Methods Applic., 4: S83-S91, (1994).

In certain embodiments, the expression level of RNA (e.g., mRNA) or the copy number variation of DNA of the biomarkers is quantified after the nucleic acid amplification assay. For example, the amplified products can be separated on an agarose gel and stained with ethidium bromide followed by detection and quantification using standard gel electrophoresis methods. Alternatively, the amplified products can be integrally labeled with a suitable detectable label (e.g. a radio- or fluorescence nucleotide) and then visualized using x-ray film or under the appropriate stimulating spectra.

In certain embodiments, the expression level of RNA (e.g. mRNA) or the copy number variation of DNA of the biomarkers is quantified during the nucleic acid amplification assay, which is also known as real-time amplification or quantitative amplification. Methods of quantitative amplification are disclosed in, e.g., U.S. Pat. Nos. 6,180,349; 6,033,854; and 5,972,602, as well as in, e.g., Gibson et al., Genome Research (1996) 6:995-1001; DeGraves, et al., Biotechniques (2003) 34(1): 106-10, 112-5; Deiman B, et al., Mol Biotechnol. (2002) 20(2): 163-79. Quantification is usually based on the monitoring of the detectable signal representing copies of the template in cycles of an amplification (e.g., PCR) reaction. Detectable signals can be generated by intercalating agents (e.g., SYBR GREEN™ and SYBR GOLD™) or labeled primer or labeled probes used during the amplification.

In certain embodiments, the labeled primer or labeled probe comprise a detectable label comprising a fluorophore. In certain embodiments, the labeled primer or labeled probe may further comprise a quencher substance. Presence of both a fluorophore and a quencher substance (“dual labeled”) in one primer or probe could be helpful to provide for a self-quenching probe such as a TaqMan (U.S. Pat. Nos. 5,210,015 and 5,538,848) or Molecular Beacon probe (U.S. Pat. Nos. 5,118,801 and 5,312,728), or other stemless or linear beacon probe (Livak et al., 1995, PCR Method Appl., 4:357-362; Tyagi et al, 1996, Nature Biotechnology, 14:303-308; Nazarenko et al., 1997, Nucl. Acids Res., 25:2516-2521; U.S. Pat. Nos. 5,866,336 and 6,117,635). In an intact primer or probe, the quencher substance and the fluorophore are in close proximity, such that when the fluorophore is excited by irradiation, it transfers energy to the quencher substance in the same probe via fluorescence resonance energy transfer (FRET), thereby does not emit a signal.

In a quantitative amplification assay (such as real-time PCR), the expression level of RNA (e.g. mRNA) or the copy number variation of DNA of the biomarkers can be quantified using methods known in the art. For example, during the amplification, the fluorescence signal can be monitored and calculated during each PCR cycle. The threshold cycle, or Ct value can be further calculated. Ct value is the cycle at which fluorescence intersects a predetermined value. The Ct can be correlated to the initial amount of nucleic acids or number of starting cells using a standard curve. A standard curve is constructed to correlate the differences between the Ct values and the logarithmic level of the measured biomarker.

As a quality control measure, the expression level or copy number variation of an internal control biomarker may be measured. The skilled artisan will understand that an internal control biomarker can be inherently present in the sample and its expression level or copy number variation can be used to normalize the measured expression level or copy number variation of the biomarkers of interest, to offset any difference in the absolute amount of the sample.

### (b) Hybridization Assay

Nucleic acid hybridization assays use probes to hybridize to the target nucleic acid, thereby allowing detection of the target nucleic acid. Non-limiting examples of hybridization assay include Northern blotting, Southern blotting, in situ hybridization, microarray analysis, and multiplexed hybridization-based assays.

In certain embodiments, the probes for hybridization assay are detectably labeled. In certain embodiments, the nucleic acid-based probes for hybridization assay are unlabeled. Such unlabeled probes can be immobilized on a solid support such as a microarray, and can hybridize to the target nucleic acid molecules which are detectably labeled.

In certain embodiments, hybridization assays can be performed by isolating the nucleic acids (e.g. RNA or DNA), separating the nucleic acids (e.g. by gel electrophoresis) followed by transfer of the separated nucleic acid on suitable membrane filters (e.g. nitrocellulose filters), where the probes hybridize to the target nucleic acids and allows detection. See, for example, Molecular Cloning: A Laboratory Manual, J. Sambrook et al., eds., 2nd edition, Cold Spring Harbor Laboratory Press, 1989, Chapter 7. The hybridization of the probe and the target nucleic acid can be detected or measured by methods known in the art. For example, autoradiographic detection of hybridization can be performed by exposing hybridized filters to photographic film. Densitometric scanning of the photographic films exposed by the hybridized filters provides an accurate measurement of the target nucleic acid levels. Computer imaging systems can also be used to quantify the level of the biomarker.

In some embodiments, hybridization assays can be performed on microarrays. Microarrays provide a method for the simultaneous measurement of the levels of large numbers of target nucleic acid molecules. The target nucleic acids can be RNA, DNA, cDNA reverse transcribed from mRNA, or chromosomal DNA. The target nucleic acids can be allowed to hybridize to a microarray comprising a substrate having multiple immobilized nucleic acid probes arrayed at a density of up to several million probes per square centimeter of the substrate surface. The RNA or DNA in the sample is hybridized to complementary probes on the array and then detected by laser scanning. Hybridization intensities for each probe on the array are determined and converted to a quantitative value representing relative levels of the RNA or DNA. See, U.S. Pat. Nos. 6,040,138, 5,800,992 and 6,020,135, 6,033,860, and 6,344,316.

Techniques for the synthesis of these arrays using mechanical synthesis methods are described in, e.g., U.S. Pat. No. 5,384,261. Although a planar array surface is often employed the array may be fabricated on a surface of virtually any shape or even a multiplicity of surfaces. Arrays may be peptides or nucleic acids on beads, gels, polymeric surfaces, fibers such as fiber optics, glass or any other appropriate substrate, see U.S. Pat. Nos. 5,770,358, 5,789,162, 5,708,153, 6,040,193 and 5,800,992. Arrays may be packaged in such a manner as to allow for diagnostics or other manipulation of an all-inclusive device. Useful microarrays are also commercially available, for example, microarrays from Affymetrix, from Nano String Technologies, QuantiGene 2.0 Multiplex Assay from Panomics.

In certain embodiments, hybridization assays can be in situ hybridization assay. In situ hybridization assay is useful to detect the presence of copy number variation (e.g. increase or amplification) at the locus of the biomarker of interest (e.g. SDH). Probes useful for in situ hybridization assay can be locus specific probes, which hybridize to a specific locus on a chromosome to detect the presence or absence of a specific locus of interest (e.g. SDH). Other types of probes may also be useful, for example, chromosome enumeration probes (e.g. hybridizable to a repeat sequence region in a chromosomal of interest to indicate presence or absence of the entire chromosome), and chromosome arm probes (e.g. hybridizable to a chromosomal region and indicate the presence or absence of an arm of a specific chromosome). Methods for use of unique sequence probes for in situ hybridization are described in U.S. Pat. No. 5,447,841, incorporated herein by reference. Probes can be viewed with a fluorescence microscope and an appropriate filter for each fluorophore, or by using dual or triple band-pass filter sets to observe multiple fluorophores. See, e.g., U.S. Pat. No. 5,776,688 to Bittner, et al., which is incorporated herein by reference. Any suitable microscopic imaging method can be used to visualize the hybridized probes, including automated digital imaging systems. Alternatively, techniques such as flow cytometry can be used to examine the hybridization pattern of the probes.

### (c) Sequencing Methods

Sequencing methods allow determination of the nucleic acid sequence of the target nucleic acid, and can also permit enumeration of the sequenced target nucleic acid, thereby measures the level of the target nucleic acid. Examples of sequence methods include, without limitation, RNA sequencing, pyrosequencing, and high throughput sequencing.

High throughput sequencing involves sequencing-by-synthesis, sequencing-by-ligation, and ultra-deep sequencing (such as described in Marguiles et al., Nature 437 (7057): 376-80 (2005)). Sequence-by-synthesis involves synthesizing a complementary strand of the target nucleic acid by incorporating labeled nucleotide or nucleotide analog in a polymerase amplification. Immediately after or upon successful incorporation of a label nucleotide, a signal of the label is measured and the identity of the nucleotide is recorded. The detectable label on the incorporated nucleotide is removed before the incorporation, detection and identification steps are repeated. Examples of sequence-by-synthesis methods are known in the art, and are described for example in U.S. Pat. Nos. 7,056,676, 8,802,368 and 7,169,560, the contents of which are incorporated herein by reference. Sequencing-by-synthesis may be performed on a solid surface (or a microarray or a chip) using fold-back PCR and anchored primers. Target nucleic acid fragments can be attached to the solid surface by hybridizing to the anchored primers, and bridge amplified. This technology is used, for example, in the Illumina® sequencing platform.

Pyrosequencing involves hybridizing the target nucleic acid regions to a primer and extending the new strand by sequentially incorporating deoxynucleotide triphosphates corresponding to the bases A, C, G, and T (U) in the presence of a polymerase. Each base incorporation is accompanied by release of pyrophosphate, converted to ATP by sulfurylase, which drives synthesis of oxyluciferin and the release of visible light. Since pyrophosphate release is equimolar with the number of incorporated bases, the light given off is proportional to the number of nucleotides adding in any one step. The process is repeated until the entire sequence is determined.

In certain embodiments, the detection of mutation and/or wild-type status and the measurement of level of biomarkers of interest described herein is by whole transcriptome sequencing, or RNA sequencing (e.g., RNA-Seq). The method of RNA sequencing has been described (see Wang Z, Gerstein M and Snyder M, Nature Review Genetics (2009) 10:57-63; Maher C A et al., Nature (2009) 458:97-101; Kukurba K & Montgomery SB, Cold Spring Harbor Protocols (2015) 2015 (11): 951-969). In brief, mRNA extracted from a sample is reverse transcribed into cDNA and sheared into fragments. Fragments within proper length ranges are selected and ligated with sequencing adaptors, followed by amplification, sequencing, and mapping reads to a reference genome.

In certain embodiments, the CNV of a biomarker is determined using whole exome sequencing (WES). WES involves sequencing DNA exons (i.e. protein encoding regions) using high-throughput sequencing technology. More details of WES can be found, for example, in Ng S B et al, Nature. 461 (7261): 272-276 (2009), and Bao R et al, Cancer Inform. 2014; 13(Suppl 2): 67-82, which are incorporated herein to their entirety.

### (d) Immunoassays

Immunoassays typically involves using antibodies that specifically bind to the biomarker polypeptide or protein (e.g. the SDH protein as provided herein) to detect or measure the presence or level of the target polypeptide or protein. Such antibodies can be obtained using methods known in the art (see, e.g., Huse et al., Science (1989) 246:1275-1281; Ward et al, Nature (1989) 341:544-546), or can be obtained from commercial sources. Examples of immunoassays include, without limitation, Western blotting, enzyme-linked immunosorbent assay (ELISA), enzyme immunoassay (EIA), radioimmunoassay (RIA), sandwich assays, competitive assays, immunofluorescent staining and imaging, immunohistochemistry (IHC), and fluorescent activating cell sorting (FACS). The above methods are all conventional test methods in the field. For a review of immunological and immunoassay procedures, see Basic and Clinical Immunology (Stites & Terr eds., 7^{th} ed. 1991). Moreover, the immunoassays can be performed in any of several configurations, which are reviewed extensively in Enzyme Immunoassay (Maggio, ed., 1980); and Harlow & Lane, supra. For a review of the general immunoassays, see also Methods in Cell Biology: Antibodies in Cell Biology, volume 37 (Asai, ed. 1993); Basic and Clinical Immunology (Stites & Terr, eds., 7^{th} ed. 1991).

In certain embodiments, the antibodies are detectably labeled, or alternatively are not labeled but can react with a second molecule which is detectably labeled (e.g. a detectably labeled secondary antibody). Other detection systems such as time-resolved fluorescence, internal-reflection fluorescence, amplification (e.g., polymerase chain reaction) and Raman spectroscopy are also useful.

In certain embodiments, the antibodies may be immobilized on a solid substrate. The immobilization can be via covalent linking or non-covalent attachment (e.g. coating). Examples of solid substrate include porous and non-porous materials, latex particles, magnetic particles, microparticles, strips, beads, membranes, microtiter wells and plastic tubes. The choice of solid phase material and method of detectably labeling the antigen or antibody reagent are determined based upon desired assay format performance characteristics.

### (e) Activity Assays

The biological activity of a protein can be measured using a bioassay. For example, the SDH activities in a biological sample can be determined by contacting the sample with a substrate kinase (e.g. succinate) under suitable conditions, and monitoring the production of fumarate, wherein the production of fumarate indicates SDH activity. Due to a protein's ability to exert multiple biological activities, several acceptable bioassays may exist for a particular protein. Exemplary functional assays for measuring the activity of SDH can be found in for example, Andrew J. Y. Jones et al., Anal. Biochem, (2013) 442:19-23.

2.2 A Disease, Disorder or Condition Associated with SDH-Deficiency

The method as provided herein can be used for the treatment of a disease, disorder or condition associated with SDH-deficiency.

As used herein, a “disease, disorder or condition associated with SDH-deficiency” refers to a disease, disorder or condition that may be caused or exacerbated (e.g., eliciting or accelerating the onset or rate of development of a condition or symptoms associated with the condition, increasing the risk of developing a condition or symptoms associated with the condition, causing a condition or symptoms associated with the condition not responsive or less responsive to an existing therapy), in full or in part, by deficiency of SDH. In some embodiments, the “disease, disorder or condition associated with SDH-deficiency” may be identified by loss of immunohistochemical expression of SDHB (Anthony J Gill, Histopathology, (2018) 72:106-116).

In some embodiments, the disease, disorder or condition associated with SDH-deficiency could be cancer (e.g., SDH-deficient cancer). In some embodiments, the disease, disorder or condition associated with SDH-deficiency could be gastrointestinal cancer (e.g., GIST), cholangiocarcinoma, lung cancer, melanoma, breast cancer, colon cancer, ovarian cancer, prostate cancer, liver cancer (e.g., hepatocellular carcinoma), bladder cancer, pancreatic cancer, renal cancer, esophageal cancer, head and neck cancer, thyroid cancer, cutaneous squamous cell carcinoma, glioblastoma. neuroblastoma, urinary bladder cancer, hysterocarcinoma, melanoma, osteosarcoma, lymphoma (e.g., mantel cell lymphoma, diffuse large B cell lymphoma), leukemia (e.g., T-cell prolymphocytic leukemia, chronic lymphocytic leukemia, acute myeloid leukemia, acute lymphocytic leukemia (ALL)), multiple myeloma, uterine cancer, colorectal cancer, lung adenocarcinoma, uterine carcinosarcoma, lung squamous cell carcinoma, cervical cancer, esophagus cancer, sarcoma, chromophobe, renal cell carcinoma, clear cell RCC, papillary RCC, uveal melanoma, testicular germ cell tumor, low grade glioma (LGG), mesothelioma, pheochromocytoma, paraganglioma, thymoma, or adenoid cystic carcinoma (ACC).

In some embodiments, the disease, disorder or condition associated with SDH-deficiency could be SDH-deficient GIST.

In some embodiments, the disease, disorder or condition associated with SDH-deficiency could be SDH-deficient ALL, renal cell carcinoma, colon cancer, pheochromocytoma, thyroid neoplasia (e.g., papillary thyroid neoplasia, multifocal papillary thyroid neoplasia, differentiated thyroid cancer, papillary (follicular variant) thyroid neoplasia, follicular thyroid neoplasia), pancreatic neuroendocrine tumor/intrapancreatic paraganglioma, breast cancer (e.g., breast carcinoma (triple negative)), neuroblastoma, follicular lymphoma, b-cell lymphoma, Hodgkin's lymphoma, composite paraganglioma/neuroblastoma, rectal adenocarcinoma, Burkitt's lymphoma, thyroid paraganglioma, adrenocortical carcinoma, adenomatoid tumor of the adrenal gland, neuroblastoma, melanoma, synovial sarcoma, lung squamous cell carcinoma, embryonal rhabdomyosarcoma, lung adenocarcinoma, prostate adenocarcinoma, endometrial carcinoma, poorly differentiated carcinoma of the stomach, urothelial bladder cancer, colon adenocarcinoma, adenocarcinoma of the sigmoid colon, testicular seminoma, or pituitary adenoma.

### 2.3 TKI Resistance

In some embodiments, the subject as provided herein is determined or has been determined as not sensitive to at least one existing TKI.

As used herein, “not sensitive to” or “not responsive to” or “resistant to” means a patient obtains no clinical benefit or barely no clinical benefit from the prior treatment of at least one multi-targeted TKI. In some embodiments, if the subject does not obtain complete response (CR), partial response (PR), or stable disease (SD) within 6 months of the treatment of the at least one existing TKI, then the subject is defined as acquiring a primary resistance to the prior treatment of the existing TKI. In some embodiments, if the subject obtains initial response or stable disease following ≥6 months of the treatment of the at least one existing TKI and subsequent progression, then the subject is defined as acquiring an acquired resistance to the prior treatment of the existing TKI.

In some embodiments, the previously treated existing TKI could be imatinib (Novartis), ripretinib (ZAI Lab ( [symbol] )&Deciphera), avapritinib (CSTONE Pharmaceuticals ( [symbol] )&Blueprint), crenolanib (Pfizer), masitinib (AB SCIENCE), Ponatinib (Takeda), Dasatinib (BMS), nilotinib (Novartis), sunitinib (CPPI CV), Regorafenib (BAYER), sorafenib (BAYER), anlotinib (CHIATAI TIANQING ( [symbol] [symbol] )), sulfatinib (HUTCHMED ( [symbol] ), pazopanib (GSK), rogaratinib (BAYER), Infigratinib (BridgeBio), erdafitinib (JNJ), pemigatinib (Incyte), fisogatinib (Blueprint), famitinib (Hengrui ( [symbol] )), or any combinations thereof. In further embodiments, the previously treated existing TKI could be ripretinib (ZAI Lab ( [symbol] )&Deciphera), dasatinib (BMS), sunitinib (CPPI CV), regorafenib (BAYER), anlotinib (CHIATAI TIANQING ( [symbol] ), famitinib (Hengrui ( [symbol] )), or any combinations thereof.

### 2.4 Multi-Targeted TKI

The multi-targeted TKI disclosed in the present invention is capable of inhibiting at least two tyrosine kinases. Tyrosine kinases are enzymes responsible for the activation of many proteins by signal transduction cascades. The proteins are activated by adding a phosphate group to the protein (phosphorylation), a step that TKIs inhibit.

In some embodiments, the multi-targeted TKI as provided herein is capable of inhibiting at least two tyrosine kinases selected from the group consisting of: KIT, PDGFR, SRC, VEGFR (e.g., any one of VEGFR1, VEGFR2, VEGFR3 or any combination thereof), FGFR (e.g., any one of FGFR1, FGFR2, FGFR3, FGFR4 or any combination thereof), or any combination thereof. In further embodiments, the multi-targeted TKI is capable of inhibiting at least two tyrosine kinases selected from the group consisting of: KIT, PDGFR, SRC, VEGFR2, FGFR1, or any combination thereof.

In some embodiments, the multi-targeted TKI is capable of inhibiting proliferation of SDH-deficient tumor cells. The “SDH-deficient tumor cell” refers to any tumor cell line that has deficiency in SDH, or any tumor cell that is primarily isolated from a subject having deficiency in SDH, or the like. The SDH-deficient tumor cell used herein could be Jurkat clone E6-1, OS-RC-2, RKO, PC12 #5F7, or any combination thereof. In some embodiments, the multi-targeted TKI has an IC₅₀ of no more than 10 μM in inhibiting proliferation of SDH-deficient tumor cells, as determined by CCK8 assay or CTG assay. In further embodiments, the multi-targeted TKI has an IC₅₀ of no more than 5 μM, 1 μM, 500 nM, 400 nM, 300 nM, 200 nM, 150 nM, 100 nM, 50 nM, 20 nM, or 10 nM in inhibiting proliferation of SDH-deficient tumor cells, as determined by CCK8 assay or CTG assay. The CCK8 assay or CTG assay can be performed by those of ordinary skill in the art with conventional means in the art. Commercially available kits can also be used for CCK8 assay or CTG assay (e.g., Shanghai Liji Medical Technology Co., Ltd; #G7573, Promega). Those of ordinary skill in the art can readily employ the assays following the manufacturer's protocol.

In some embodiments, the multi-targeted TKI as provided herein comprises a heterocyclic alkyne benzene compound having the following formula (I):

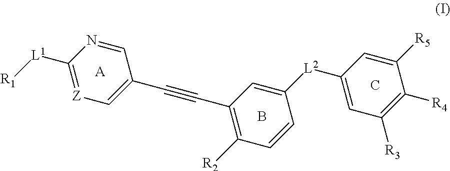

wherein Z is independently selected as CH or N; L¹ is independently selected from NH, —N═, CH; L² is independently selected as —CONH— or —NHCO—;
R₁ is independently selected from:
   1) H;
   2) C₁-C₆ alkyl;
   3) C₃-C₆ cycloalkyl;
   4) C₁-C₅ alkyl substituted by 1 or 2 hydroxy;
   5) phenyl;
   6) Groups which can fuse A ring through L¹ in Z atom site to form fused penta-heterocycles containing 1-3 N atoms and L¹ like

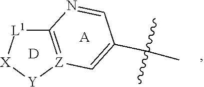

wherein, X, Y, Z are independently selected as N, CH; ring D is an aromatic heterocycle containing 1-3 N atoms;
R² is independently selected from
   1) H;
   2) Halogen;
   3) C₁-C₅ alkyl;
   4) C₃-C₆ cycloalkyl;
   5) C₁-C₅alkyl containing F;
R³ is independently selected from
   1) H;
   2) Halogen;
   3) C₁-C₄ alkyl;
   4) C₃-C₆ cycloalkyl;
   5) C₁-C₄ alkyl containing F;
when R₅ is H, R₄ is independently selected from
   1) H;
   2) (CH₂)ₙNR₆R₇;
   3) (CH₂)ₙ-Het¹;
or, when R₄ is H, R₅ is independently selected from
   1) H;
   2) Het²;
   n is independently selected from 0 or 1; Het¹ is defined as nonaromatic heterocycle containing 1-3 N atoms; Het² is defined as aromatic five-member heterocycle containing hetero atoms like N, O, and/or S; alkyl, cycloalkyl or NR₆R₇ may be incorporated into any C or N position in the nonaromatic heterocycle and aromatic heterocycle which can be substituted;
R₆ or R₇ is independently selected from:
   1) H;
   2) C₁-C₃ alkyl;
   3) C₁-C₃ alkyl containing F;
   4) C₃-C₆ cycloalkyl;
   or R₆ and R₇ can further form penta-, hexa-, hepta- or octatomic ring structure through C, O, N, S atoms;
or a pharmaceutically acceptable salt or a stereoisomer or a prodrug thereof.

In some embodiments, the multi-targeted TKI as provided herein is a compound having the following formula (II) (also known as olverembatinib, HQP1351):

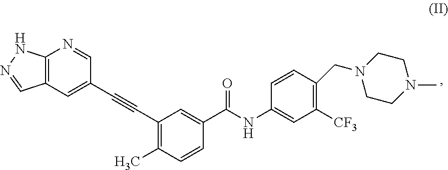 [SMILES: Cc1ccc(C(=O)Nc2ccc(CN3CCN(C)CC3)c(C(F)(F)F)c2)cc1C#Cc1cnc2[nH]ncc2c1]

or a pharmaceutically acceptable salt or a stereoisomer or a prodrug thereof.

In some embodiments, the multi-targeted TKI as provided herein is a compound selected from the group consisting of: olverembatinib, ripretinib (ZAI Lab ( [symbol] )&Deciphera), avapritinib (CSTONE Pharmaceuticals ( [symbol] )&Blueprint), crenolanib (Pfizer), masitinib (AB SCIENCE), ponatinib (Takeda), dasatinib (BMS), nilotinib (Novartis), sunitinib (CPPI CV), regorafenib (BAYER), sorafenib (BAYER), anlotinib (CHIATAI TIANQING ( [symbol] ), sulfatinib (HUTCHMED ( [symbol] ), infigratinib (BridgeBio), erdafitinib (JNJ), pemigatinib (Incyte), rogaratinib, and linsitinib, or a pharmaceutically acceptable salt or a stereoisomer or a prodrug of any of the foregoing.

More multi-targeted TKI that can be used in the present application are further disclosed in WO2012000304A1, which is incorporated herein by reference. The multi-targeted TKIs as provided herein can exist as salts.

Pharmaceutically acceptable salts of the multi-targeted TKIs as provided herein often are preferred in the methods of the invention. As used herein, the term “pharmaceutically acceptable salts” refers to salts or zwitterionic forms of the compounds of structural formula (I) or (II). Salts of compounds of formula (I) or (II) can be prepared during the final isolation and purification of the compounds or separately by reacting the compound with an acid having a suitable cation, such as, but not limited to, alkali and alkaline earth metal ions, e.g., Na⁺, K⁺, Ca²⁺, and Mg²⁺ well as organic cations such as, but not limited to, ammonium and substituted ammonium ions, e.g., NH₄⁺, NHMe₃⁺, NH₂Me₂⁺, NHMe₃⁺ and NMe₄⁺. Examples of monovalent and divalent pharmaceutically acceptable cations are discussed, e.g., in Berge et al. J. Pharm. Sci., 66:1-19 (1997).

The pharmaceutically acceptable salts of compounds of structural formula (I) or (II) can be acid addition salts formed with pharmaceutically acceptable acids. Examples of acids which can be employed to form pharmaceutically acceptable salts include inorganic acids such as nitric, boric, hydrochloric, hydrobromic, sulfuric, and phosphoric, and organic acids such as oxalic, maleic, succinic, and citric. Nonlimiting examples of salts of compounds of the invention include, but are not limited to, the hydrochloride, hydrobromide, hydroiodide, sulfate, bisulfate, 2-hydroxyethansulfonate, phosphate, hydrogen phosphate, acetate, adipate, alginate, aspartate, benzoate, bisulfate, butyrate, camphorate, camphorsulfonate, digluconate, glycerolphsphate, hemisulfate, heptanoate, hexanoate, formate, succinate, fumarate, maleate, ascorbate, isethionate, salicylate, methanesulfonate, mesitylenesulfonate, naphthylenesulfonate, nicotinate, 2-naphthalenesulfonate, oxalate, pamoate, pectinate, persulfate, 3-phenylproprionate, picrate, pivalate, propionate, trichloroacetate, trifluoroacetate, phosphate, glutamate, bicarbonate, paratoluenesulfonate, undecanoate, lactate, citrate, tartrate, gluconate, methanesulfonate, ethanedisulfonate, benzene sulphonate, and p-toluenesulfonate salts. In addition, available amino groups present in the compounds of the invention can be quaternized with methyl, ethyl, propyl, and butyl chlorides, bromides, and iodides; dimethyl, diethyl, dibutyl, and diamyl sulfates; decyl, lauryl, myristyl, and steryl chlorides, bromides, and iodides; and benzyl and phenethyl bromides. In light of the foregoing, any reference to compounds of the present invention appearing herein is intended to include compounds of structural formula (I) as well as pharmaceutically acceptable salts thereof.

Compounds having one or more chiral centers can exist in various stereoisomeric forms. Stereoisomers are compounds that differ only in their spatial arrangement. Stereoisomers include all diastereomeric, enantiomeric, and epimeric forms as well as racemates and mixtures thereof.

The term “geometric isomer” refers to cyclic compounds having at least two substituents, wherein the two substituents are both on the same side of the ring (cis) or wherein the substituents are each on opposite sides of the ring (trans). When a disclosed compound is named or depicted by structure without indicating stereochemistry, it is understood that the name or the structure encompasses one or more of the possible stereoisomers, or geometric isomers, or a mixture of the encompassed stereoisomers or geometric isomers.

When a geometric isomer is depicted by name or structure, it is to be understood that the named or depicted isomer exists to a greater degree than another isomer, that is that the geometric isomeric purity of the named or depicted geometric isomer is greater than 50%, such as at least 60%, 70%, 80%, 90%, 99%, or 99.9% pure by weight. Geometric isomeric purity is determined by dividing the weight of the named or depicted geometric isomer in the mixture by the total weight of all of the geomeric isomers in the mixture.

Racemic mixture means 50% of one enantiomer and 50% of is corresponding enantiomer. When a compound with one chiral center is named or depicted without indicating the stereochemistry of the chiral center, it is understood that the name or structure encompasses both possible enantiomeric forms (e.g., both enantiomerically-pure, enantiomerically-enriched or racemic) of the compound. When a compound with two or more chiral centers is named or depicted without indicating the stereochemistry of the chiral centers, it is understood that the name or structure encompasses all possible diasteriomeric forms (e.g., diastereomerically pure, diastereomerically enriched and equimolar mixtures of one or more diastereomers (e.g., racemic mixtures)) of the compound.

Enantiomeric and diastereomeric mixtures can be resolved into their component enantiomers or stereoisomers by well-known methods, such as chiral-phase gas chromatography, chiral-phase high performance liquid chromatography, crystallizing the compound as a chiral salt complex, or crystallizing the compound in a chiral solvent. Enantiomers and diastereomers also can be obtained from diastereomerically- or enantiomerically-pure intermediates, reagents, and catalysts by well-known asymmetric synthetic methods.

When a compound is designated by a name or structure that indicates a single enantiomer, unless indicated otherwise, the compound is at least 60%, 70%, 80%, 90%, 99% or 99.9% optically pure (also referred to as “enantiomerically pure”). Optical purity is the weight in the mixture of the named or depicted enantiomer divided by the total weight in the mixture of both enantiomers.

When the stereochemistry of a disclosed compound is named or depicted by structure, and the named or depicted structure encompasses more than one stereoisomer (e.g., as in a diastereomeric pair), it is to be understood that one of the encompassed stereoisomers or any mixture of the encompassed stereoisomers is included. It is to be further understood that the stereoisomeric purity of the named or depicted stereoisomers at least 60%, 70%, 80%, 90%, 99% or 99.9% by weight. The stereoisomeric purity in this case is determined by dividing the total weight in the mixture of the stereoisomers encompassed by the name or structure by the total weight in the mixture of all of the stereoisomers.

“Prodrug” indicates a compound that may be converted under physiological conditions or by solvolysis to a biologically active compound described herein. Thus, the term “prodrug” refers to a precursor of a biologically active compound that is pharmaceutically acceptable. A prodrug may be inactive when administered to a subject, but is converted in vivo to an active compound, for example, by hydrolysis. The prodrug compound often offers advantages of solubility, tissue compatibility or delayed release in a mammalian organism (see, e.g., Bundgard, H., Design of Prodrugs (1985), pp. 7-9, 21-24 (Elsevier, Amsterdam). A discussion of prodrugs is provided in Higuchi, T., et al., A.C.S. Symposium Series, Vol. 14, and in Bioreversible Carriers in Drug Design, ed. Edward B. Roche, American Pharmaceutical Association and Pergamon Press, 1987, both of which are incorporated in full by reference herein. The term “prodrug” is also meant to include any covalently bonded carriers, which release the active compound in vivo when such prodrug is administered to a mammalian subject. Prodrugs of an active compound, as described herein, may be prepared by modifying functional groups present in the active compound in such a way that the modifications are cleaved, either in routine manipulation or in vivo, to the parent active compound. Prodrugs include compounds wherein a hydroxy, amino or mercapto group is bonded to any group that, when the prodrug of the active compound is administered to a mammalian subject, cleaves to form a free hydroxy, free amino or free mercapto group, respectively.

### 2.5 Dosage Regimen

In some embodiments, the multi-targeted TKI as provided herein can be administered as a pharmaceutical composition. The pharmaceutical composition can comprise a multi-targeted TKI, and a pharmaceutically acceptable excipient.

As used herein, “pharmaceutically acceptable excipient” refers to a substance that aids the formulation and/or administration of an active agent to and/or absorption by a subject and can be included in the compositions of the present disclosure without causing a significant adverse toxicological effect on the subject. Non-limiting examples of pharmaceutically acceptable excipients include water, NaCl, normal saline solutions, lactated Ringer's, normal sucrose, normal glucose, binders, fillers, disintegrants, lubricants, coatings, sweeteners, flavors, salt solutions (such as Ringer's solution), alcohols, oils, gelatins, carbohydrates such as lactose, amylose or starch, hydroxymethycellulose, fatty acid esters, polyvinyl pyrrolidine, and colors, and the like. The excipients are “acceptable” in the sense of being compatible with the other ingredients of the pharmaceutical composition and not deleterious to the recipient thereof.

One skilled in the art would be able, by routine experimentation, to determine what an effective, non-toxic amount of the multi-targeted TKI as provided herein would be for the purpose of treating the disease, disorder or condition associated with SDH-deficiency as provided herein. For example, a therapeutically active amount of multi-targeted TKI may vary according to factors such as the disease stage (e.g., stage I versus stage IV), age, sex, medical complications (e.g., immunosuppressed conditions or diseases) and weight of the subject, and the ability of the multi-targeted TKI to elicit a desired response in the subject. In certain embodiments, a therapeutically active amount is a safe amount of the multi-targeted TKI which is sufficient to yield a desired therapeutic response without undue adverse side effects (such as toxicity, irritation, or allergic response) commensurate with a reasonable benefit/risk ratio when used in the manner of this disclosure.

The dosage regimen may be adjusted to provide the optimum therapeutic response. For example, several divided doses may be administered daily or administered by continuous dosing or the dose may be proportionally reduced as indicated by the exigencies of the therapeutic situation. In some embodiments, the multi-targeted TKI as provided herein is administered in an amount that would be therapeutically effective if delivered alone or in combination, i.e., the multi-targeted TKI is administered and/or acts as a therapeutic treatment of the disease, disorder or condition associated with SDH-deficiency, and not predominantly as an agent to ameliorate side effects of other treatments.

In some embodiments, the multi-targeted TKI as provided herein is administered in an amount that would be effective in the treatment of the disease, disorder or condition associated with SDH-deficiency. The dosages provided below may be used for any mode of administration of the multi-targeted TKI as provided herein, including topical administration, administration by inhalation, and intravenous administration (e.g., continuous infusion).

In some embodiments, the multi-targeted TKI as provided herein can be administered in the manner of oral, buccal, inhalation spray, sublingual, rectal, transdermal, vaginal mucosa, transmucosal, local, nasal or enteral administration; parenteral administration, such as intramuscular injection, subcutaneous injection, intramedullary injection, as well as intrathecal or brain direct administration, in situ administration, subcutaneous, intraperitoneal, intravenous injection, intra-articular synovium, intrasternal, intrahepatic, intralesional, intracranial, intra-abdominal, nasal, intraocular injection or any other drug delivery manners.

In some embodiments, the pharmaceutical composition comprises multi-targeted TKI or a pharmaceutically acceptable salt thereof. In some embodiments, the pharmaceutical composition is in a solid dose form. In some embodiments, the solid dose form is capsules. In some embodiments, the solid dose form is dry-filled capsules. In some embodiments, the capsule comprises from about 10-500 mg of a multi-targeted TKI as provided herein.

In some embodiments, the multi-targeted TKI is administered at a dose from about 10 mg to 200 mg, 10 mg to 150 mg, 10 mg to 100 mg, 10 mg to 50 mg, or 20 mg to 50 mg. In some embodiments, the multi-targeted TKI is administered at a dose of about 10 mg, 20 mg, 30 mg, 40 mg, or 50 mg. In certain embodiments, olverembatinib is administered at a dose from about 10 mg to 200 mg, 10 mg to 150 mg, 10 mg to 100 mg, 10 mg to 50 mg, or 20 mg to 50 mg. In certain embodiments, olverembatinib is administered at a dose of about 10 mg, 20 mg, 30 mg, 40 mg, or 50 mg.

In some embodiments, the multi-targeted TKI as provided herein is administered once a day, once every other day, once a week, once every other week, once a month, or once every other month. In some embodiments, the multi-targeted TKI as provided herein is administered in one or more 7-days treatment cycles, 14-days treatment cycles, 28-days treatment cycles, 35-days treatment cycles, 42-days treatment cycles, two-months treatment cycles, three-months treatment cycles. In some embodiments, the treatment as provided herein comprises at least 1, 2, 3, 6, 14, 16 or 36 treatment cycles. In certain embodiments, olverembatinib is administered once a day, once every other day, once a week, once every other week, once a month, or once every other month. In certain embodiments, olverembatinib is administered in one or more 7-days treatment cycles, 14-days treatment cycles, 28-days treatment cycles, 35-days treatment cycles, 42-days treatment cycles, two-months treatment cycles, three-months treatment cycles. In certain embodiments, the treatment as provided herein comprises at least 1, 2, 3, 6, 14, 16 or 36 treatment cycles.

In preferred embodiments, the multi-targeted TKI as provided herein is orally administered once every other day in 28-days treatment cycles. In preferred embodiments, olverembatinib is orally administered once every other day in 28-days treatment cycles.

In some embodiments, following the treatment of the compound having formula (II), the subject experiences partial response or stable disease. In some embodiments, following cycles of 28-days treatment cycle of the compound having formula (II), the objective response rate (complete response and partial response) is about or above 20%, and the clinical benefit rate (complete response, partial response and stable disease) is about or above 90%.

In some embodiments, the method as provided herein further comprises further administering a therapeutically effective amount of one or more additional therapies. In some embodiments, the multi-targeted TKI is administered in combination with one or more additional therapies that treat the disease, disorder or condition as provided herein.

In some embodiments, the one or more additional therapies comprise a radiotherapy, a chemotherapy, a targeted cancer therapy, a therapy with a modulator of an immune checkpoint molecule, or a therapy with a BCL-2 inhibitor.

As used herein, the term “radiotherapy” refers to the treatment of cancers with ionizing radiation. The term “chemotherapy” refers to the treatment of cancers using specific chemical agents. The term “targeted cancer therapy” refers to the treatment of cancers with agents (chemical compounds or macromolecules) that selectively interact with a chosen biomolecule.

As used herein, an “immune checkpoint” or “immune checkpoint molecule” is a molecule in the immune system that modulates a signal. An immune checkpoint molecule can be a co-stimulatory checkpoint molecule, i.e., turn up a signal, or an inhibitory checkpoint molecule, i.e., turn down a signal. As used herein, a “modulator of an immune checkpoint molecule” is an agent capable of altering the activity of an immune checkpoint in a subject.

As used herein, a “Bcl-2 inhibitor” refers to an agent capable of downregulating the level and/activity of Bcl-2 in a subject.

### III. Methods for Treating Cancers with the Combination of Multi-Targeted KTI and BCL-2 Inhibitor

Described herein is a method of treating cancer in a subject, comprising administering to the subject, a) a therapeutically effective amount of a multi-targeted TKI; and b) a therapeutically effective amount of a Bcl-2 inhibitor.

### 3.1 Cancers

The combination as provided herein can be used for the treatment of cancers. In some embodiments, the cancer could be gastrointestinal cancer (e.g., GIST), cholangiocarcinoma, lung cancer, melanoma, breast cancer, colon cancer, ovarian cancer, prostate cancer, liver cancer (e.g., hepatocellular carcinoma), bladder cancer, pancreatic cancer, renal cancer, esophageal cancer, head and neck cancer, thyroid cancer, cutaneous squamous cell carcinoma, glioblastoma. neuroblastoma, urinary bladder cancer, hysterocarcinoma, melanoma, osteosarcoma, lymphoma (e.g., mantel cell lymphoma, diffuse large B cell lymphoma), leukemia (e.g., T-cell prolymphocytic leukemia, chronic lymphocytic leukemia, acute myeloid leukemia, acute lymphocytic leukemia (ALL)), multiple myeloma, uterine cancer, colorectal cancer, lung adenocarcinoma, uterine carcinosarcoma, lung squamous cell carcinoma, cervical cancer, esophagus cancer, sarcoma, chromophobe, renal cell carcinoma, clear cell RCC, papillary RCC, uveal melanoma, testicular germ cell tumor, low grade glioma (LGG), mesothelioma, pheochromocytoma, paraganglioma, thymoma, or adenoid cystic carcinoma (ACC). In preferred embodiments, the cancer is GIST.

In some embodiments, the subject as provided herein is determined or has been determined as comprising one or more mutations in KIT, PDGFR (e.g., PDGFRA and PDGFRB), SDH (e.g., SDHA, SDHB, SDHC and SDHD), NF1, BRAF, EGFR, HRAS, NRAS, KRAS, ZNF217, MEN1, PIK3CA, ARID1A, ARID1B, FGFR1, FGFR2, FGFR3, FGFR4, SDHAF2, FGFR1-HOOK3, FGFR1-TACC1, NTRK1 (e.g., NTRK1 fusions), NTRK2 (e.g., NTRK2 fusions), NTRK3 (e.g., NTRK3 fusions), KIT-PDGFRA, and PRKAR1B-BRAF, or any combination thereof (Lillian R. Klug et al., Nature Reviews Clinical Oncology. (2022) 19:328-341, Ji Hyun Lee et al., Cancers. 2022, 14 (11), 2659).

In some embodiments, the subject as provided herein is determined or has been determined as comprising one or more mutations in ADGRB3, AKT3, ALK, AMER1, APC, AR, ARAF, ASXL1, ATM, ATRX, BCL2, BCL9, BLM, BRIP1, BTG2, BTK, CBL, CCDC6/RET, CDK12, CDK8, CDKN2A, CDKN2B, CDKN2C, CHD2, CHEK2, CHIC2/KDR, COL1A1-PDGFB, CPEB3, CREB3L1, CREBBP, CSMD3, CTNNB1, DLL3, DPYD, DUSP2, EPAS1, EPCAM, ERBB2, ERBB3, ERBB4, ERCC4, FANCD2, FANCF, FAT1, FAT3, FH, FLT3, FOXA1, GRIN2A, GSK3B, HDAC1, IGF1R, IGF2, IGR/CDK8, IKBKE, IL7R, IQCJ-SCHIP1/PRKCI, JAK3, KMT2A, KMT2B, KMT2C, LRP1B, MAML3, MAP2K1, MAP3K1, MAX, MCL1, MDM4, MED12, MET, MGA, MLXIPL, MRE11, MSH2, MSH6, MTOR, MUC16, MUC4, MUTYH, MYD88, NAT1, NCOR2, NF2, NFKBIE, NKX2-1, NOTCH1, NOTCH2, NOTCH3, PAK3, PARP1, PDE11A, PGR, PIK3R1, PLK1, PMS2, POLD1, PRDM1, PRKN, PSIP1, PTCH1, PTEN, PTPN11, PTPN14, RAD51D, RB1, RBM10, RECQL4, RICTOR, RNF43, SBDS, SETBP1, SETD1B, SETD2, SF3B1, SGK1, SMAD4, SMARCB1, SMC1A, SMO, SRSF2, STAG2, STK11, TAP2, TCF7L1, TERC, TERT, TGFBR2, TNFAIP3, TNFRSF14, TP53, TRIM24, TSC1, TSC2, UGT1A1, USP44, USP8, VEGFA, WAS, WNK2, YAP1, ZEB1, and ZNF703, or any combination thereof.

In some embodiments, the subject as provided herein is determined or has been determined as not sensitive to at least one existing TKI. In some embodiments, if the subject does not obtain complete response (CR), partial response (PR), or stable disease (SD) within 6 months of the treatment of the at least one existing TKI, then the subject is defined as acquiring a primary resistance to the prior treatment of the existing TKI. In some embodiments, if the subject obtains initial response or stable disease following ≥6 months of the treatment of the at least one existing TKI and subsequent progression, then the subject is defined as acquiring an acquired resistance to the prior treatment of the existing TKI.

In some embodiments, the previously treated existing TKI could be imatinib (Novartis), ripretinib (ZAI Lab ( [symbol] )&Deciphera), avapritinib (CSTONE Pharmaceuticals ( [symbol] )&Blueprint), crenolanib (Pfizer), masitinib (AB SCIENCE), ponatinib (Takeda), dasatinib (BMS), nilotinib (Novartis), sunitinib (CPPI CV), regorafenib (BAYER), sorafenib (BAYER), anlotinib (CHIATAI TIANQING ( [symbol] [symbol] )), sulfatinib (HUTCHMED ( [symbol] ), pazopanib (GSK), rogaratinib (BAYER), infigratinib (BridgeBio), erdafitinib (JNJ), pemigatinib (Incyte), fisogatinib (Blueprint), famitinib (Hengrui ( [symbol] )), or any combinations thereof.

### 3.2 Multi-Targeted TKI and its Dosage Regimen

In some embodiments, the multi-targeted TKI is capable of inhibiting at least two tyrosine kinases selected from the group consisting of: KIT, PDGFR, SRC, VEGFR (e.g., any one of VEGFR1, VEGFR2, VEGFR3 or any combination thereof), FGFR (e.g., any one of FGFR1, FGFR2, FGFR3, FGFR4 or any combination thereof), or any combinations thereof.

In some embodiments, the multi-targeted TKI is capable of inhibiting at least two tyrosine kinases selected from the group consisting of: KIT, PDGFR, SRC, VEGFR2, FGFR1, or any combination thereof.

In some embodiments, the multi-targeted TKI comprises a heterocyclic alkyne benzene compound having the following formula (I):

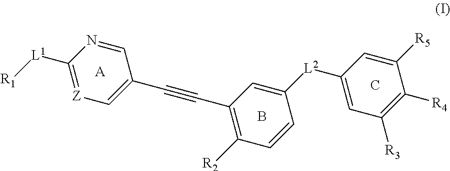

wherein Z is independently selected as CH or N; L¹ is independently selected from NH, —N═, CH; L² is independently selected as —CONH— or —NHCO—;
R₁ is independently selected from:
   1) H;
   2) C₁-C₆ alkyl;
   3) C₃-C₆ cycloalkyl;
   4) C₁-C₅ alkyl substituted by 1 or 2 hydroxy;
   5) phenyl;
   6) Groups which can fuse A ring through L¹ in Z atom site to form fused penta-heterocycles containing 1-3 N atoms and L¹ like

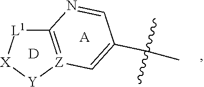

wherein, X, Y, Z are independently selected as N, CH; ring D is an aromatic heterocycle containing 1-3 N atoms;
R₂ is independently selected from
   1) H;
   2) Halogen;
   3) C₁-C₅ alkyl;
   4) C₃-C₆ cycloalkyl;
   5) C₁-C₅ alkyl containing F;
R₃ is independently selected from
   1) H;
   2) Halogen;
   3) C₁-C₄ alkyl;
   4) C₃-C₆ cycloalkyl;
   5) C₁-C₄ alkyl containing F;
when R₅ is H, R₄ is independently selected from
   1) H;
   2) (CH₂)ₙNR₆R₇;
   3) (CH₂)ₙ-Het¹;
or, when R₄ is H, R₅ is independently selected from
   1) H;
   2) Het²;
   n is independently selected from 0 or 1; Het¹ is defined as nonaromatic heterocycle containing 1-3 N atoms; Het² is defined as aromatic five-member heterocycle containing hetero atoms like N, O, and/or S; alkyl, cycloalkyl or NR₆R₇ may be incorporated into any C or N position in the nonaromatic heterocycle and aromatic heterocycle which can be substituted;
R₆ or R₇ is independently selected from:
   1) H;
   2) C₁-C₃ alkyl;
   3) C₁-C₃ alkyl containing F;
   4) C₃-C₆ cycloalkyl;
   or R₆ and R₇ can further form penta-, hexa-, hepta- or octatomic ring structure through C, O, N, S atoms;
or a pharmaceutically acceptable salt or a stereoisomer or a prodrug thereof.

In some embodiments, the multi-targeted TKI is a compound having the following formula (II):

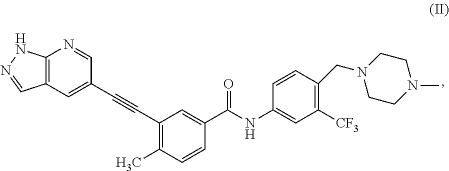 [SMILES: Cc1ccc(C(=O)Nc2ccc(CN3CCN(C)CC3)c(C(F)(F)F)c2)cc1C#Cc1cnc2[nH]ncc2c1]

or a pharmaceutically acceptable salt or a stereoisomer or a prodrug thereof.

In some embodiments, the multi-targeted TKI as provided herein is a compound selected from the group consisting of: olverembatinib, ripretinib, avapritinib, crenolanib, masitinib, ponatinib, dasatinib, nilotinib, sunitinib, regorafenib, sorafenib, anlotinib, sulfatinib, infigratinib, erdafitinib, pemigatinib, rogaratinib, and linsitinib, or a pharmaceutically acceptable salt or a stereoisomer or a prodrug of any of the foregoing.

In some embodiments, the multi-targeted TKI as provided herein is administered at a dose of about 1 mg/kg, 5 mg/kg, 10 mg/kg, 15 mg/kg, 20 mg/kg, 25 mg/kg, 50 mg/kg, 75 mg/kg, 100 mg/kg, 150 mg/kg, or 200 mg/kg. In certain embodiments, olverembatinib is administered at a dose of about 1 mg/kg, 5 mg/kg, 10 mg/kg, 15 mg/kg, 20 mg/kg, 25 mg/kg, 50 mg/kg, 75 mg/kg, 100 mg/kg, 150 mg/kg, or 200 mg/kg.

In some embodiments, the multi-targeted TKI as provided herein is administered once a day, once every other day, once a week, once every other week, once a month, or once every other month. In certain embodiments, olverembatinib is administered once a day, once every other day, once a week, once every other week, once a month, or once every other month.

In some embodiments, the multi-targeted TKI as provided herein is administered for at least 1 week, 2 weeks, 3 weeks, 4 weeks, 2 months, 3 months, or 4 months. In certain embodiments, olverembatinib is administered for at least 1 week, 2 weeks, 3 weeks, 4 weeks, 2 months, 3 months, or 4 months.

In some embodiments, the multi-targeted TKI as provided herein (e.g., olverembatinib) is administered by oral route or parental route (e.g., intravenous, intramuscular, subcutaneous, intramedullary, intrathecal, intraventricular, intraperitoneal, intranasal, or intraocular injection).

In some embodiments, the multi-targeted TKI as provided herein is administered once every other day for 3 weeks at a dose of about 15 mg/kg. In certain embodiments, olverembatinib is administered once every other day for 3 weeks at a dose of about 15 mg/kg.

### 3.3 Bcl-2 Inhibitor and its Dosage Regimen

Bcl-2 family proteins are important regulators of apoptosis. The family of proteins includes anti-apoptotic proteins such as Bcl-2, Bcl-xL and Mcl-1; and pro-apoptotic molecules, including Bid, Bim, Bad, Bak and Bax. Although normal cells have low expression levels of anti-apoptotic Bcl-2 and Bcl-xL proteins, these proteins are found to be highly overexpressed in many different types of human tumors and are implied in tumor development, progression and resistance to drugs. As used herein, a “Bcl-2 inhibitor” refers to an agent capable of downregulating the level and/activity of Bcl-2 in a subject.

In some embodiments, the Bcl-2 inhibitor as provided herein is a compound having the following formula (III)

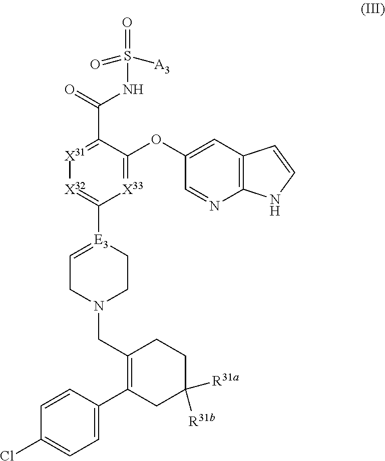

or a pharmaceutically acceptable salt or a stereoisomer or a prodrug thereof, wherein:
A₃ is selected from

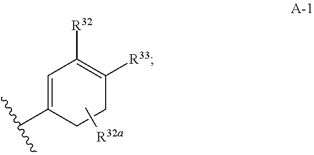

E₃ is a nitrogen atom and [symbol] is a single bond;
X³¹, X³², and X³³ are each independently selected from the group consisting of —CR³⁸═ and —N═;
R^{31a} and R^{31b} taken together with the carbon atom to which they are attached form a 3-, 4-, or 5-membered cycloalkyl;
R³² is selected from the group consisting of —NO₂, —SO₂CH₃, and —SO₂CF₃;
R^{32a} is selected from the group consisting of hydrogen and halogen;
R³³ is selected from —N(R^{34a})(R^{34b});
R^{34a} is selected from the group consisting of optionally substituted C₁₋₆ alkyl, optionally substituted C₃₋₆ cycloalkyl, heterocyclo, heteroalkyl, (cycloalkyl)alkyl, and (heterocyclo)alkyl;
R^{34b} is selected from the group consisting of hydrogen and C₁₋₄ alkyl;
R³⁸ is selected from the group consisting of hydrogen and halogen.

In some embodiments, the Bcl-2 inhibitor is selected from the group consisting of:

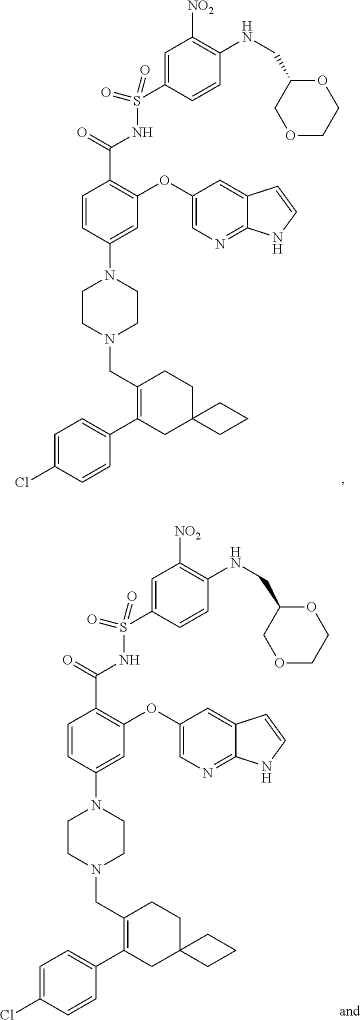 [SMILES: O=C(NS(=O)(=O)c1ccc(NC[C@H]2COCCO2)c([N+](=O)[O-])c1)c1ccc(N2CCN(CC3=C(c4ccc(Cl)cc4)CC4(CCC4)CC3)CC2)cc1Oc1cnc2[nH]ccc2c1 ; O=C(NS(=O)(=O)c1ccc(NC[C@@H]2COCCO2)c([N+](=O)[O-])c1)c1ccc(N2CCN(CC3=C(c4ccc(Cl)cc4)CC4(CCC4)CC3)CC2)cc1Oc1cnc2[nH]ccc2c1]

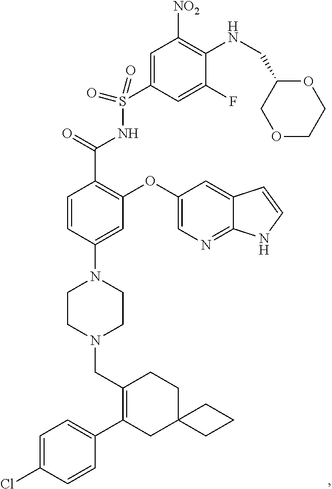 [SMILES: O=C(NS(=O)(=O)c1cc(F)c(NC[C@H]2COCCO2)c([N+](=O)[O-])c1)c1ccc(N2CCN(CC3=C(c4ccc(Cl)cc4)CC4(CCC4)CC3)CC2)cc1Oc1cnc2[nH]ccc2c1]

or a pharmaceutically acceptable salt or a stereoisomer or a prodrug thereof.

In preferred embodiments, the Bcl-2 inhibitor is a compound having the following formula (IV) (also known as lisaftoclax, APG-2575)

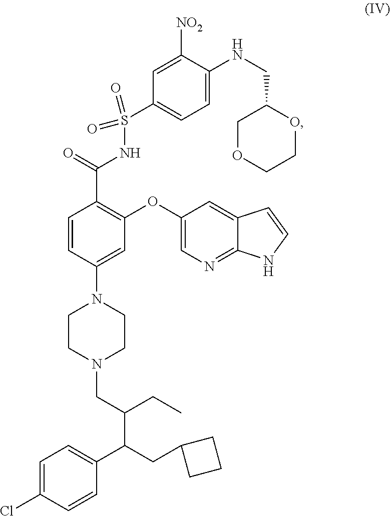 [SMILES: CCC(CN1CCN(c2ccc(C(=O)NS(=O)(=O)c3ccc(NC[C@H]4COCCO4)c([N+](=O)[O-])c3)c(Oc3cnc4[nH]ccc4c3)c2)CC1)C(CC1CCC1)c1ccc(Cl)cc1]

or a pharmaceutically acceptable salt or a stereoisomer or a prodrug thereof.

More multi-targeted TKI that can be used in the present application are further disclosed in WO2018027097A1, which is incorporated herein by reference.

One skilled in the art would be able, by routine experimentation, to determine what an effective, non-toxic amount of the Bcl-2 inhibitor as provided herein would be for the purpose of treating cancers as provided herein. For example, a therapeutically active amount of Bcl-2 inhibitor may vary according to factors such as the disease stage (e.g., stage I versus stage IV), age, sex, medical complications (e.g., immunosuppressed conditions or diseases) and weight of the subject, and the ability of the Bcl-2 inhibitor to elicit a desired response in the subject. In certain embodiments, a therapeutically active amount is a safe amount of the Bcl-2 inhibitor which is sufficient to yield a desired therapeutic response without undue adverse side effects (such as toxicity, irritation, or allergic response) commensurate with a reasonable benefit/risk ratio when used in the manner of this disclosure.

The dosage regimen may be adjusted to provide the optimum therapeutic response. For example, several divided doses may be administered daily or administered by continuous dosing or the dose may be proportionally reduced as indicated by the exigencies of the therapeutic situation. In certain embodiments, the Bcl-2 inhibitor as provided herein is administered in an amount that would be therapeutically effective if delivered alone or in combination, i.e., multi-targeted TKI is administered and/or acts as a therapeutic treatment of the desired cancers, and not predominantly as an agent to ameliorate side effects of other treatments.

In some embodiments, the Bel-2 inhibitor as provided herein is administered in an amount that would be effective in the treatment of the cancers. The dosages provided below may be used for any mode of administration of the multi-targeted TKI as provided herein, including topical administration, administration by inhalation, and intravenous administration (e.g., continuous infusion).

In some embodiments, the Bcl-2 inhibitor as provided herein can be administered in the manner of oral, buccal, inhalation spray, sublingual, rectal, transdermal, vaginal mucosa, transmucosal, local, nasal or enteral administration; parenteral administration, such as intramuscular injection, subcutaneous injection, intramedullary injection, as well as intrathecal or brain direct administration, in situ administration, subcutaneous, intraperitoneal, intravenous injection, intra-articular synovium, intrasternal, intrahepatic, intralesional, intracranial, intra-abdominal, nasal, intraocular injection or any other drug delivery manners.

In some embodiments, the pharmaceutical composition comprises Bcl-2 inhibitor or a pharmaceutically acceptable salt thereof. In some embodiments, the pharmaceutical composition is in a solid dose form. In some embodiments, the solid dose form is capsules. In some embodiments, the solid dose form is dry-filled capsules. In some embodiments, the capsule comprises from about 10-500 mg of a Bcl-2 inhibitor as provided herein.

In some embodiments, the Bel-2 inhibitor as provided herein is administered at a dose of about 1 mg/kg, 5 mg/kg, 10 mg/kg, 15 mg/kg, 20 mg/kg, 25 mg/kg, 50 mg/kg, 75 mg/kg, 100 mg/kg, 150 mg/kg, or 200 mg/kg. In certain embodiments, lisaftoclax is administered at a dose of about 1 mg/kg, 5 mg/kg, 10 mg/kg, 15 mg/kg, 20 mg/kg, 25 mg/kg, 50 mg/kg, 75 mg/kg, 100 mg/kg, 150 mg/kg, or 200 mg/kg.

In some embodiments, the Bel-2 inhibitor as provided herein is administered twice a day, once a day, once every other day, once a week, once every other week, once a month, or once every other month. In some embodiments, the Bcl-2 inhibitor as provided herein is administered for at least 1 week, 2 weeks, 3 weeks, 4 weeks, 2 months, 3 months or 4 months. In certain embodiments, lisaftoclax is administered twice a day, once a day, once every other day, once a week, once every other week, once a month, or once every other month. In certain embodiments, lisaftoclax is administered for at least 1 week, 2 weeks, 3 weeks, 4 weeks, 2 months, 3 months or 4 months.

In preferred embodiments, the Bcl-2 inhibitor as provided herein is administered once a day for 3 weeks at a dose of about 50 mg/kg. In preferred embodiments, lisaftoclax is administered once a day for 3 weeks at a dose of about 50 mg/kg.

### 3.4 Combination Therapy

Provided herein is a method for treating cancers with a combination of a multi-targeted KTI and a Bcl-2 inhibitor, wherein the multi-targeted KTI and the Bel-2 inhibitor are respectively as defined above. The cancer is also as defined above.

In some embodiments, the multi-targeted TKI and the Bel-2 inhibitor are each in a separate preparation, or the multi-targeted TKI and the Bel-2 inhibitor are in an individual preparation.

In some embodiments, the multi-targeted TKI and the Bcl-2 inhibitor are administered concurrently, or the multi-targeted TKI and the Bcl-2 inhibitor are administered sequentially.

In some embodiments, the multi-targeted TKI and the Bcl-2 inhibitor may be administered sequentially at a time interval of about 1 minute, about 5 minutes, about 10 minutes, about 15 minutes, about 30 minutes, about 45 minutes, about 1 hour, about 2 hours, about 4 hours, about 6 hours, about 12 hours, about 24 hours, about 48 hours, about 72 hours, about 96 hours, about 1 week, about 2 weeks, about 3 weeks, about 4 weeks, about 5 weeks, about 6 weeks, about 8 weeks, or about 12 weeks.

In some embodiments, the combination product of the invention comprising the multi-targeted TKI and the Bcl-2 inhibitor can be in the form of a pharmaceutical composition, where the multi-targeted TKI and the Bcl-2 inhibitor are each in a separate dosage unit form, or the multi-targeted TKI and the Bcl-2 inhibitor are in the form of a single dosage unit.

In some embodiments, the combination product can be administered in the following manner: oral, buccal, inhalation spray, sublingual, rectal, transdermal, vaginal mucosa, transmucosal, local, nasal or enteral administration; parenteral administration, such as intramuscular injection, subcutaneous injection, intramedullary injection, as well as intrathecal or brain direct administration, in situ administration, subcutaneous, intraperitoneal, intravenous injection, intra-articular synovium, intrasternal, intrahepatic, intralesional, intracranial, intra-abdominal, nasal, intraocular injection or any other drug delivery manners.

In some embodiments, the multi-targeted TKI comprised in the combination therapy is administered at a dose of about 1 mg/kg, 5 mg/kg, 10 mg/kg, 15 mg/kg, 20 mg/kg, 25 mg/kg, 50 mg/kg, 75 mg/kg, 100 mg/kg, 150 mg/kg, or 200 mg/kg; and the Bcl-2 inhibitor comprised in the combination therapy is administered at a dose of about 1 mg/kg, 5 mg/kg, 10 mg/kg, 15 mg/kg, 20 mg/kg, 25 mg/kg, 50 mg/kg, 75 mg/kg, 100 mg/kg, 150 mg/kg, or 200 mg/kg. In certain embodiments, the olverembatinib comprised in the combination therapy is administered at a dose of about 1 mg/kg, 5 mg/kg, 10 mg/kg, 15 mg/kg, 20 mg/kg, 25 mg/kg, 50 mg/kg, 75 mg/kg, 100 mg/kg, 150 mg/kg, or 200 mg/kg; and the lisaftoclax comprised in the combination therapy is administered at a dose of about 1 mg/kg, 5 mg/kg, 10 mg/kg, 15 mg/kg, 20 mg/kg, 25 mg/kg, 50 mg/kg, 75 mg/kg, 100 mg/kg, 150 mg/kg, or 200 mg/kg.

In some embodiments, the multi-targeted TKI comprised in the combination therapy and the Bcl-2 inhibitor comprised in the combination therapy are administered once a day, once every other day, once a week, once every other week, once a month, or once every other month, separately or together. In certain embodiments, the olverembatinib comprised in the combination therapy and the lisaftoclax comprised in the combination therapy are administered once a day, once every other day, once a week, once every other week, once a month, or once every other month, separately or together.

In some embodiments, the combination therapy as provided herein comprising the multi-targeted TKI and the Bel-2 inhibitor is administered for at least 1 week, 2 weeks, 3 weeks, 4 weeks, 2 months, 3 months, or 4 months. In certain embodiments, the combination therapy as provided herein comprising olverembatinib and lisaftoclax is administered for at least 1 week, 2 weeks, 3 weeks, 4 weeks, 2 months, 3 months, or 4 months.

In preferred embodiments, the multi-targeted TKI as provided herein is administered once every other day for 3 weeks at a dose of about 15 mg/kg, and the Bcl-2 inhibitor as provided herein is administered once a day for 3 weeks at a dose of about 50 mg/kg. In preferred embodiments, olverembatinib is administered once every other day for 3 weeks at a dose of about 15 mg/kg, and lisaftoclax is administered once a day for 3 weeks at a dose of about 50 mg/kg.

The combination therapy of the multi-targeted TKI as provided herein (e.g., olverembatinib) and the Bcl-2 inhibitor as provided herein (e.g., lisaftoclax) may produces supplemental, additive or synergistic effects as compared to a monotherapy (i.e., either the multi-targeted TKI as provided herein alone, or the Bel-2 inhibitor as provided herein alone). A combination therapy that produces supplemental effects means that the overall clinical benefits produced by the combination therapy is greater than those of a monotherapy. A combination therapy that produces additive effects means that the clinical benefits of the agents in combination reflect a sum of the monotherapies. A combination therapy that produces synergistic effects means that the overall clinical benefits achieved in combination are greater than the additive effects of each agent. Moreover, in some embodiments, due to the additive or synergistic effects of a combination therapy, less frequent dosing of one or more of the therapies and/or lower doses of one or more therapies, as compared to administration of the monotherapy alone, may be used. In further embodiments, due to lower dosages and/or less frequent dosing of one or more of the therapies of a combination therapy may result in less toxicity due to fewer side effects from one or more of the therapies. Accordingly, in some embodiments, a pharmaceutically effective amount of an agent used as a component of a combination therapy to treat a disease in a subject is less than a pharmaceutically amount of the same agent used as a monotherapy.

In some embodiments, following the treatment of the compound having formula (II) in combination with the compound having formula (IV), the subject has tumor growth inhibition (TGI) rates of at least 75%.

In some embodiments, the multi-targeted TKI in combination with the Bcl-2 inhibitor are capable of inhibiting proliferation of imatinib-resistant GIST cells. In some embodiments, the multi-targeted TKI in combination with the Bcl-2 inhibitor are capable of inhibiting proliferation of imatinib-resistant GIST cells in an additive manner or a synergistic manner. The imatinib-resistant GIST cell used herein could be GIST430, GIST48, GIST48B, or any combination thereof.

The following examples are provided to better illustrate the claimed invention and are not to be interpreted as limiting the scope of the invention. All specific compositions, materials, and methods described below, in whole or in part, fall within the scope of the present invention. These specific compositions, materials, and methods are not intended to limit the invention, but merely to illustrate specific embodiments falling within the scope of the invention. One skilled in the art may develop equivalent compositions, materials, and methods without the exercise of inventive capacity and without departing from the scope of the invention. It will be understood that many variations can be made in the procedures herein described while still remaining within the bounds of the present invention. It is the intention of the inventors that such variations are included within the scope of the invention.

### EXAMPLES

While we have described a number of embodiments of this invention, it is apparent that our basic examples may be altered to provide other embodiments that utilize the compounds and methods of this disclosure. Therefore, it will be appreciated that the scope of this disclosure is to be defined by the appended claims rather than by the specific embodiments that have been represented by way of example.

The contents of all references (including literature references, issued patents, published patent applications, and co-pending patent applications) cited throughout this application are hereby expressly incorporated herein in their entireties by reference. Unless otherwise defined, all technical and scientific terms used herein are accorded the meaning commonly known to one with ordinary skill in the art.

### Example 1. Olverembatinib in Advanced SDH-Deficient GISTs: A Multicenter, Open-Label, Phase 1 Trial

### 1. Methods

### Study Design and Patient Population

HQP1351-SJ-0003 is an ongoing open-label, dose-escalation/dose-expansion phase 1 study being conducted at six sites across China to evaluate the safety and efficacy of olverembatinib in patients with advanced and/or metastatic GIST or other solid tumors without appropriate treatment (ClinicalTrials.gov identifier: NCT03594422). The dose escalation aspect of the study enrolled patients with advanced and/or metastatic GIST or other solid tumors that were refractory or had relapsed after treatment with at least one TKI. In this group, oral olverembatinib was administered QOD at starting doses of 20, 30, 40, or 50 mg, in continuous 28-day cycles until the MTD was determined, with the aim of advancing to the dose-expansion part of the study to determine RP2D.

Eligible patients were adults (≥18 years) who had advanced and/or metastatic GISTs or other solid tumors confirmed by histology and/or cytology. The definition of SDH-deficient GIST was based on pathological phenotype with negative immunohistochemical staining for SDHB. Also required for study enrollment were Eastern Cooperative Oncology Group performance status (ECOG PS)≤2; and adequate renal, hepatic, cardiac, and bone marrow function. A full list of inclusion and exclusion criteria is in the protocol. Enrollment trends, ongoing review of study data, and evolving knowledge regarding the GIST treatment paradigm informed the study sponsor's medical director's and investigators' decision to analyze the SDH-deficient GIST patient subgroup separately.

Based on encouraging initial activity and favorable tolerability (Qiu, H., et al., J. Clin. Oncol. 40, 11513 (2022)), additional patients with SDH-deficient GIST were enrolled to further explore efficacy, and patients with TKI-naïve SDH-deficient GIST and adolescent patients (aged 12-18 years old) with SDH-deficient GIST were permitted. After data from the dose-escalation part of the study were reviewed during a meeting of the principal investigators and a postmarket dose was recommended to treat CML, all adult patients received 40 mg QOD (determined RP2D dose), and adolescent patients received 20 to 40 mg QOD based on body weight. If body weight was at least 40 kg, patients received 40 mg QOD; if within 30-40 kg, 30 mg QOD; and, if within 20-30 kg, 20 mg QOD. Patients with SDH-deficient malignant paraganglioma who had failed at least one treatment and were without appropriate treatment (as judged by investigators) were also enrolled. Treatment could continue until precluded by toxicity, noncompliance, withdrawal of consent, physician decision, progressive disease, death, or closure of the study. All protocol modifications, including those increasing the number of patients and cohorts in the dose-expansion aspect, are established in the trial protocol version dated May 27, 2022.

Tumor mutation status was determined by local testing and centrally confirmed using DNA from archival or new tumor biopsy samples. The study was performed in accordance with ethical principles of the Declaration of Helsinki and was consistent with the International Conference on Harmonisation Good Clinical Practice. The institutional review board or independent ethics committee of each site approved the study. All patients provided written informed consent.

### Study Assessments

Physical examination and adverse events (AEs) were evaluated at each follow-up visit (day −28~day −1 after signed informed consent form); on days 1, 8, 15, and 22 during cycles 1 and 2; and on days 1 and 15 in cycle 3 and all subsequent cycles, up to 30 days after the final dose. All AEs were graded according to the US National Cancer Institute (NCI) Common Terminology Criteria for Adverse Events (CTCAE; version 4.03). Clinical laboratory investigations for routine blood examinations and serum chemistry parameters were performed during every cycle (on day 1); cycles 1 and 2 (on days 8, 15, and 22); and 30 days after the last dose of treatment. Urinalysis, coagulation, cardiac troponin I/T, lipase, amylase, and electrocardiograms were done at baseline; during cycles 1 and 2 (on days 1 and 15); cycle 3, 5, 7, and 9; every 3 cycles after cycle 9 (on day 1); and 14 days after the last dose of treatment. All patients underwent tumor imaging for response assessment via computed tomography or magnetic resonance imaging at screening and every two cycles (8 weeks) until progression or discontinuation. Target and nontarget lesions were assessed per Response Evaluation Criteria in Solid Tumors version 1.1 (RECIST v1.1).

Serial blood samples were collected to measure olverembatinib plasma concentrations. For adult patients, plasma samples were collected before, and 0.5, 1, 2, 4, 6, 8, 12, 24, and 48 hours after, dosing on days 1 and 27 of cycle 1 and before dosing on days 15, 17, and 19. For adolescent patients, plasma samples were collected before, and 1, 2, 4, 6, 8, 24, and 48 hours after, dosing on days 1 and 27 of cycle 1. Noncompartmental pharmacokinetic (PK) analysis was conducted using Phoenix WinNonlin software (Version 6.4). PK parameters were derived for olverembatinib, including area under the plasma concentration-time curve (AUC), maximum observed plasma concentration (Cₘₐₓ), time of maximum observed plasma concentration (tₘₐₓ), apparent terminal elimination half-life (t_{1/2}), apparent total oral clearance (CL/F), and apparent volume of distribution during the terminal elimination phase (Vz/F).

### Study Outcomes

Primary end points were determination of MTD/RP2D and the safety profile of olverembatinib. Secondary end points included PK and pharmacodynamic properties, CBR, overall response rate (ORR), PFS, and overall survival (OS) per RECIST v1.1.

CBR was defined as the proportion of patients with a confirmed CR or PR of any duration or SD for at least 16 weeks from the start of treatment (CBR=CR+PR+SD≥16 weeks). ORR was defined as the proportion of patients with a confirmed CR or PR of any duration (ORR=CR+PR). PFS was defined as the time from the start of treatment to the date of first documented disease progression or death due to any cause, whichever occurred first. OS was defined as the time from start of treatment until the date of death.

### Statistical Analysis

The data cutoff for this analysis was Dec. 27, 2023. The safety population included all patients who received at least one dose of olverembatinib. The SDH-deficient GIST population (prespecified subgroup) included patients in the safety population. MTD was defined as the highest dose level with no more than one dose-limiting toxicity in six patients.

We evaluated antitumor activity in patients from the SDH-deficient GIST population who had at least one postbaseline disease assessment. We used Kaplan-Meier methods to estimate PFS, and OS; corresponding 95% CIs are provided. Efficacy results for patients with SDH-deficient GIST are reported separately. All statistical analyses were performed with SAS (version 9.3 or higher; SAS Institute, Cary, NC).

### 2. Results

### Patients

From Jul. 11, 2018, through Dec. 27, 2023, 66 patients were enrolled in the safety population. The study population included 33 patients with non-SDH-deficient GIST, 26 SDH-deficient GIST, 6 with paraganglioma, and 1 with pancreatic cancer. Baseline characteristics of patients in the non-SDH-deficient and SDH-deficient groups are presented in Table 2. In the non-SDH-deficient GIST group (n=33), the median age was 55 (range 33-72) years; 26 (78.8%) patients were men; and the primary tumor sites were the stomach in 6 (18.2%) patients, small intestine in 18 (54.5%), and large intestine in 2 (6.1%). Most patients had received previous TKI treatment, with 15 (45.5%) and 13 (39.4%) having received at least 2 or 3 TKIs, respectively. In the SDH-deficient GIST group (n=26), the median age was 30 (range, 13-56) years; 7 (26.9%) patients were men; and the primary tumor site was the stomach in 24 patients (92.3%). Most patients had been treated with TKIs, including 5 (19.2%), 9 (34.6%), and 2 (7.7%) receiving at least 2, 3, and 4 or more TKIs, respectively.

TABLE 2
Baseline characteristics of GIST patients

| | Non-SDH- | | SDH- | | | |
|---|---|---|---|---|---|---|
| | deficient | | deficient | | | |
| | GIST | | GIST | | Total | |
| | (N = 33) | | (N = 26) | | (N = 59) | |
| Age (range), yr | 55.0 | (33-72) | 30.0 | (13-56) | 45.0 | (13-72) |
| Sex, n (%) | | | | | | |
| Male | 26 | (78.8%) | 7 | (26.9%) | 33 | (55.9%) |
| Female | 7 | (21.2%) | 19 | (73.1%) | 26 | (44.1%) |
| Primary tumor | | | | | | |
| site, n (%) | | | | | | |
| Stomach | 6 | (18.2%) | 24 | (92.3%) | 30 | (50.8%) |
| Small intestine* | 18 | (54.5%) | 0 | | 18 | (30.5%) |
| Large intestine^{†} | 2 | (6.1%) | 0 | | 2 | (3.4%) |
| Other | 7 | (21.2%) | 2^{‡} | (7.7%) | 9 | (15.3%) |
| ECOG; n (%) | | | | | | |
| 0 | 12 | (36.4%) | 11 | (42.3%) | | |
| 1 | 18 | (54.5%) | 13 | (50.0%) | | |
| 2 | 3 | (9.1%) | 0 | | | |
| Missing | 0 | | 2 | (7.7%) | | |
| No. of previous | | | | | | |
| TKIs; n (%) | | | | | | |
| 0 | 0 | | 1 | (3.8%) | | |
| 1 | 5 | (15.2%) | 9 | (34.6%) | | |
| 2 | 15 | (45.5%) | 5 | (19.2%) | | |
| 3 | 13 | (39.4%) | 9 | (34.6%) | | |

| | | | |
|---|---|---|---|
| ≥4 | 0 | 2 | (7.7%) |

Gene mutations

| | | | |
|---|---|---|---|
| KIT | 27/33 | SDHA | 1/26 |
| | (81.8%) | | (3.8%) |
| PDGFRA | 2/33 | SDHB | 12/26 |
| | (6.1%) | | (46.2%) |
| Wild-type | 2/33 | SDHC | 1/26 |
| | (6.1%) | | (3.8%) |

| | | |
|---|---|---|
| 1 SDH competent, | No SDH | 12/26 |
| NF1 positive | mutation | (46.2%) |
| 1 unknown (tissue | | |
| not available) | | |

Data are median (range) or n (%). Proportions might not add to 100% as a result of rounding. ECOG = Eastern Cooperative Oncology Group.
*Small intestine, duodenum, jejunum, or ileum.
^{†}Colon and rectum.
^{‡}Unknown: one patient's initially assessed tumor was multiple liver metastases; another was a large abdominal mass.

Of 33 patients with non-SDH-deficient GIST, 27 (81.8%) had KIT mutations, 2 (6.1%) PDGFRA mutations, and 2 (6.1%) WT GIST. In this last subgroup, one patient was SDH-competent and neurofibromatosis 1 (NF1)-positive, and the other patient's SDH status was unknown because tissue was not available for immunohistochemical staining. Of 26 patients with SDH-deficient GIST, germline SDHB was the most common SDH mutation (12 patients, 46.2%), 1 patient each had germline SDHA and SDHC mutations, and the remaining 12 (46.2%) did not have detectable SDH mutations.

Distributions of different GIST subtypes and dosages of olverembatinib are summarized in Table 3. Most patients received olverembatinib 40 mg on alternate days (QOD), which was selected as the RP2D based on data from the dose-escalation part of this study and several studies of olverembatinib in CML. This indication and the usage of 40 mg QOD were approved for marketing by the Chinese National Medical Products Administration.

TABLE 3
Distribution of different diseases/GIST subtypes and olverembatinib dosages

| | 20 mg | 30 mg | 40 mg | 50 mg | |
|---|---|---|---|---|---|
| | QOD | QOD | QOD | QOD | Total |
| | (N = 3) | (N = 15) | (N = 32) | (N = 16) | (N = 66) |
| KIT-mutated GIST | 2 | 8 | 10 | 9 | 29 |
| PDGFRA-mutated GIST | 1 | | 1 | | 2 |
| Wild-type GIST | | 1 | | 1 | 2 |
| SDH-deficient GIST | 0 | 6 (1 | 14 (1 | 6 | 26 |
| | | adolescent) | adolescent) | | |
| SDH-deficient | — | — | 6 | — | 6 |
| paraganglioma | | | | | |
| Pancreatic cancer | — | — | 1 | — | 1 |

### Safety

At the data cutoff, the median duration of follow-up was 14.5 (range, 0.9-57.5) months. Based on investigator judgment, all 66 subjects (100.0%) experienced treatment-emergent adverse events (TEAEs) and most were grade 1 or 2. TEAEs (≥20%) included increased aspartate aminotransferase (AST) in 31 patients (47%), increased alanine aminotransferase (ALT) in 25 (37.9%), increased leukocyte count in 32 (48.5%), increased neutrophil count in 25 (37.9%), anemia in 31 (47.0%), pyrexia in 28 (42.4%), constipation in 27 (40.9%), and abdominal pain in 15 (22.7%) (Table 4). Sixty-two patients (93.9%) experienced treatment-related adverse events (TRAEs), most of which were grade 1 or 2 (Table 5). Nine patients (13.6%) experienced grade ≥3 TRAEs, and 5 (7.6%) treatment-related serious adverse events were reported (Table 5). No dose-limiting toxicities were reported, and the maximum tolerated dose (MTD) was not reached. A total of 21 (31.8%) of 66 patients required treatment interruption, and the most common reasons were COVID-19 (9 patients), pyrexia (4 patients), and 2 each for abdominal pain and increased AST. TRAEs led to dose reductions in two patients, because of either increased AST or patient choice in one each.

TABLE 4
Treatment-emergent adverse events (AEs) in all patients

| Treatment-emergent AEs | Any | Grades |
|---|---|---|
| reported in ≥20% of | grade | 3-5 |
| patients (N = 66) | N (%) | N (%) |
| Increased aspartate | 31 (47.0) | 2 (3.0) |
| aminotransferase | | |
| Anemia | 31 (47.0) | 4 (6.1) |
| Increased leukocyte count | 32 (48.5) | 0 |
| Hyperuricemia | 31 (47.0) | 1 (1.5) |
| Pyrexia | 28 (42.4) | 0 |
| Increased alanine | 25 (37.9) | 2 (3.0) |
| aminotransferase | | |
| Constipation | 27 (40.9) | 0 |
| Increased neutrophil count | 25 (37.9) | 0 |
| Fatigue | 20 (30.3) | 1 (1.5) |
| Abdominal pain | 15 (22.7) | 1 (1.5) |
| Serious treatment-emergent AEs | | |
| reported in ≥2 patients (N = 66) | N (%) | |
| Intestinal obstruction | 5 (7.6) | |
| Anemia | 2 (3.0) | |

TABLE 5
Treatment-related adverse events (AEs) of all patients

| | | |
|---|---|---|
| Treatment-related AEs | Any | Grades |
| reported in ≥10% of | grade | 3-5, |
| patients (N = 66) | N (%) | N (%) |

| Increased WBC count | 27 | (40.9) | 0 | |
|---|---|---|---|---|
| Increased aspartate | 23 | (34.8) | 1 | (1.5%) |
| aminotransferase | | | | |
| Increased neutrophil count | 22 | (33.3) | 0 | |
| Constipation | 23 | (34.8) | 0 | |
| Increased alanine | 20 | (30.3) | 1 | (1.5) |
| aminotransferase | | | | |
| Fatigue | 18 | (27.3) | 1 | (1.5) |
| Melanocytic nevi | 15 | (22.7) | 0 | |
| Hypoalbuminemia | 10 | (15.2) | 0 | |
| Hyperuricemia | 12 | (18.2) | 0 | |
| Increased CRP | 11 | (16.7) | 0 | |
| Increased creatinine | 9 | (13.6) | 0 | |
| Anemia | 9 | (13.6) | 1 | (1.5) |
| Increased platelet count | 7 | (10.6) | 0 | |
| Decreased neutrophil count | 3 | (4.5) | 2 | (3.0) |
| Abdominal pain | 3 | (4.5) | 1 | (1.5) |
| Intestinal obstruction | 2 | (3.0) | 1 | (1.5%) |
| Hypertension | 3 | (4.5) | 1 | (1.5) |
| Leukemoid reaction | 1 | (1.5) | 1 | (1.5) |
| Coronary sclerosis | 1 | (1.5) | 1 | (1.5) |

| | |
|---|---|
| Treatment-related serious AEs | N (%) |

| | | |
|---|---|---|
| Anemia | 2 | (3.0) |
| Intestinal obstruction | 1 | (1.5) |
| Leukemoid reaction | 1 | (1.5) |
| Coronary sclerosis | 1 | (1.5) |

CRP, C-reactive protein; WBC, white blood cell.

### Efficacy

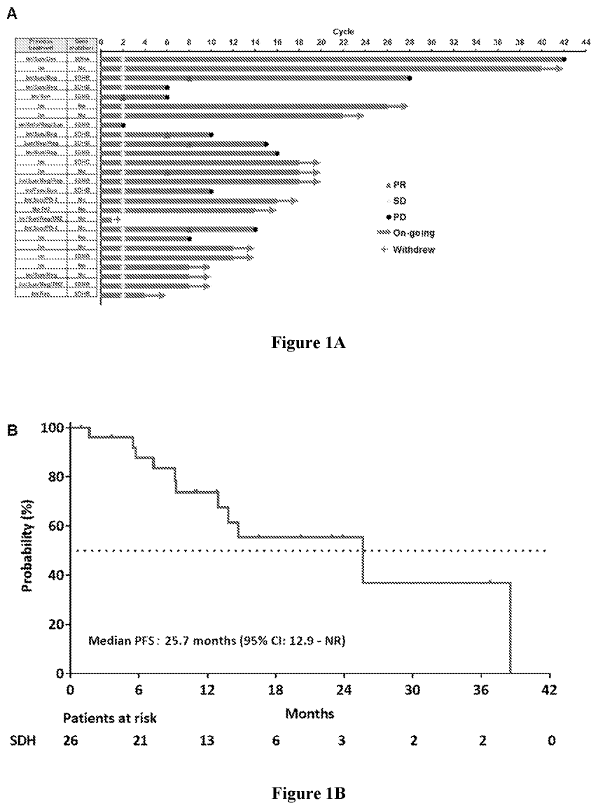

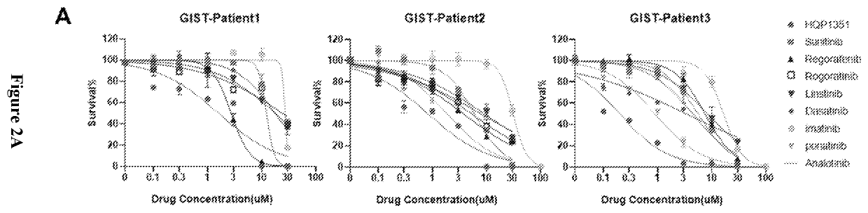

At a median follow-up of 17.0 (range, 4.1-57.5) months, the investigators confirmed overall responses per RECIST v1.1 in 6 of 26 evaluable patients in the SDH-deficient GIST subgroup. No patient experienced a complete response (CR), and 6 (23.1%) patients had a partial response (PR). Clinical benefit, defined as the proportion of patients with a confirmed CR, PR, or stable disease (SD) for ≥16 weeks, was observed in 24 (92.3%) patients (FIG. 1A). Median progression-free survival was 25.7 months (range, 12.9-not reached; FIG. 1B). At the time of data cut-off, all patients in the SDH-deficient GIST cohort survived, so the overall survival rate was 100%.

### Pharmacokinetic Analyses

Pharmacokinetics of olverembatinib were evaluated in patients with GIST after oral doses of olverembatinib (20 to 50 mg) QOD under fed conditions. Results of pharmacokinetic analysis for Day 27 are summarized in Table 6. After oral administration at doses from 20 to 50 mg, the peak concentration of olverembatinib occurred between 6 and 8 hours. The mean terminal half-life (t_{1/2}) ranged from 18.9 to 27 hours. Olverembatinib exhibited an approximately dose-proportional increase in maximum concentration (Cₘₐₓ) and area under the concentration-time curve (AUCₗₐₛₜ) over the dose range of 20 to 50 mg. There was no significant accumulation of olverembatinib after multiple-dose administration.

TABLE 6
Summary of olverembatinib pharmacokinetic parameters
at steady state in patients with GIST

| Dose | Subject | Tₘₐₓ | Cₘₐₓ | AUCₗₐₛₜ | T_{1/2} | Vz/F | Cl/F |
|---|---|---|---|---|---|---|---|
| (mg) | no. | (hr) | (ng/mL) | (h*ng/m) | (hr) | (L) | (L/hr) |
| 20 | 2 | 7 (6.00, | 9.18 ± 0.13 | 228 ± 6.4 | 22.2 ± 0.92 | 2143 ± 17.1 | 67.2 ± 2.3 |
| | | 8.00) | | | | | |
| 30 | 4 | 7 (4.00, | 12.5 ± 2.9 | 307 ± 121 | 27.0 ± 15.4 | 2649 ± 877 | 87.7 ± 56.8 |
| | | 8.00) | | | | | |
| 40 | 15 | 6 (4.00, | 21.8 ± 9.7 | 473 ± 177 | 18.9 ± 6.4 | 2126 ± 990 | 79.9 ± 35.3 |
| | | 24.0) | | | | | |
| 50 | 3 | 8 (6.00, | 26.5 ± 16.0 | 518 ± 174 | 19.5 ± 6.8 | 2349 ± 942 | 87.5 ± 39.7 |
| | | 8.00) | | | | | |

Values are median (range) for Tₘₐₓ and mean (SD) for all others.
Pharmacokinetic parameters were derived by noncompartmental analysis (NCA), AUCₗₐₛₜ, area under the curve from time zero to the time of the last measurable concentration (Tₗₐₛₜ); CL/F, apparent total oral clearance; Cₘₐₓ, maximum observed plasma drug concentration; T_{1/2}, terminal elimination half-life; Tₘₐₓ, time to reach maximum observed plasma drug concentration; Vz/F, apparent volume of distribution.

### 3. Discussion

Although the advent and use of imatinib revolutionized care for most patients with advanced GISTs, patients with SDH-deficient disease rarely respond to this agent, and other approved multikinase inhibitors have not proved efficacious. Our study, the largest global prospective clinical trial evaluating a TKI for SDH-deficient GIST (to our knowledge (Nannini, M., et al. Ther. Adv. Med. Oncol. 13, 17588359211023278 (2021))), shows that olverembatinib, a novel multikinase inhibitor, has clinical activity, with durable responses and a manageable safety profile, in patients with advanced SDH-deficient GIST, including a median PFS of 25.7 months.

Most patients with SDH-deficient GIST accrued in this study were female, younger, and had a gastric primary site of tumor origin. These clinicopathological associations of gender, SDHB expression, and tumor site are consistent with the literature concerning SDH-deficient GIST (Pasini, B., et al. Eur. J. Hum. Genet. 16, 79-88 (2008); Boikos, S. A, et al. JAMA Oncol. 7, 922-928 (2016)). Loss of SDHB protein expression occurs from biallelic inactivation of any of the SDH genes by mutation or hypermethylation of the SDHC gene promoter, which leads to SDH deficiency (Unc, M. et al. Cancers (Basel) 15, 1498 (2023); Pasini, B., et al. Eur. J. Hum. Genet. 16, 79-88 (2008); Killian, J. K., et al. Cancer Discov. 3, 648-657 (2013)). Germline mutations in SDHA occur in approximately 30% of SDH-deficient GISTs, whereas those in SDHB, SDHC, and SDHD occur in 20% to 30% of cases (Pantaleo, M. A., et al. J. Natl. Cancer Inst. 103, 983-987 (2011); Miettinen, M., & Lasota, J. Cell Biol. 53, 514-519 (2014)). The remaining 50% of SDH-deficient GISTs lack SDH mutations but show hypermethylation of the SDHC promoter (Unc, M., et al. Cancers (Basel) 15, 1498 (2023); Killian, J. K., et al. Cancer Discov. 3, 648-657 (2013)). In an observational study from the National Institutes of Health GIST Clinic that included 63 cases of SDH-mutant GIST, 34 patients had mutations in SDHA, 16 in SDHB, 12 in SDHC, and 1 in SDHD (Boikos, S. A, et al. JAMA Oncol. 7, 922-928 (2016)). In our study, germline SDHB was the most common SDH mutation (12/26 patients), with only 1 case each of germline SDHA and SDHC mutations. It is unclear why the frequency of germline SDH mutations in our study differs from previously reported values, but one potential reason is that all patients in our study were of Asian descent; additional studies are warranted to explore potential racial differences in genotype. Consistent with previous reports, we did not detect an SDH mutation in 12 of 26 (46.2%) patients with SDH-deficient GISTs, a finding that could be related to hypermethylation of the SDHC gene.

Because SDH-deficient GISTs are difficult to treat and rare, clinical experience in their management is extremely limited. Compounding this problem, most data in the literature reporting on the activity of TKIs in KIT/PDGFRA WT GISTs lack characterization of SDH deficiency. In brief, objective tumor responses in this specific GIST subtype are uncommon, and previous experience with imatinib, sunitinib, and regorafenib (type 1 or 2 TKIs) has demonstrated limited response or disease stabilization in SDH-deficient GISTs (Nannini, M., et al. Ther. Adv. Med. Oncol. 13, 17588359211023278 (2021); Boikos, S. A, et al. JAMA Oncol. 7, 922-928 (2016); Neppala, P., et al. Cancer Metastasis Rev. 38, 525-535 (2019)). Imatinib has poor efficacy in KIT/PDGFRA WT GIST (Boikos, S. A, et al. JAMA Oncol. 7, 922-928 (2016)). In a US-led phase 3 trial examining the impact of imatinib in advanced GIST, time to tumor progression was shorter for patients with WT GISTs compared with more common exon 11 mutant-positive tumors at 13 months versus 25 months, respectively (Heinrich, M. C., et al. J. Clin. Oncol. 33, 5360-5367 (2008)).

In adolescents with advanced GIST after imatinib failure, treatment with sunitinib resulted in 56% of patients experiencing stable disease (SD) for 6 months or more (Janeway, K. A, et al. Pediatr. Blood Cancer 52, 767-771 (2009)). In the aforementioned retrospective study conducted by Boikos et al (Boikos, S. A, et al. JAMA Oncol. 7, 922-928 (2016)), 7 of 38 patients with WT GIST treated with sunitinib had an objective response (18.4%), including partial responses (n=3), mixed responses (defined as progression at some sites and regression at others; n=3), and complete response (CR; n=1). In a phase 1/2 prospective trial examining sunitinib in patients aged 6 to less than 18 years with advanced GIST, no tumor responses were observed in six patients (five with SDH-deficient tumors, and one not tested), but three patients had SD, and the median PFS was only 5.8 months (Verschuur, A. C., et al. Cancer Chemother. Pharmacol. 84, 41-50 (2019)).

In a multicenter phase 2 trial of regorafenib in patients with metastatic and/or unresectable GIST after failure of standard TKI therapy, the median PFS in six patients with SDH-deficient GIST receiving regorafenib was 10 months, with all patients deriving clinical benefit for more than 16 weeks (Ben-Ami, E., et al. Ann. Oncol. 27, 1794-1799 (2016)). In a phase 2, single-arm trial of regorafenib as a single agent in the first-line setting for patients with metastatic and/or unresectable KIT/PDGFR WT GISTs, 15 patients were enrolled, and, although there were no CRs, 13% of patients experienced PRs, the disease control rate at 12 weeks was 86.7%, and the median PFS was 11 months (Martin-Broto, J., et al. Mol. Cancer 22, 127 (2023)).

Because SDH-deficient GISTs commonly have increased expression of IGF-1R, von Mehren and co-workers prospectively tested the efficacy of oral TKI IGF-1R inhibitor linsitinib in patients with WT GIST. No measurable responses were seen, but the clinical benefit rate at 9 months was 40% and the 9-month estimated PFS was 52% (von Mehren, M., et al. Clin. Cancer Res. 26, 1837-1845 (2020)). Temozolomide (TMZ), a DNA alkylating agent, has been shown to cause DNA damage and apoptosis in a preclinical study of patient-derived SDH-deficient GIST models (Yebra M., et al. Clin. Cancer Res. 28, 187-200 (2022)). Preliminary results from nine patients enrolled in a phase 2 clinical trial of TMZ in advanced SDH-deficient GISTs demonstrated a 6-month ORR of 22.2% (Burgoyne, A., et al. Proceedings of the CTOS 2022 Annual Meeting, P096, Vancouver, BC, Canada (2022)).

Considering that almost one-half of the patients in our study were heavily pretreated with more than two TKIs, it is noteworthy that patients receiving olverembatinib experienced a clinical benefit rate (CBR) of more than 90% and a mPFS of 25.7 months; this level of disease control and response durability appear to exceed all literature reports of prospective clinical trials. In addition, olverembatinib was well tolerated in this group of patients, with a frequency and severity of adverse events that were lower than in patients with CML (Jiang, Q., et al. J. Hematol. Oncol. 15, 113 (2022)). Common drug-related toxicities were chiefly grade 1 or 2 and generally manageable with standard supportive medical treatments. No dose-limiting toxicity was observed, and no patients discontinued treatment because of adverse events.

In conclusion, in this trial of prospectively enrolled patients with SDH-deficient GIST, olverembatinib was well tolerated, the CBR was more than 90%, and the estimated median PFS was significantly prolonged, indicating a potential benefit of this treatment and providing a benchmark for future studies in this rare subtype of GIST.

### Example 2. Olverembatinib in Advanced SDH-Deficient GISTs: Preclinical Results

### 1. Methods

### Cell Lines and Cell Culture

Acute lymphoblastic leukemia (Jurkat, Clone E6-1) and renal cell carcinoma (OS-RC-2) cell lines were obtained from Cell Resource Center, Shanghai Institutes for Biological Sciences, Chinese Academy of Sciences. A colon cancer cell line (RKO) was kindly gifted from Prof. Shaomeng Wang's laboratory at the University of Michigan, USA. A rat pheochromocytoma cell line (PC12) was obtained from Cobioer Biosciences Co., Ltd (Nanjing, China). The PC12 SDBH knock-down (KD) cell line (PC12 #5F7) was obtained from GENEWIZ, Inc. (Azenta Life Sciences; Suzhou, China) using an sgRNA-mediated clustered regularly interspersed short palindromic repeat (CRISPR)/Cas9 system. All cell lines were: authenticated by short-tandem repeat (STR) analysis at GENEWIZ, Inc.; tested negative for mycoplasma contamination; and, passaged in vitro for fewer than 3 months after resuscitation. All cell lines were cultured in recommended medium at 37° C. and an atmosphere of 5% CO₂ (Table 7).

TABLE 7
Cell lines and culture conditions

| Tumor type | Cell line | Culture conditions |
|---|---|---|
| Acute | Jurkat, | RPMI 1640 medium supplemented |
| lymphoblastic | Clone | with 4.5 g/L glucose, 10 mM |
| leukemia, ALL | E6-1 | HEPES, 1.0 mM sodium pyruvate, |
| | | 10% fetal bovine serum (FBS), and |
| | | 1% penicillin/streptomycin (P/S) |
| Renal cell | OS-RC-2 | RPMI 1640 medium supplemented |
| carcinoma | | with 10% FBS and 1% P/S |
| Colon cancer | RKO | Minimum essential medium |
| | | supplemented with 0.1 mM |
| | | nonessential amino acids, 1.0 mM |
| | | sodium pyruvate, 10% FBS, |
| | | and 1% P/S |
| Rat | PC12 | RPMI 1640 medium supplemented |
| pheochromocytoma | | with 5% FBS, 10% horse serum, |
| | | and 1% P/S |
| | PC12#5F7 | RPMI 1640 medium supplemented |
| | | with 5% FBS, 10% horse serum, |
| | | and 1% P/S |

HEPES, N-2-hydroxyethylpiperazine-N′-2-ethanesulfonic acid; RPMI, Roswell Park Memorial Institute.

### Human GIST Tissue Samples

Frozen GIST tissues from 15 patients were used for RNA sequencing and LC-MS, and tissues from 3 patients with SDH-deficient GIST was used for primary cell culture. All samples were collected at Sun Yat-sen University Cancer Center with informed consent obtained from each subject. The study, which involved human subjects, was approved by the Medical Ethics Committee of Sun Yat-sen University Cancer Center.

The tissues from GIST patient samples were sustained in Dulbecco's modified Eagle medium (DMEM)/F12 medium containing 1% antibiotic and washed with phosphate-buffered saline (PBS) and then cut into fragments by surgical ophthalmic scissors. The tissues were digested with 1 mg/mL collagenase IV at 37° C. for 30 minutes, followed by filtration of the suspension into single cells with a single-cell strainer centrifugation. Finally, cell suspensions were collected in a centrifuge tube, centrifuged at 500×g for 5 min, and resuspended in GIST Primary Cell Culture Medium [(DMEM)/F12 medium containing 10% FBS, 1% GlutaMAX™ (#35050061, ThermoFisher), 1% Anti-Anti (#15240062, GIBICO), 50 ng/ml SCF (#300-07-10, PPL), 125 ng/ml EGF (#PK0014-505, PPL), 20 ng/ml bFGF (#HY-P7004, MCE), 25 ng/ml Hydrocortisone (#HY-N0583, MCE), and 5 ug/ml Insulin (#HY-P0035, MCE)]. Cells were counted and seeded in 96-well plates (5,000 cells per well) and cocultured with different concentrations of olverembatinib and other TKIs for 72 hours, and then cell proliferation activity was detected using CellTiter 96® Aqueous Non-Radioactive Cell Proliferation Assay (#G7570, Promega, Madison, WI, USA). The procedures were performed as per manufacturers' instructions.

### Reagents

Olverembatinib was manufactured by Jiangsu Ascentage Pharma Group Corp. Ltd. (Taizhou, China). Imatinib, avapritinib, ponatinib, dasatinib, sunitinib, regorafenib, sorafenib, pazopanib, rogaratinib, and pemigatinib were purchased from Jiangsu Aikon Biopharmaceutical R&D Co., Ltd. Ripretinib was purchased from Selleck Chem (Houston, TX, USA). Infigratinib was purchased from MCE (MedChemExpress).

### Cell Proliferation Assay

A dose range of indicated compounds was prepared by serial dilutions and then added into 96-well plates, followed by seeding of cells at optimum densities per well. Cells were incubated with drug for 72 hours and cell growth was quantitated using the Cell Counting Kit-8 (Shanghai Liji Medical Technology Co., Ltd) or CellTiter-Glo® Luminescent Cell Viability assay kits (#G7573, Promega). For each condition, three replicates of each concentration were measured. Relative survival in the presence of drugs was normalized to untreated controls after background subtraction. Graphs were generated using Prism 9 (GraphPad Software, La Jolla, CA, USA) based on the average of three replicates.

Viability studies were carried out using the CellTiter-Glo® Luminescent assay (Promega), in which the luciferase-catalyzed luciferin/ATP reaction provides an indicator of cell number.

### Cell Death Assay

Rat pheochromocytoma cells (PC12 or PC12 SDHB KD cells PC12 #5F7) were seeded into 24-well plates and incubated overnight in complete culture medium. Cells were exposed to olverembatinib for 24 hours, then collected and resuspended in media. The cell suspension was mixed at a 1:1 ratio with Viastain™ AOPI Staining solution (acridine orange/propidium iodide, #2210170205, Nexelcom Bioscience, Lawrence, MA, USA). After staining, dead cells were counted on a Cellometer® Auto 2000 Cell Counter (Nexelcom).

### Cell Colony Formation Assay

For the colony formation assay, 1,000 cells were plated in 6-well plates per well, incubated for 7 to 14 days, then stained with 0.1% crystal violet and colony numbers counted.

### Western Blot Analysis

Harvested cells treated with dimethyl sulfoxide (DMSO) or indicated concentrations of compounds were lysed in radio-immunoprecipitation (RIPA) buffer (#P0013, Beyotime, China) containing a protease inhibitor cocktail (#20104ES08, Yeasen Biotechnology Co., Ltd., Shanghai) and a phosphatase inhibitor (#20109-A, #20109-B, Yeasen). Equal amounts of protein were separated by 4-20% sodium dodecyl sulfate polyacrylamide gel electrophoresis (SDS-PAGE) and transferred to nitrocellulose membranes. After being blocked with 3% bovine serum, membranes were incubated with primary antibodies, followed by horseradish peroxidase (HRP)-conjugated secondary antibody (#7074, anti-rabbit IgG, CST; #7076, anti-mouse IgG, CST). Finally, bound secondary antibodies were visualized using an ECL Western Blotting Detection Kit (Yeasen). The primary antibodies used included: SDHB (#AB14714, Abcam), phosphorylated FGFR1 (#52928, Cell Signaling Technology [CST]), FGFR1 (#9740, CST), FGF3 (#PA5-102906, Thermo Fisher Scientific, Waltham, MA, USA), FGF4 (#PA5-115228, Thermo Fisher Scientific), phosphorylated Src (#6943, CST), Src (#2109, CST), phosphorylated VEGFR2 (#3770, CST), VEGFR2 (#9698, CST), HIF-1α (#ab16066, Abcam), HIF-2α (#PA116510, Invitrogen), VEGFA (#ab214424, Abcam), phosphorylated AKT (#4060, CST), AKT (#4685, CST), phosphorylated ERK (#4370, CST), ERK (#4695, CST), phosphorylated IGF-1Rβ (#3021, CST), IGF-1R (#ab182408, Abcam), C-MYC (#13987, CST), NFR2 (#20733, CST), phosphorylated STAT3 (#9145, CST), STAT3 (#9139, CST), phosphorylated STAT5 (#9359, CST), STAT5 (#94205, CST), PARP-1 (#9532, CST), caspase-3 (#9665, CST), cleaved caspase-3 (#9664, CST), and β-actin (#3700, CST).

### 2. Olverembatinib had Superior Antiproliferative Activity in SDH-Deficient Tumor Cell Lines and Primary SDH-Deficient Cells.

SDH is a key respiratory mitochondrial enzyme linking the citric acid cycle and the electron transport chain. The SDH complex (also termed “mitochondrial complex II”) is composed of 4 subunits: SDHA, SDHB, SDHC, and SDHD. Biallelic inactivation of one of the four SDH subunit genes, or promoter hypermethylation and epigenetic silencing of the SDHC gene, can result in loss of function of the entire SDH-complex. Subsequently, SDHB degrades and presents as a negative immunohistochemistry for SDHB. There is no SDH-deficient GIST cell line available, and the currently available GIST cell line with mutated KIT or PDFGR also cannot be used because olverembatinib can potently inhibit mutated KIT/PDGFR, which will mask its activity through other possible pathways. Consequently, other types of SDH-deficient tumor cell lines were used to test the in vitro antitumor activity of olverembatinib. These included Jurkat clone E6-1 (SDHB mutation [mut]), OS-RC-2 (SDHA and SDHB mut), RKO (SDHA mut), and rat pheochromocytoma PC12 cells with SDHB knocked down (KD, PC12 #5F7).

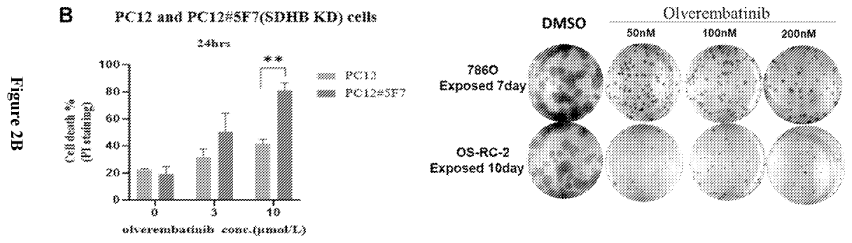

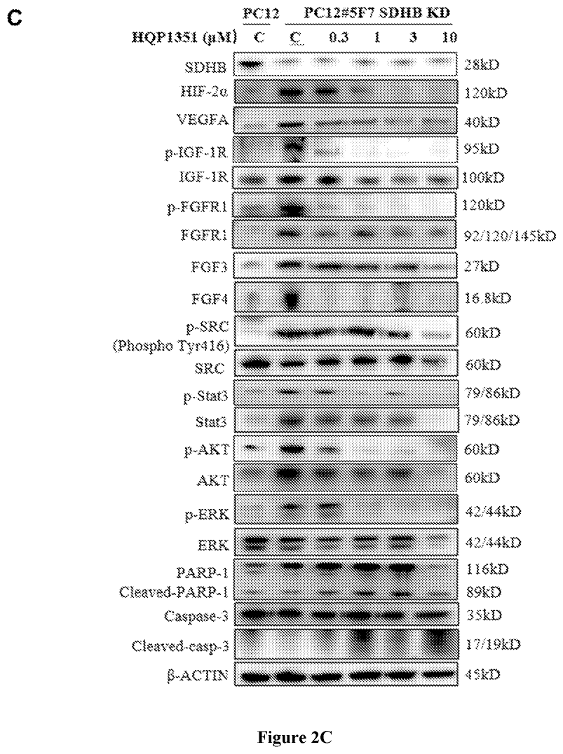

As shown in Table 8, olverembatinib exhibited superior antiproliferative activity compared to other TKIs, including imatinib, sunitinib, and regorafenib (three other KIT inhibitors approved by the Food and Drug Administration and European Medicines Agency for GIST treatment). As shown in FIG. 2A and Table 9, olverembatinib was the most potent inhibitor in all three primary cell lines derived from patients with SDH-deficient GIST.

TABLE 8
Antiproliferation activity of TKIs in SDH-deficient cell lines and primary SDH-deficient cells

| IC₅₀ μM, mean ± SD | | | |
|---|---|---|---|
| Cell lines | | | |
| | | | Rat |
| ALL | RCC | Colon cancer | pheochromocytoma |
| Jurkat, clone E6-1 | OS-RC-2 | RKO | PC12#5F7 |
| SDH status* | | | |
| | SDHA: | | |
| | missense_variant, | | |
| | p.G184R; | | |
| SDHB: | SDHB: stop | SDHA: | SDHB exon 2 + 3 |
| missense_variant, | gained, | missense_variant, | KD (heterozygote) |
| p.A15T | p.W218Ter | p.P279S | by CRISPR-Cas9 |

Compound

| | | | | |
|---|---|---|---|---|
| Olverembatinib | 0.149 ± 0.04 | 0.135 ± 0.083 | 0.129 ± 0.136 | 5.132 ± 1.950 |
| Imatinib | >10 | >10 | >10 | >10 |
| Ripretinib | 1.559 ± 0.294 | 8.781 ± 1.725 | 1.247 ± 0.933 | >10 |
| Avapritinib | >8.071 ± 2.728 | >10 ± 0 | >10 ± 0 | >10 |
| Ponatinib | 2.136 ± 2.107 | 0.809 ± 0.443 | 0.274 ± 0.241 | 8.537 ± 3.553 |
| Dasatinib | 2.025 ± 0.644 | 0.029 ± 0.003 | 0.382 ± 0.320 | >10 |
| Sunitinib | 4.289 ± 0.280 | >5.099 ± 0.207 | 3.789 ± 0.385 | >10 |
| Regorafenib | >10 | 5.110 ± 0.588 | 2.145 ± 0.812 | >10 |
| Sorafenib | >10 | 3.581 ± 0.255 | 3.875 ± 0.170 | >10 |
| Pazopanib | >10 | >10 | >10 | >10 |
| Rogaratinib | >10 | >10 | >10 | >10 |
| Infigratinib | 4.581 ± 0.342 | 3.166 ± 0.791 | 2.374 ± 2.311 | >10 |
| Pemigatinib | 4.642 ± 1.842 | >10 | >10 | >10 |
| Crenolanib | 2.814 ± 0.92 | 3.575 ± 0.771 | 3.189 ± 0.226 | >10 |
| Masitinib | 7.637 ± 3.103 | 8.581 ± 1.333 | 6.919 ± 0.837 | >10 |
| Nilotinib | 8.763 ± 0.934 | >10 | 1.454 ± 1.060 | >10 |
| Anlotinib | 1.968 ± 0.350 | 2.372 ± 0.007 | 1.464 ± 0.063 | 5.787 ± 0.452 |
| Sulfatinib | >10 | 5.806 ± 0.483 | 5.913 ± 0.739 | >10 |
| Erdafitinib | 5.425 ± 0.525 | >9.473 ± 0.746 | 6.277 ± 0.431 | >10 |
| Fisogatinib | >10 | >10 | >10 | >10 |

*From Crownbio database. ALL, acute lymphoblastic leukemia; CRISPR, clustered, regularly interspersed short palindromic repeats; IC₅₀, half-maximal inhibitory concentration; RCC, renal cell carcinoma.

TABLE 9
Antiproliferation activity of TKIs in SDH-deficient primary cells

| | GIST | GIST | GIST |
|---|---|---|---|
| | patient | patient | patient |
| Compound/IC₅₀ (μM) | 1 | 2 | 3 |
| Olverembatinib | 1.586 | 0.8365 | 0.1994 |
| Sunitinib | 12.24 | 5.976 | 5.584 |
| Regorafenib | 2.726 | 3.761 | 7.476 |
| Rogaratinib | 23.99 | 5.331 | — |
| Linsitinib | 17.61 | 7.919 | 8.513 |
| Dasatinib | 17.5 | 9.181 | 3.375 |
| Imatinib | ~26.96 | 31.09 | 16.46 |
| Ponatinib | — | 1.893 | 0.8881 |
| Anlotinib | — | — | 4.479 |

3. Tumor Cells with SDHB Deficiency or Knock-Down (KD) are More Sensitive to Olverembatinib Treatment than SDHB-Competent Cells.

To examine the activity of olverembatinib in SDH-deficient cells, we knocked down the SDHB gene by CRISPR-Cas9 in rat pheochromocytoma PC12 and generated clone 5F7 cell line (PC12 #5F7) with significantly suppressed SDHB expression. Olverembatinib demonstrated dose-dependently more potent activity in PC12 #5F7 than in parental PC12 cells (FIG. 2B, left). When comparing the activity of olverembatinib in natural SDHB-competent and deficient tumor cell lines, we found that the clone formation of OS-RC-2 (SDHB-deficient renal carcinoma) was more significantly inhibited by olverembatinib than in the 785-O (SDHB-competent renal adenocarcinoma) cell line (FIG. 2B, right panel).

### 4. Mechanisms of Action of Olverembatinib in SDH-Deficient Neoplasms.

Western blot analysis revealed that, compared to parental PC12 cells, knock-down of SDHB increased hypoxia inducible factors (HIF-1α, HIF-2α), vascular endothelial growth factor-A (VEGFA), fibroblast growth factor receptor-1 (FGFR1), insulin-like growth factor-1 receptor (IGF-1R) and phosphorylated FGFR1, and IGF-1R, all of which were reported to be involved in in vitro tumorigenesis because of deficiency of SDH (FIG. 2C). In addition, upregulation of signal transducer and activators of transcription 3 (STAT3), phosphorylated STAT3 and Src were also observed after SDHB knock-down, which has not been reported previously. Olverembatinib treatment dose-dependently inhibited these proteins as well as their downstream proteins AKT (protein kinase B) and extracellular signal-regulated kinase 1/2 (ERK1/2). These signaling pathways are involved in hypoxia, angiogenesis, proliferation, and survival. Increased cleavage of caspase-3 and poly(ADP-ribose) polymerase 1 were also observed in tumor cell lines treated with olverembatinib, suggesting induction of apoptosis.

### 5. Discussion

Although the precise oncogenic mechanisms of SDH-deficient in cancers have not been fully elucidated, research efforts to unravel the complex biological events contributing to the tumorigenesis of SDH-deficient GISTs are ongoing. The most reported mechanisms of SDH-deficient cancers involve succinate-induced signaling pathways and upregulated FGFR pathway. SDH-deficient led to accumulation of succinate, a well-established oncometabolite (Eijkelenkamp, K., et al. Clin. Genet. 97, 39-53 (2020); Kuo, C.-C., et al. J. Biomed. Sci. 29, 93 (2022); Selak, M. A. et al. Cancer Cell. 7, 77-85 (2005)), which induces the pseudohypoxia-associated proteins HIF-1α/2a, IGF-IR and angiogenic proteins (e.g., VEGF), in SDH-deficient GIST cell lines (Flavahan, W. A., et al. Nature. 575, 229-233 (2019)), paragangliomas (Gimenez-Roqueplo, A. P., et al. Am. J. Hum. Genet. 69, 1186-1197 (2001)), and pheochromocytomas (Gimenez-Roqueplo, A. P., et al. J. Clin. Endocrinol. Metab. 87, 4771-4774 (2002)). FGFR pathway signals, including receptor tyrosine kinase FGFR1 and two established oncoproteins FGF3 and FGF4, are remarkably highly expressed in SDH-deficient GISTs (Flavahan, W. A., et al. Nature. 575, 229-233 (2019)).

Upregulation of the above signal pathway proteins, which in turn govern cellular angiogenesis, proliferation, survival, and apoptosis, was also observed after SDHB knock-down in PC12 cells in our study, and these proteins, as well as their downstream signals AKT, ERK, and STAT3, could be suppressed by olverembatinib in a dose-dependent manner; these effects partially explain the MOA of olverembatinib in SDH-deficient GIST. In addition, our study also documented inhibition of the proto-oncogene tyrosine protein kinase Src by olverembatinib. Src interplays with IGF-1R to regulate the tumorigenic pathway, and its activity is increased in GIST (Rotert, J. V., et al. J. Surg. Oncol. 109, 597-605 (2014)). Furthermore, increased cleavage of caspase-3 and poly (ADP-ribose) polymerase 1 after olverembatinib treatment suggested that it induced apoptosis. Taken together, olverembatinib exerts its antitumor effects by modulating multiple signaling pathways involved in hypoxia, angiogenesis, proliferation, survival, and apoptosis.

On the basis of in vitro findings, we postulate a MOA of olverembatinib in SDH-deficient tumor cells that involves modulation of multiple signaling pathways involved in hypoxia, angiogenesis, apoptosis, proliferation, and survival. Further studies to validate these preliminary findings and elucidate the MOA of olverembatinib are still ongoing.

### Example 3. Combination of Olverembatinib (HQP1351) with BCL-2 Inhibitor Lisaftoclax (APG-2575) Overcomes Resistance in Gastrointestinal Stromal Tumors (GISTs)

Olverembatinib (HQP1351) is a new, third-generation TKI that targets BCR-ABL1, KIT and PDGFRA and is currently in development for relapsed or refractory chronic myeloid leukemia and GIST. Lisaftoclax (APG-2575) is a selective BCL-2 inhibitor under development for hematologic malignancies. The purpose of this study was to evaluate whether combining a BCL-2 inhibitor, lisaftoclax, with olverembatinib enhances treatment effect on imatinib-resistant GISTs.

Compared to imatinib-sensitive GIST cell lines (GIST882 and GIST-T1), increased BCL-2 protein expression was observed in imatinib-resistant GIST cells, particularly in GIST430, GIST48, and GIST48B lines. Cell-based antiproliferation studies showed additive activity of olverembatinib and lisaftoclax in imatinib-resistant GIST cells. In vivo studies using imatinib-resistant GIST430 cell-derived xenograft models further revealed that coadministration of olverembatinib at 15 mg/kg (every other day for 3 weeks) with lisaftoclax at 50 mg/kg (daily for 3 weeks) resulted in enhanced antitumor effects compared to either agent alone. Tumor growth inhibition (TGI) rates for combined treatment with olverembatinib and lisaftoclax reached 76.8%, which was superior to the TGI rate of either agent alone: 57.6% and 31.2% for olverembatinib and lisaftoclax, respectively. Regarding putative mechanisms, olverembatinib treatment was shown to downregulate BCL-XL and MCL-1 protein expression, possibly via inhibition of KIT signal pathway and downstream signal transducer and activator of transcription 3 (STAT3). Lisaftoclax triggered apoptosis by disrupting the BCL-2: BIM complex. Therefore, selective inhibition of BCL-2, aided by TKI-mediated MCL-1 and BCL-XL inhibition, markedly augmented cleavage of caspase-3 and poly(ADP-ribose) polymerase 1 (PARP-1), thus triggering apoptosis and subsequently enhancing antitumor effects.

Our results demonstrate that olverembatinib and BCL-2 inhibitor lisaftoclax have additive antitumor effects in imatinib-resistant GIST. This novel dual approach may have the potential for treating patients with GISTs whose disease has progressed after treatment with TKIs.

1. A method of treating a disease, disorder or condition associated with succinate dehydrogenase- (SDH-) deficiency in a subject, comprising administering a therapeutically effective amount of a multi-targeted tyrosine kinase inhibitor (TKI) to the subject, thereby treating the disease, disorder or condition associated with SDH-deficiency in the subject.

2. A method of identifying a subject having a disease, disorder or condition associated with SDH-deficiency and selecting a treatment for the identified subject, comprising:
a) determining if a biological sample from the subject is SDH-deficient;
b) identifying the subject as having the disease, disorder or condition associated with SDH-deficiency, based on the SDH-deficiency determined in step a), and
c) recommending the subject to receive treatment of a multi-targeted TKI.

3. The method of claim 1 or 2, wherein the multi-targeted TKI comprises olverembatinib.

4. The method of any one of claims 1-3, wherein the disease, disorder or condition associated with SDH-deficiency is cancer (e.g., SDH-deficient cancer).

5. The method of claim 4, wherein the cancer is selected from the group consisting of: gastrointestinal cancer (e.g., gastrointestinal stromal tumor (GIST)), cholangiocarcinoma, lung cancer, melanoma, breast cancer, colon cancer, ovarian cancer, prostate cancer, liver cancer (e.g., hepatocellular carcinoma), bladder cancer, pancreatic cancer, renal cancer, esophageal cancer, head and neck cancer, thyroid cancer, cutaneous squamous cell carcinoma, glioblastoma. neuroblastoma, urinary bladder cancer, hysterocarcinoma, melanoma, osteosarcoma, lymphoma (e.g., mantel cell lymphoma, diffuse large B cell lymphoma), leukemia (e.g., T-cell prolymphocytic leukemia, chronic lymphocytic leukemia, acute myeloid leukemia, acute lymphocytic leukemia (ALL)), multiple myeloma, uterine cancer, colorectal cancer, lung adenocarcinoma, uterine carcinosarcoma, lung squamous cell carcinoma, cervical cancer, esophagus cancer, sarcoma, chromophobe, renal cell carcinoma, clear cell RCC, papillary RCC, uveal melanoma, testicular germ cell tumor, low grade glioma (LGG), mesothelioma, pheochromocytoma, paraganglioma, thymoma, and adenoid cystic carcinoma (ACC).

6. The method of claim 4 or 5, wherein the cancer is SDH-deficient GIST.

7. The method of claim 4 or 5, wherein the SDH-deficient cancer is selected from the group consisting of SDH-deficient ALL, renal cell carcinoma, colon cancer, pheochromocytoma, thyroid neoplasia (e.g., papillary thyroid neoplasia, multifocal papillary thyroid neoplasia, differentiated thyroid cancer, papillary (follicular variant) thyroid neoplasia, follicular thyroid neoplasia), pancreatic neuroendocrine tumor/intrapancreatic paraganglioma, breast cancer (e.g., breast carcinoma (triple negative)), neuroblastoma, follicular lymphoma, b-cell lymphoma, Hodgkin's lymphoma, composite paraganglioma/neuroblastoma, rectal adenocarcinoma, Burkitt's lymphoma, thyroid paraganglioma, adrenocortical carcinoma, adenomatoid tumor of the adrenal gland, neuroblastoma, melanoma, synovial sarcoma, lung squamous cell carcinoma, embryonal rhabdomyosarcoma, lung adenocarcinoma, prostate adenocarcinoma, endometrial carcinoma, poorly differentiated carcinoma of the stomach, urothelial bladder cancer, colon adenocarcinoma, adenocarcinoma of the sigmoid colon, testicular seminoma, and pituitary adenoma.

8. The method of any one of claims 1 and 3-7, wherein the subject has been determined as having SDH-deficiency.

9. The method of any one of claims 1 and 3-8, wherein the SDH-deficiency is determined in a biological sample from the subject.

10. The method of any one of claims 1-3 and 9, wherein the biological sample is tissue (e.g., tumor tissue), fluid (e.g., whole blood, plasma, PBMC) or any biological samples that can be used for detection of germline/somatic mutation of SDH genes.

11. The method of any one of claims 1-10, wherein the SDH-deficiency is determined by immunohistochemistry, western blot, amplification-based assay, hybridization based assay, or sequencing.

12. The method of any one of claims 1-11, wherein the SDH-deficiency is indicated by presence of one or more loss of function mutations in SDHA, SDHB, SDHC, and SDHD, or any combination thereof, or indicated by SDHC promoter methylation, that causes reduction in SDH enzyme activity, destabilization of SDH protein complex, or reduction in SDH expression.

13. The method of claim 12, wherein the one or more loss of function mutations comprise a mutation selected from the group consisting of missense substitution, deletion, insertion, translocation, and premature stop codon, or any combination thereof.

14. The method of claim 12 or 13, wherein the one or more loss of function mutations in SDHB comprises one or more mutations selected from the group consisting of p.A15T, p.W218Ter, and SDHB KO exon 2+3 (Heterozygote).

15. The method of claim 12 or 13, wherein the one or more loss of function mutations in SDHB comprises one or more mutations selected from the group consisting of c.17_42dup, c.71_72insA, c.136C>T, c.137G>A, c.274T>A, c.301delT, c.380T>G, c.423+1G>A, c.423+20T>A, c.566G>T, c.600G>T, c.643-1G>C, c.725G>A, and c.778_779insG, or any combination thereof.

16. The method of claim 12 or 13, wherein the one or more loss of function mutations in SDHA comprises one or more mutations selected from the group consisting of p.G184R and p.P279S.

17. The method of claim 12 or 13, wherein the one or more loss of function mutations in SDHA comprises: c.91C>T, c.113A>T, c.160C>T, c.511C>T, c.533C>T, c.457-3_457-1delCAG, c.562C>T, c.628C>T, c.666del, c.688delG, c.767C>T, c.800C>T, c.818C>T, c.923C>T, c.985C>T, c.1043-1055del, c.1046_1047delTG, c.1054C>T, c.1151C>G, c.1215_1216insCC, c.1255G>A, c.1334C>T, c.1352G>A, c.1360G>A, c.1361C>A, c.1367C>T, c.1468delG, c.1513delA, c.1532T>C, c.1534C>T, c.1753C>T, c.1765C>T, c.1766G>A, c.1690G>A, c.1794G>C, c.1795-1G>T, c.1798C>T, and c.1799G>A, or any combination thereof.

18. The method of claim 12 or 13, wherein the one or more loss of function mutations in SDHC comprises one or more mutations selected from the group consisting of: c.1A>G, c.6delT, c.65G>A, c.160C>A, c.221A>G, c.224G>A, c.238C>T, c.301delT, c.380A>G, c.397C>T, and c.405+1G>A, or any combination thereof.

19. The method of claim 12 or 13, wherein the one or more loss of function mutations in SDHD comprises one or more mutations selected from the group consisting of: c.34G>A, c.352delG, c.416T>C, and c.455G>A, or any combination thereof.

20. The method of any one of claims 1-19, wherein the subject (e.g., the biological sample of the subject) has further been determined as having wild-type KIT, wild-type PDGFR, or combination thereof.

21. The method of any one of claims 1-20, wherein the subject (e.g., the biological sample of the subject) has been determined as comprising one or more mutations in NF1/BRAF/RAS pathway genes, PIK3CA, and fusions affecting NTRK3, or any combination thereof.

22. The method of any one of claims 1-21, wherein the subject (e.g., the biological sample of the subject) has been determined as overexpressing VEGFR or FGF ligand.

23. The method of any one of claims 1-22, wherein the subject has been determined as not sensitive to at least one existing TKI.

24. The method of claim 23, wherein the subject has not obtained complete response (CR), partial response (PR), or stable disease (SD) within 6 months of the treatment of the at least one existing TKI; or obtaining initial response or stable disease following ≥6 months of the treatment of the at least one existing TKI and subsequent progression.

25. The method of any one of claims 1-24, wherein the subject has been determined as not sensitive to at least one existing TKIs selected from the group consisting of imatinib, ripretinib, avapritinib, crenolanib, masitinib, ponatinib, dasatinib, nilotinib, sunitinib, regorafenib, sorafenib, anlotinib, sulfatinib, pazopanib, rogaratinib, infigratinib, erdafitinib, pemigatinib, fisogatinib, and famitinib, or any combinations thereof.

26. The method of any one of claims 1-25, wherein the subject has been determined as not sensitive to at least one existing TKIs selected from the group consisting of ripretinib, dasatinib, sunitinib, regorafenib, anlotinib, and famitinib, or any combinations thereof.

27. The method of any one of claims 1-26, wherein the multi-targeted TKI is capable of inhibiting at least two tyrosine kinases selected from the group consisting of: KIT, PDGFR, SRC, VEGFR (e.g., any one of VEGFR1, VEGFR2, and VEGFR3, or any combination thereof), and FGFR (e.g., any one of FGFR1, FGFR2, FGFR3, and FGFR4, or any combination thereof), or any combination thereof.

28. The method of any one of claims 1-27, wherein the multi-targeted TKI is capable of inhibiting at least two tyrosine kinases selected from the group consisting of: KIT, PDGFR, SRC, VEGFR2, and FGFR1, or any combination thereof.

29. The method of any one of claims 1-28, wherein the multi-targeted TKI is capable of inhibiting proliferation of SDH-deficient tumor cells.

30. The method of any one of claims 1-29, wherein the multi-targeted TKI has an IC₅₀ of no more than 10 μM (e.g., no more than 5 μM, 1 μM, 500 nM, 400 nM, 300 nM, 200 nM, 150 nM, 100 nM, 50 nM, 20 nM, or 10 nM) in inhibiting proliferation of SDH-deficient tumor cells, as determined by CCK8 assay or CTG assay.

31. The method of claim 29 or 30, wherein the SDH-deficient tumor cell is Jurkat clone E6-1, OS-RC-2, RKO, and PC12 #5F7, or any combination thereof.

32. The method of any one of claims 1-31, wherein the multi-targeted TKI comprises a heterocyclic alkyne benzene compound having the following formula (I): 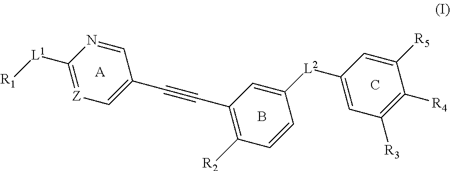
wherein Z is independently selected as CH or N; L¹ is independently selected from NH, —N═, CH; L² is independently selected as —CONH— or —NHCO—;
R₁ is independently selected from:
1) H;
2) C₁-C₆ alkyl;
3) C₃-C₆ cycloalkyl;
4) C₁-C₅ alkyl substituted by 1 or 2 hydroxy;
5) phenyl;
6) Groups which can fuse A ring through L¹ in Z atom site to form fused penta-heterocycles containing 1-3 N atoms and L¹ like 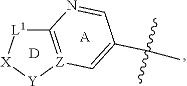
wherein, X, Y, Z are independently selected as N, CH; ring D is an aromatic heterocycle containing 1-3 N atoms;
R₂ is independently selected from
1) H;
2) Halogen;
3) C₁-C₅ alkyl;
4) C₃-C₆ cycloalkyl;
5) C₁-C₅ alkyl containing F;
R₃ is independently selected from
1) H;
2) Halogen;
3) C₁-C₄ alkyl;
4) C₃-C₆ cycloalkyl;
5) C₁-C₄ alkyl containing F;
when R₅ is H, R₄ is independently selected from
1) H;
2) (CH₂)ₙNR₆R₇;
3) (CH₂)ₙ-Het¹;
or, when R⁴ is H, R⁵ is independently selected from
1) H;
2) Het²;
n is independently selected from 0 or 1; Het¹ is defined as nonaromatic heterocycle containing 1-3 N atoms; Het² is defined as aromatic five-member heterocycle containing hetero atoms like N, O, and/or S; alkyl, cycloalkyl or NR₆R₇ may be incorporated into any C or N position in the nonaromatic heterocycle and aromatic heterocycle which can be substituted;
R₆ or R₇ is independently selected from:
1) H;
2) C₁-C₃ alkyl;
3) C₁-C₃ alkyl containing F;
4) C₃-C₆ cycloalkyl;
or R⁶ and R⁷ can further form penta-, hexa-, hepta- or octatomic ring structure through C, O, N, S atoms;
or a pharmaceutically acceptable salt or a stereoisomer or a prodrug thereof.

33. The method of any one of claims 1-32, wherein the multi-targeted TKI is a compound having the following formula (II): 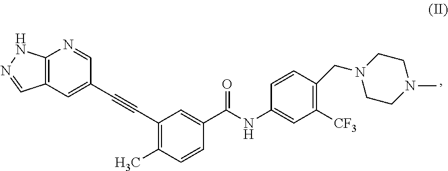 [SMILES: Cc1ccc(C(=O)Nc2ccc(CN3CCN(C)CC3)c(C(F)(F)F)c2)cc1C#Cc1cnc2[nH]ncc2c1]
or a pharmaceutically acceptable salt or a stereoisomer or a prodrug thereof.

34. The method of any one of claims 1-31, wherein the multi-targeted TKI is a compound selected from the group consisting of: olverembatinib, ripretinib, avapritinib, crenolanib, masitinib, ponatinib, dasatinib, nilotinib, sunitinib, regorafenib, sorafenib, anlotinib, sulfatinib, infigratinib, erdafitinib, pemigatinib, rogaratinib, and linsitinib, or a pharmaceutically acceptable salt or a stereoisomer or a prodrug of any of the foregoing.

35. The method of any one of claims 1-34, wherein the multi-targeted TKI is administered at a dose from about 10 mg to 200 mg, 10 mg to 150 mg, 10 mg to 100 mg, 10 mg to 50 mg, or 20 mg to 50 mg.

36. The method of any one of claims 1-35, wherein the multi-targeted TKI is administered at a dose of about 10 mg, 20 mg, 30 mg, 40 mg, or 50 mg.

37. The method of any one of claims 1-36, wherein the multi-targeted TKI is administered once a day, once every other day, once a week, once every other week, once a month, or once every other month.

38. The method of any one of claims 1-37, wherein the treatment comprises one or more 28-days treatment cycles.

39. The method of any one of claims 1-38, wherein the treatment comprises at least 1, 2, 3, 6, 14, 16 or 36 treatment cycles.

40. The method of any one of claims 1-39, wherein the multi-targeted TKI is administered by oral route or parental route (e.g., intravenous, intramuscular, subcutaneous, intramedullary, intrathecal, intraventricular, intraperitoneal, intranasal, or intraocular injection).

41. The method of any one of claims 1-40, wherein the multi-targeted TKI is orally administered once every other day in 28-days treatment cycles.

42. The method of any one of claims 1-41, wherein the method further comprises further administering a therapeutically effective amount of one or more additional therapies.

43. The method of any one of claims 1-41, wherein the multi-targeted TKI is administered in combination with one or more additional therapies that treat the disease, disorder or condition.

44. The method of claim 42 or 43, wherein the one or more additional therapies comprise a radiotherapy, a chemotherapy, a targeted cancer therapy, a therapy with a modulator of an immune checkpoint molecule, or a therapy with a BCL-2 inhibitor.

45. The method of any one of claims 1-44, wherein following the treatment of the compound having formula (II), the subject experiences partial response or stable disease.

46. The method of any one of claims 1-45, wherein following cycles of 28-days treatment cycle of the compound having formula (II), the objective response rate (complete response and partial response) is about or above 20%, and/or the clinical benefit rate (complete response, partial response and stable disease) is about or above 90%.

47. A method of treating cancer in a subject, comprising administering to the subject,
a) a therapeutically effective amount of a multi-targeted tyrosine kinase inhibitor (TKI); and
b) a therapeutically effective amount of a Bcl-2 inhibitor.

48. The method of claim 47, wherein the cancer is selected from the group consisting of: gastrointestinal cancer (e.g., gastrointestinal stromal tumor (GIST)), cholangiocarcinoma, lung cancer, melanoma, breast cancer, colon cancer, ovarian cancer, prostate cancer, liver cancer (e.g., hepatocellular carcinoma), bladder cancer, pancreatic cancer, renal cancer, esophageal cancer, head and neck cancer, thyroid cancer, cutaneous squamous cell carcinoma, glioblastoma. neuroblastoma, urinary bladder cancer, hysterocarcinoma, melanoma, osteosarcoma, lymphoma (e.g., mantel cell lymphoma, diffuse large B cell lymphoma), leukemia (e.g., T-cell prolymphocytic leukemia, chronic lymphocytic leukemia, acute myeloid leukemia, acute lymphocytic leukemia (ALL)), multiple myeloma, uterine cancer, colorectal cancer, lung adenocarcinoma, uterine carcinosarcoma, lung squamous cell carcinoma, cervical cancer, esophagus cancer, sarcoma, chromophobe, renal cell carcinoma, clear cell RCC, papillary RCC, uveal melanoma, testicular germ cell tumor, low grade glioma (LGG), mesothelioma, pheochromocytoma, paraganglioma, thymoma, and adenoid cystic carcinoma (ACC).

49. The method of claim 47 or 48, wherein the cancer is GIST.

50. The method of any one of claims 47-49, wherein the subject has been determined as comprising one or more mutations in KIT, PDGFR (e.g., PDGFRA and PDGFRB), SDH (e.g., SDHA, SDHB, SDHC and SDHD), NF1, BRAF, EGFR, HRAS, NRAS, KRAS, ZNF217, MEN1, PIK3CA, ARID1A, ARID1B, FGFR1, FGFR2, FGFR3, FGFR4, SDHAF2, FGFR1-HOOK3, FGFR1-TACC1, NTRK1 (e.g., NTRK1 fusions), NTRK2 (e.g., NTRK2 fusions), NTRK3 (e.g., NTRK3 fusions), KIT-PDGFRA, and PRKAR1B-BRAF, or any combination thereof.

51. The method of any one of claims 47-50, wherein the subject has been determined as not sensitive to at least one existing TKI.

52. The method of any one of claims 47-51, wherein the subject has not obtained complete response (CR), partial response (PR), and stable disease (SD) within 6 months of the treatment of the at least one existing TKI; or obtaining initial response or stable disease following ≥6 months of the treatment of the at least one existing TKI and subsequent progression.

53. The method of any one of claims 47-52, wherein the subject has been determined as not sensitive to at least one existing TKIs selected from the group consisting of imatinib, ripretinib, avapritinib, crenolanib, masitinib, ponatinib, dasatinib, nilotinib, sunitinib, regorafenib, sorafenib, anlotinib, sulfatinib, pazopanib, rogaratinib, infigratinib, erdafitinib, pemigatinib, fisogatinib, and famitinib, or any combination thereof.

54. The method of any one of claims 47-53, wherein the subject has been determined as not sensitive to at least one existing TKIs selected from the group consisting of ripretinib, dasatinib, sunitinib, regorafenib, anlotinib, and famitinib, or any combinations thereof.

55. The method of any one of claims 47-54, wherein the multi-targeted TKI is capable of inhibiting at least two tyrosine kinases selected from the group consisting of: KIT, PDGFR, SRC, VEGFR (e.g., any one of VEGFR1, VEGFR2, and VEGFR3, or any combination thereof), and FGFR (e.g., any one of FGFR1, FGFR2, FGFR3, and FGFR4, or any combination thereof), or any combinations thereof.

56. The method of any one of claims 47-55, wherein the multi-targeted TKI is capable of inhibiting at least two tyrosine kinases selected from the group consisting of: KIT, PDGFR, SRC, VEGFR2, and FGFR1, or any combination thereof.

57. The method of any one of claims 47-56, wherein the multi-targeted TKI comprises a heterocyclic alkyne benzene compound having the following formula (I): 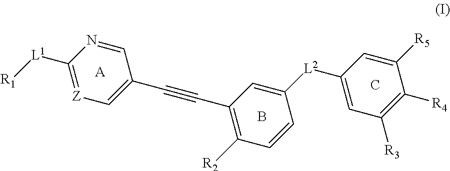
wherein Z is independently selected as CH or N; L¹ is independently selected from NH, —N═, CH; L² is independently selected as —CONH— or —NHCO—;
R₁ is independently selected from:
1) H;
2) C₁-C₆ alkyl;
3) C₃-C₆ cycloalkyl;
4) C₁-C₅ alkyl substituted by 1 or 2 hydroxy;
5) phenyl;
6) Groups which can fuse A ring through L¹ in Z atom site to form fused penta-heterocycles containing 1-3 N atoms and L¹ like 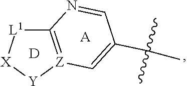
wherein, X, Y, Z are independently selected as N, CH; ring D is an aromatic heterocycle containing 1-3 N atoms;
R² is independently selected from
1) H;
2) Halogen;
3) C₁-C₅ alkyl;
4) C₃-C₆ cycloalkyl;
5) C₁-C₅ alkyl containing F;
R³ is independently selected from
1) H;
2) Halogen;
3) C₁-C₄ alkyl;
4) C₃-C₆ cycloalkyl;
5) C₁-C₄ alkyl containing F;
when R₅ is H, R₄ is independently selected from
1) H;
2) (CH₂)ₙNR₆R₇;
3) (CH₂)ₙ-Het¹;
or, when R₄ is H, R₅ is independently selected from
1) H;
2) Het²;
n is independently selected from 0 or 1; Het¹ is defined as nonaromatic heterocycle containing 1-3 N atoms; Het² is defined as aromatic five-member heterocycle containing hetero atoms like N, O, and/or S; alkyl, cycloalkyl or NR₆R₇ may be incorporated into any C or N position in the nonaromatic heterocycle and aromatic heterocycle which can be substituted;
R₆ or R₇ is independently selected from:
1) H;
2) C₁-C₃ alkyl;
3) C₁-C₃ alkyl containing F;
4) C₃-C₆ cycloalkyl;
or R₆ and R₇ can further form penta-, hexa-, hepta- or octatomic ring structure through C, O, N, S atoms;
or a pharmaceutically acceptable salt or a stereoisomer or a prodrug thereof.

58. The method of any one of claims 47-57, wherein the multi-targeted TKI is a compound having the following formula (II): 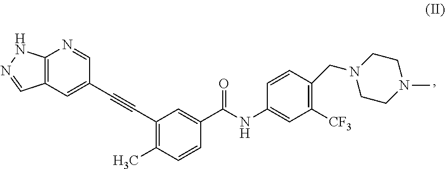 [SMILES: Cc1ccc(C(=O)Nc2ccc(CN3CCN(C)CC3)c(C(F)(F)F)c2)cc1C#Cc1cnc2[nH]ncc2c1]
or a pharmaceutically acceptable salt or a stereoisomer or a prodrug thereof.

59. The method of any one of claims 47-56, wherein the multi-targeted TKI is a compound selected from the group consisting of: olverembatinib, ripretinib, avapritinib, crenolanib, masitinib, ponatinib, dasatinib, nilotinib, sunitinib, regorafenib, sorafenib, anlotinib, sulfatinib, infigratinib, erdafitinib, pemigatinib, rogaratinib, and linsitinib, or a pharmaceutically acceptable salt or a stereoisomer or a prodrug of any of the foregoing.

60. The method of any one of claims 47-59, wherein the multi-targeted TKI is administered at a dose of about 1 mg/kg, 5 mg/kg, 10 mg/kg, 15 mg/kg, 20 mg/kg, 25 mg/kg, 50 mg/kg, 75 mg/kg, 100 mg/kg, 150 mg/kg, or 200 mg/kg.

61. The method of any one of claims 47-60, wherein the multi-targeted TKI is administered once a day, once every other day, once a week, once every other week, once a month, or once every other month.

62. The method of any one of claims 47-61, wherein the multi-targeted TKI is administered for at least 1 week, 2 weeks, 3 weeks, 4 weeks, 2 months, 3 months, or 4 months.

63. The method of any one of claims 47-62, wherein the multi-targeted TKI is administered by oral route or parental route (e.g., intravenous, intramuscular, subcutaneous, intramedullary, intrathecal, intraventricular, intraperitoneal, intranasal, or intraocular injection).

64. The method of any one of claims 47-63, wherein the multi-targeted TKI is administered once every other day for 3 weeks at a dose of about 15 mg/kg.

65. The method of any one of claims 47-64, wherein the Bcl-2 inhibitor is a compound having the following formula (III) 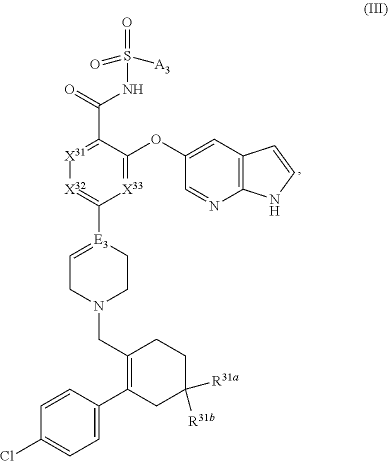
or a pharmaceutically acceptable salt or a stereoisomer or a prodrug thereof, wherein: 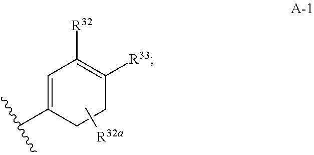
A₃ is selected from
E₃ is a nitrogen atom and [symbol] is a single bond;
X³¹, X³², and X³³ are each independently selected from the group consisting of —CR³⁸═ and —N═;
R^{31a} and R^{31b} taken together with the carbon atom to which they are attached form a 3-, 4-, or 5-membered cycloalkyl;
R³² is selected from the group consisting of —NO₂, —SO₂CH₃, and —SO₂CF₃;
R^{32a} is selected from the group consisting of hydrogen and halogen;
R³³ is selected from —N(R^{34a})(R^{34b});
R^{34a} is selected from the group consisting of optionally substituted C₁₋₆ alkyl, optionally substituted C₃₋₆ cycloalkyl, heterocyclo, heteroalkyl, (cycloalkyl)alkyl, and (heterocyclo)alkyl;
R^{34b} is selected from the group consisting of hydrogen and C₁₋₄ alkyl;
R³⁸ is selected from the group consisting of hydrogen and halogen.

66. The method of any one of claims 47-65, wherein the Bel-2 inhibitor is selected from the group consisting of: 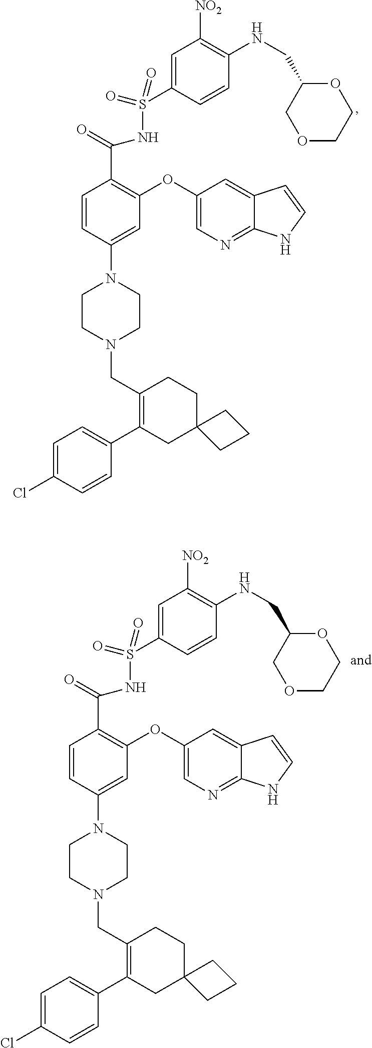 [SMILES: O=C(NS(=O)(=O)c1ccc(NC[C@H]2COCCO2)c([N+](=O)[O-])c1)c1ccc(N2CCN(CC3=C(c4ccc(Cl)cc4)CC4(CCC4)CC3)CC2)cc1Oc1cnc2[nH]ccc2c1 ; O=C(NS(=O)(=O)c1ccc(NC[C@@H]2COCCO2)c([N+](=O)[O-])c1)c1ccc(N2CCN(CC3=C(c4ccc(Cl)cc4)CC4(CCC4)CC3)CC2)cc1Oc1cnc2[nH]ccc2c1] 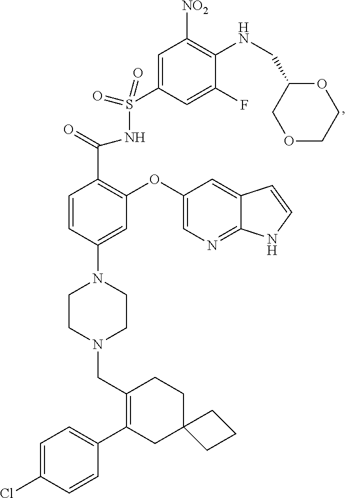 [SMILES: O=C(NS(=O)(=O)c1cc(F)c(NC[C@H]2COCCO2)c([N+](=O)[O-])c1)c1ccc(N2CCN(CC3=C(c4ccc(Cl)cc4)CC4(CCC4)CC3)CC2)cc1Oc1cnc2[nH]ccc2c1]
or a pharmaceutically acceptable salt or a stereoisomer or a prodrug thereof.

67. The method of any one of claims 47-66, wherein the Bcl-2 inhibitor is a compound having the following formula (IV) 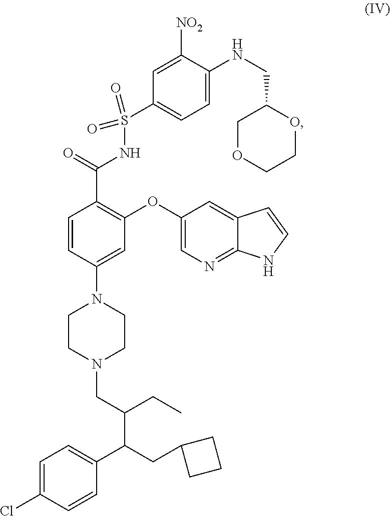 [SMILES: CCC(CN1CCN(c2ccc(C(=O)NS(=O)(=O)c3ccc(NC[C@H]4COCCO4)c([N+](=O)[O-])c3)c(Oc3cnc4[nH]ccc4c3)c2)CC1)C(CC1CCC1)c1ccc(Cl)cc1]
or a pharmaceutically acceptable salt or a stereoisomer or a prodrug thereof.

68. The method of any one of claims 47-67, wherein the Bel-2 inhibitor is administered at a dose of about 1 mg/kg, 5 mg/kg, 10 mg/kg, 15 mg/kg, 20 mg/kg, 25 mg/kg, 50 mg/kg, 75 mg/kg, 100 mg/kg, 150 mg/kg, or 200 mg/kg.

69. The method of any one of claims 47-68, wherein the Bcl-2 inhibitor is administered twice a day, once a day, once every other day, once a week, once every other week, once a month, or once every other month.

70. The method of any one of claims 47-69, wherein the Bcl-2 inhibitor is administered for at least 1 week, 2 weeks, 3 weeks, 4 weeks, 2 months, 3 months or 4 months.

71. The method of any one of claims 47-70, wherein the Bcl-2 inhibitor is administered by oral route or parental route (e.g., intravenous, intramuscular, subcutaneous, intramedullary, intrathecal, intraventricular, intraperitoneal, intranasal, or intraocular injection).

72. The method of any one of claims 47-71, wherein the Bel-2 inhibitor is administered once a day for 3 weeks at a dose of about 50 mg/kg.

73. The method of any one of claims 47-72, wherein the multi-targeted TKI and the Bcl-2 inhibitor are administered concurrently.

74. The method of any one of claims 47-72, wherein the multi-targeted TKI and the Bcl-2 inhibitor are administered sequentially.

75. The method of any one of claims 47-74, wherein the multi-targeted TKI is administered once every other day for 3 weeks at a dose of about 15 mg/kg and the Bcl-2 inhibitor is administered once a day for 3 weeks at a dose of about 50 mg/kg.

76. The method of any one of claims 47-75, wherein the multi-targeted TKI in combination with the Bel-2 inhibitor treat the cancer in the subject in an additive manner or a synergistic manner.

77. The method of any one of claims 47-76, wherein following the treatment of the compound having formula (II) in combination with the compound having formula (IV), the subject has tumor growth inhibition (TGI) rates of at least 75%.

78. The method of any one of claims 47-75, wherein the multi-targeted TKI in combination with the Bcl-2 inhibitor are capable of inhibiting proliferation of imatinib-resistant GIST cells.

79. The method of any one of claims 47-75, wherein the multi-targeted TKI in combination with the Bcl-2 inhibitor are capable of inhibiting proliferation of imatinib-resistant GIST cells in an additive manner or a synergistic manner.

80. The method of claim 78 or 79, wherein the imatinib-resistant GIST cell is GIST430, GIST48, and GIST48B, or any combination thereof.

81. A method of treating a disease, disorder or condition associated with upregulated hypoxia inducible factors (HIF-1α, HIF-2α), vascular endothelial growth factor-A (VEGFA), fibroblast growth factor receptor-1 (FGFR1), phosphorylated FGFR1, insulin-like growth factor-1 receptor (IGF-1R), phosphorylated IGF-1R, STAT3, phosphorylated STAT3 and/or phosphorylated Src in a subject, comprising administering a therapeutically effective amount of a multi-targeted tyrosine kinase inhibitor (TKI) to the subject, thereby treating the disease, disorder or condition associated with lipid metabolism in the subject.

82. The method of claim 81, wherein the disease, disorder or condition can be cancer.

83. The method of claim 82, wherein the cancer is selected from the group consisting of: gastrointestinal cancer (e.g., gastrointestinal stromal tumor (GIST)), cholangiocarcinoma, lung cancer, melanoma, breast cancer, colon cancer, ovarian cancer, prostate cancer, liver cancer (e.g. hepatocellular carcinoma), bladder cancer, pancreatic cancer, renal cancer, esophageal cancer, head and neck cancer, thyroid cancer, cutaneous squamous cell carcinoma, glioblastoma. neuroblastoma, urinary bladder cancer, hysterocarcinoma, melanoma, osteosarcoma, lymphoma (e.g., mantel cell lymphoma, diffuse large B cell lymphoma), leukemia (e.g., T-cell prolymphocytic leukemia, chronic lymphocytic leukemia, acute myeloid leukemia, acute lymphocytic leukemia (ALL)), multiple myeloma, uterine cancer, colorectal cancer, lung adenocarcinoma, uterine carcinosarcoma, lung squamous cell carcinoma, cervical cancer, esophagus cancer, sarcoma, chromophobe, renal cell carcinoma, clear cell RCC, papillary RCC, uveal melanoma, testicular germ cell tumor, low grade glioma (LGG), mesothelioma, pheochromocytoma, paraganglioma, thymoma, and adenoid cystic carcinoma (ACC).
